SEQUENCE LISTING (44 sequences)

SEQ ID NO: 1 - protein, 1368 aa, Streptococcus pyogenes [sequence omitted]

SEQ ID NO: 2 - protein, 1307 aa, Acidaminococcus sp.
MTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRN

SEQ ID NO: 3 - protein, 276 aa, Homo sapiens
SIEKEGILPNSFYEASIILIPKPGRDTTKKENFRPISLMNIDAKILNKILANRIQQHIKKLIHHDQVGFIPGMQGWFNIRKSINVIQHINRAKDKNHMIISIDAEKAFDKIQQPFMLKTLNKLGIDGTYFKIIRAIYDKPTANIILNGQKLEAFPLKTGTRQGCPLSPLLFNIVLEVLARAIRQEKEIKGIQLGKEEVKLSLFADDMIVYLENPIVSAQNLLKLISNFSKVSGYKINVQKSQAFLYTNNRQTESQIMGELPFTIASKRIKYLGIQL

SEQ ID NO: 4 - protein, 144 aa, synthetic construct
  REGION 1..144: note = Description of Artificial Sequence: Synthetic polypeptide
MKLLSSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQL

SEQ ID NO: 5 - protein, 25 aa, unidentified
  REGION 1..25: note = Description of Unknown: EGL13 NLS sequence
MSRRRKANPTKLSENAKKLAKEVEN

SEQ ID NO: 6 - protein, 10 aa, unidentified
  REGION 1..10: note = Description of Unknown: C-myc NLS sequence
VPAAKRVKLD

SEQ ID NO: 7 - protein, 2615 aa, synthetic construct
  REGION 1..2615: note = Description of Artificial Sequence: Synthetic polypeptide
MSRRRKANPTKLSENAKKLAKEVENMKLLSSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVAIPSTPPTPSPAIAMDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDGGGGGGGGGGKNLTQSRSTTWKLNNLLLNDYWVHNEMKAEIKMFFETNENKDTTYQNLWDAFKAVCRGKFIALNAYKRKQERSKIDTLTSQLKELEKQEQTHSKASRRQEITKIRAELKEIETQKTLQKINESRSWFFERINKIDRPLARLIKKKREKNQIDTIKNDKGDITTDPTEIQTTIREYYKHLYANKLENLEEMDTFLDTYTLPRLNQEEVESLNRPITGSEIVAIINSLPTKKSPGPDGFTAEFYQRYMEELVPFLLKLFQSIEKEGILPNSFYEASIILIPKPGRDTTKKENFRPISLMNIDAKILNKILANRIQQHIKKLIHHDQVGFIPGMQGWFNIRKSINVIQHINRAKDKNHMIISIDAEKAFDKIQQPFMLKTLNKLGIDGTYFKIIRAIYDKPTANIILNGQKLEAFPLKTGTRQGCPLSPLLFNIVLEVLARAIRQEKEIKGIQLGKEEVKLSLFADDMIVYLENPIVSAQNLLKLISNFSKVSGYKINVQKSQAFLYTNNRQTESQIMGELPFTIASKRIKYLGIQLTRDVKDLFKENYKPLLKEIKEETNKWKNIPCSWVGRINIVKMAILPKVIYRFNAIPIKLPMTFFTELEKTTLKFIWNQKRARIAKSILSQKNKAGGITLPDFKLYYKATVTKTAWYWYQNRDIDQWNRTEPSEIMPHIYNYLIFDKPEKNKQWGKDSLFNKWCWENWLAICRKLKLDPFLTPYTKINSRWIKDLNVKPKTIKTLEENLGITIQDIGVGKDFMSKTPKAMATKDKIDKWDLIKLKSFCTAKETTIRVNRQPTTWEKIFATYSSDKGLISRIYNELKQIYKKKTNNPIKKWAKDMNRHFSKEDIYAAKKHMKKCSSSLAIREMQIKTTMRYHLTPVRMAIIKKSGNNRCWRGCGEIGTLLHCWWDCKLVQPLWKSVWRFLRDLELEIPFDPAIPLLGIYPNEYKSCCYKDTCTRMFIAALFTIAKTWNQPKCPTMIDWIKKMWHIYTMEYYAAIKNDEFISFVGTWMKLETIILSKLSQEQKTKHRIFSLIGGNVPAAKRVKLDHHHHHH

SEQ ID NO: 8 - protein, 2554 aa, synthetic construct
  REGION 1..2554: note = Description of Artificial Sequence: Synthetic polypeptide
MSRRRKANPTKLSENAKKLAKEVENMKLLSSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVAIPSTPPTPSPAIAMTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRNGGGGGGGGGGKNLTQSRSTTWKLNNLLLNDYWVHNEMKAEIKMFFETNENKDTTYQNLWDAFKAVCRGKFIALNAYKRKQERSKIDTLTSQLKELEKQEQTHSKASRRQEITKIRAELKEIETQKTLQKINESRSWFFERINKIDRPLARLIKKKREKNQIDTIKNDKGDITTDPTEIQTTIREYYKHLYANKLENLEEMDTFLDTYTLPRLNQEEVESLNRPITGSEIVAIINSLPTKKSPGPDGFTAEFYQRYMEELVPFLLKLFQSIEKEGILPNSFYEASIILIPKPGRDTTKKENFRPISLMNIDAKILNKILANRIQQHIKKLIHHDQVGFIPGMQGWFNIRKSINVIQHINRAKDKNHMIISIDAEKAFDKIQQPFMLKTLNKLGIDGTYFKIIRAIYDKPTANIILNGQKLEAFPLKTGTRQGCPLSPLLFNIVLEVLARAIRQEKEIKGIQLGKEEVKLSLFADDMIVYLENPIVSAQNLLKLISNFSKVSGYKINVQKSQAFLYTNNRQTESQIMGELPFTIASKRIKYLGIQLTRDVKDLFKENYKPLLKEIKEETNKWKNIPCSWVGRINIVKMAILPKVIYRFNAIPIKLPMTFFTELEKTTLKFIWNQKRARIAKSILSQKNKAGGITLPDFKLYYKATVTKTAWYWYQNRDIDQWNRTEPSEIMPHIYNYLIFDKPEKNKQWGKDSLFNKWCWENWLAICRKLKLDPFLTPYTKINSRWIKDLNVKPKTIKTLEENLGITIQDIGVGKDFMSKTPKAMATKDKIDKWDLIKLKSFCTAKETTIRVNRQPTTWEKIFATYSSDKGLISRIYNELKQIYKKKTNNPIKKWAKDMNRHFSKEDIYAAKKHMKKCSSSLAIREMQIKTTMRYHLTPVRMAIIKKSGNNRCWRGCGEIGTLLHCWWDCKLVQPLWKSVWRFLRDLELEIPFDPAIPLLGIYPNEYKSCCYKDTCTRMFIAALFTIAKTWNQPKCPTMIDWIKKMWHIYTMEYYAAIKNDEFISFVGTWMKLETIILSKLSQEQKTKHRIFSLIGGNVPAAKRVKLDHHHHHH

SEQ ID NO: 9 - protein, 357 aa, Mus musculus
MAKGKRKNPTNRNQDHSPSSERSTPTPPSPGHPNTTENLDPDLKTFLMMMIEDIKKDFHKSLKDLQESTAKELQALKEKQENTAKQVMEMNKTILELKGEVDTIKKTQSEATLEIETLGKRSGTIDASISNRIQEMEERISGAEDSIENIDTTVKENTKCKRILTQNIQVIQDTMRRPNLRIIGIDENEDFQLKGPANIFNKIIEENFPNIKKEMPMIIQEAYRTPNRLDQKRNSSRHIIIRTTNALNKDRILKAVREKGQVTYKGRPIRITPDFSPETMKARRAWTDVIQTLREHKCQPRLLYPAKLSITIDGETKVFHDKTKFTQYLSTNPALQRIITEKKQYKDGNHALEQPRK

SEQ ID NO: 10 - protein, 101 aa, synthetic construct
  REGION 1..101: note = Description of Artificial Sequence: Synthetic polypeptide
LIKKKREKNQIDTIKNDKGDITTDPTEIQTTIREYYKHLYANKLENLEEMDTFLDTYTLPRLNQEEVESLNRPITGSEIVAIINSLPTKKSPGPDGFTAEF

SEQ ID NO: 11 - protein, 108 aa, synthetic construct
  REGION 1..108: note = Description of Artificial Sequence: Synthetic polypeptide
KNLTQSRSTTWKLNNLLLNDYWVHNEMKAEIKMFFETNENKDTTYQNLWDAFKAVCRGKFIALNAYKRKQERSKIDTLTSQLKELEKQEQTHSKASRRQEITKIRAEL

SEQ ID NO: 12 - DNA, 7845 bp, synthetic construct
  misc_feature 1..7845: note = Description of Artificial Sequence: Synthetic polynucleotide
  source 1..7845: mol_type = other DNA
atgagccgccgccgcaaagcgaacccgaccaaactgagcgaaaacgcgaaaaaactggcgaaagaagtggaaaacatgaagctactgtcttctatcgaacaagcatgcgatatttgccgacttaaaaagctcaagtgctccaaagaaaaaccgaagtgcgccaagtgtctgaagaacaactgggagtgtcgctactctcccaaaaccaaaaggtctccgctgactagggcacatctgacagaagtggaatcaaggctagaaagactggaacagctatttctactgatttttcctcgagaagaccttgacatgattttgaaaatggattctttacaggatataaaagcattgttaacaggattatttgtacaagataatgtgaataaagatgccgtcacagatagattggcttcagtggagactgatatgcctctaacattgagacagcatagaataagtgcgacatcatcatcggaagagagtagtaacaaaggtcaaagacagttgactgtagcgattccgtcgacaccacctactccctctccagcgatcgcaatggataaaaaatacagcattggtctggacattggcacgaatagcgttggttgggcagtgattaccgatgaatacaaagtcccgtcgaaaaaattcaaagtgctgggtaacaccgatcgccatagcattaagaaaaacctgatcggtgcgctgctgtttgattctggcgaaaccgcggaagcaacgcgtctgaaacgtaccgcacgtcgccgttacacgcgccgtaaaaatcgtatttgctatctgcaggaaatctttagcaacgaaatggcgaaagtcgatgactcatttttccaccgcctggaagaatcgtttctggtggaagaagataaaaaacatgaacgtcacccgattttcggcaatatcgttgatgaagtcgcgtaccatgaaaaatatccgacgatttaccacctgcgtaaaaaactggtggattctaccgacaaagccgatctgcgcctgatttatctggcactggctcatatgatcaaatttcgtggtcacttcctgattgaaggcgacctgaacccggataatagtgacgtcgataaactgtttattcagctggtgcaaacctataatcagctgttcgaagaaaacccgatcaatgcaagtggtgttgatgcgaaagccattctgtccgctcgcctgagtaaatcccgccgtctggaaaacctgattgcacagctgccgggtgaaaagaaaaacggtctgtttggcaatctgatcgctctgtcactgggcctgacgccgaactttaaatcgaatttcgacctggcagaagatgctaaactgcagctgagcaaagatacctacgatgacgatctggacaacctgctggcgcaaattggcgaccagtatgccgacctgtttctggcggccaaaaatctgtcagatgccattctgctgtcggacatcctgcgcgtgaacaccgaaatcacgaaagcgccgctgtcagcctcgatgattaaacgctacgatgaacatcaccaggacctgaccctgctgaaagcactggttcgtcagcaactgccggaaaaatacaaagaaattttctttgaccaaagtaaaaatggttatgcaggctacatcgatggcggtgcttcccaggaagaattctacaaattcatcaaaccgatcctggaaaaaatggatggtacggaagaactgctggtgaaactgaatcgtgaagatctgctgcgtaaacaacgcacctttgacaacggtagcattccgcatcagatccacctgggcgaactgcatgcgattctgcgccgtcaggaagatttttatccgttcctgaaagacaaccgtgaaaaaatcgaaaaaatcctgacgtttcgcatcccgtattacgttggtccgctggcacgtggtaatagccgcttcgcatggatgacccgcaaatctgaagaaaccattacgccgtggaactttgaagaagtggttgataaaggcgcaagcgctcagtcttttatcgaacgtatgaccaatttcgataaaaacctgccgaatgaaaaagtgctgccgaaacattctctgctgtatgaatactttaccgtttacaacgaactgacgaaagtgaaatatgttaccgagggtatgcgcaaaccggcgtttctgagtggcgaacagaaaaaagccattgtggatctgctgttcaaaaccaatcgtaaagttacggtcaaacagctgaaagaagattacttcaagaaaattgaatgtttcgacagcgtggaaatttctggtgttgaagatcgtttcaacgcctctctgggcacctatcatgacctgctgaaaatcatcaaagacaaagattttctggataacgaagaaaacgaagacattctggaagatatcgtgctgaccctgacgctgttcgaagatcgtgaaatgattgaagaacgcctgaaaacgtacgcacacctgtttgacgataaagttatgaaacagctgaaacgccgtcgctataccggttggggccgtctgagccgcaaactgattaatggtatccgcgataaacaatcaggcaaaacgattctggatttcctgaaatcggacggctttgccaaccgtaatttcatgcagctgatccatgacgattccctgacctttaaagaagacattcagaaagcacaagtgtcaggtcaaggcgattcgctgcatgaacacattgcgaacctggccggttcaccggctatcaaaaaaggcatcctgcagaccgtgaaagtcgtggatgaactggtgaaagttatgggtcgtcacaaaccggaaaacattgttatcgaaatggcgcgcgaaaatcagaccacgcaaaaaggccagaaaaactcgcgtgaacgcatgaaacgcattgaagaaggtatcaaagaactgggcagccagattctgaaagaacatccggtcgaaaacacccagctgcaaaatgaaaaactgtacctgtattacctgcaaaatggtcgtgacatgtatgtggatcaggaactggacatcaaccgcctgtctgactatgatgtcgaccacattgtgccgcagagctttctgaaagacgattctatcgataacaaagttctgacccgtagtgataaaaaccgcggcaaaagcgacaatgtcccgtctgaagaagttgtgaagaaaatgaaaaactactggcgtcaactgctgaatgcgaaactgattacgcagcgtaaattcgataacctgaccaaagcggaacgcggcggtctgtccgaactggataaagccggttttatcaaacgtcaactggttgaaacccgccagattacgaaacatgtcgcccagatcctggattcacgcatgaacacgaaatacgacgaaaacgataaactgatccgtgaagtcaaagtgatcaccctgaaaagtaaactggtttccgatttccgtaaagactttcagttctacaaagtccgcgaaattaacaattaccatcacgcacacgatgcttatctgaatgcagtggttggtaccgctctgatcaaaaaatatccgaaactggaaagcgaatttgtgtatggcgattacaaagtctatgacgtgcgcaaaatgattgcgaaatccgaacaggaaatcggcaaagcgaccgccaaatactttttctattcaaacatcatgaactttttcaaaaccgaaattacgctggcaaatggtgaaattcgtaaacgcccgctgatcgaaaccaacggtgaaacgggcgaaattgtgtgggataaaggccgtgacttcgcgaccgttcgcaaagtcctgtcgatgccgcaagtgaatatcgtgaagaaaaccgaagtgcagacgggcggttttagtaaagaatccatcctgccgaaacgtaacagcgataaactgattgcgcgcaaaaaagattgggacccgaaaaaatacggcggttttgatagtccgacggttgcatattccgtcctggtcgtggctaaagtcgaaaaaggtaaaagtaaaaaactgaaatccgtgaaagaactgctgggcattaccatcatggaacgtagctcttttgagaaaaacccgattgacttcctggaagccaaaggttacaaagaagtgaaaaaagatctgatcatcaaactgccgaaatatagcctgttcgaactggaaaacggccgtaaacgcatgctggcatctgctggtgaactgcagaaaggcaatgaactggcactgccgagtaaatatgttaactttctgtacctggctagccattatgaaaaactgaaaggttctccggaagataacgaacagaaacaactgttcgtcgaacaacataaacactacctggatgaaatcatcgaacagatctcagaattctcgaaacgcgtgattctggcggatgccaatctggacaaagttctgagcgcgtataacaaacatcgtgataaaccgattcgcgaacaggccgaaaatattatccacctgtttaccctgacgaacctgggcgcaccggcagcttttaaatacttcgataccacgatcgaccgtaaacgctatacctcaacgaaagaagttctggatgctaccctgattcatcaatcgatcaccggtctgtatgaaacgcgtattgatctgagtcagctgggcggtgacggtggtggtggtggtggtggtggtggtggtaagaatctcactcaaagccgctcaactacatggaaactgaacaacctgctcctgaatgactactgggtacataacgaaatgaaggcagaaataaagatgttctttgaaaccaacgagaacaaagacaccacataccagaatctctgggacgcattcaaagcagtgtgtagagggaaatttatagcactaaatgcctacaagagaaagcaggaaagatccaaaattgacaccctaacatcacaattaaaagaactagaaaagcaagagcaaacacattcaaaagctagcagaaggcaagaaataactaaaatcagagcagaactgaaggaaatagagacacaaaaaacccttcaaaaaatcaatgaatccaggagctggttttttgaaaggatcaacaaaattgatagaccgctagcaagactaataaagaaaaagagagagaagaatcaaatagacacaataaaaaatgataaaggggatatcaccaccgatcccacagaaatacaaactaccatcagagaatactacaaacacctctacgcaaataaactagaaaatctagaagaaatggatacattcctcgacacatacactctcccaagactaaaccaggaagaagttgaatctctgaatagaccaataacaggctctgaaattgtggcaataatcaatagtttaccaaccaaaaagagtccaggaccagatggattcacagccgaattctaccagaggtacatggaggaactggtaccattccttctgaaactattccaatcaatagaaaaagagggaatcctccctaactcattttatgaggccagcatcattctgataccaaagccgggcagagacacaaccaaaaaagagaattttagaccaatatccttgatgaacattgatgcaaaaatcctcaataaaatactggcaaaccgaatccagcagcacatcaaaaagcttatccaccatgatcaagtgggcttcatccctgggatgcaaggctggttcaatatacgcaaatcaataaatgtaatccagcatataaacagagccaaagacaaaaaccacatgattatctcaatagatgcagaaaaagcctttgacaaaattcaacaacccttcatgctaaaaactctcaataaattaggtattgatgggacgtatttcaaaataataagagctatctatgacaaacccacagccaatatcatactgaatgggcaaaaactggaagcattccctttgaaaaccggcacaagacagggatgccctctctcaccgctcctattcaacatagtgttggaagttctggccagggcaatcaggcaggagaaggaaataaagggtattcaattaggaaaagaggaagtcaaattgtccctgtttgcagatgacatgattgtttatctagaaaaccccatcgtctcagcccaaaatctccttaagctgataagcaacttcagcaaagtctcaggatacaaaatcaatgtacaaaaatcacaagcattcttatacaccaacaacagacaaacagagagccagatcatgggtgaactcccattcacaattgcttcaaagagaataaaatacctaggaatccaacttacaagggatgtgaaggacctcttcaaggagaactacaaaccactgctcaaggaaataaaagaggagacaaacaaatggaagaacattccatgctcatgggtaggaagaatcaatatcgtgaaaatggccatactgcccaaggtaatttacagattcaatgccatccccatcaagctaccaatgactttcttcacagaattggaaaaaactactttaaagttcatatggaaccaaaaaagagcccgcattgccaagtcaatcctaagccaaaagaacaaagctggaggcatcacactacctgacttcaaactatactacaaggctacagtaaccaaaacagcatggtactggtaccaaaacagagatatagatcaatggaacagaacagagccctcagaaataatgccgcatatctacaactatctgatctttgacaaacctgagaaaaacaagcaatggggaaaggattccctatttaataaatggtgctgggaaaactggctagccatatgtagaaagctgaaactggatcccttccttacaccttatacaaaaatcaattcaagatggattaaagatttaaacgttaaacctaaaaccataaaaaccctagaagaaaacctaggcattaccattcaggacataggcgtgggcaaggacttcatgtccaaaacaccaaaagcaatggcaacaaaagacaaaattgacaaatgggatctaattaaactaaagagcttctgcacagcaaaagaaactaccatcagagtgaacaggcaacctacaacatgggagaaaatttttgcaacctactcatctgacaaagggctaatatccagaatctacaatgaactcaaacaaatttacaagaaaaaaacaaacaaccccatcaaaaagtgggcgaaggacatgaacagacacttctcaaaagaagacatttatgcagccaaaaaacacatgaagaaatgctcatcatcactggccatcagagaaatgcaaatcaaaaccactatgagatatcatctcacaccagttagaatggcaatcattaaaaagtcaggaaacaacaggtgctggagaggatgcggagaaataggaacacttttacactgttggtgggactgtaaactagttcaaccattgtggaagtcagtgtggcgattcctcagggatctagaactagaaataccatttgacccagccatcccattactgggtatatacccaaatgagtataaatcatgctgctataaagacacatgcacacgtatgtttattgcggcactattcacaatagcaaagacttggaaccaacccaaatgtccaacaatgatagactggattaagaaaatgtggcacatatacaccatggaatactatgcagccataaaaaatgatgagttcatatcctttgtagggacatggatgaaattggaaaccatcattctcagtaaactatcgcaagaacaaaaaaccaaacaccgcatattctcactcataggtgggaatgttccggcggcgaaacgcgtgaaactggatcatcaccatcaccatcac

SEQ ID NO: 13 - DNA, 7662 bp, synthetic construct
  misc_feature 1..7662: note = Description of Artificial Sequence: Synthetic polynucleotide
  source 1..7662: mol_type = other DNA
atgagccgccgccgcaaagcgaacccgaccaaactgagcgaaaacgcgaaaaaactggcgaaagaagtggaaaacatgaagctactgtcttctatcgaacaagcatgcgatatttgccgacttaaaaagctcaagtgctccaaagaaaaaccgaagtgcgccaagtgtctgaagaacaactgggagtgtcgctactctcccaaaaccaaaaggtctccgctgactagggcacatctgacagaagtggaatcaaggctagaaagactggaacagctatttctactgatttttcctcgagaagaccttgacatgattttgaaaatggattctttacaggatataaaagcattgttaacaggattatttgtacaagataatgtgaataaagatgccgtcacagatagattggcttcagtggagactgatatgcctctaacattgagacagcatagaataagtgcgacatcatcatcggaagagagtagtaacaaaggtcaaagacagttgactgtagcgattccgtcgacaccacctactccctctccagcgatcgcaatgacccaatttgaaggttttaccaatttataccaagtttcgaagacccttcgttttgaactgattccccaaggaaaaacactcaaacatatccaggagcaagggttcattgaggaggataaagctcgcaatgaccattacaaagagttaaaaccaatcattgaccgcatctataagacttatgctgatcaatgtctccaactggtacagcttgactgggagaatctatctgcagccatagactcctatcgtaaggaaaaaaccgaagaaacacgaaatgcgctgattgaggagcaagcaacatatagaaatgcgattcatgactactttataggtcggacggataatctgacagatgccataaataagcgccatgctgaaatctataaaggactttttaaagctgaacttttcaatggaaaagttttaaagcaattagggaccgtaaccacgacagaacatgaaaatgctctactccgttcgtttgacaaatttacgacctatttttccggcttttatgaaaaccgaaaaaatgtctttagcgctgaagatatcagcacggcaattccccatcgaatcgtccaggacaatttccctaaatttaaggaaaactgccatatttttacaagattgataaccgcagttccttctttgcgggagcattttgaaaatgtcaaaaaggccattggaatctttgttagtacgtctattgaagaagtcttttcctttcccttttataatcaacttctaacccaaacgcaaattgatctttataatcaacttctcggcggcatatctagggaagcaggcacagaaaaaatcaagggacttaatgaagttctcaatctggctatccaaaaaaatgatgaaacagcccatataatcgcgtccctgccgcatcgttttattcctctttttaaacaaattctttccgatcgaaatacgttatcctttattttggaagaattcaaaagcgatgaggaagtcatccaatccttctgcaaatataaaaccctcttgagaaacgaaaatgtactggagactgcagaagcccttttcaatgaattaaattccattgatttgactcatatctttatttcccataaaaagttagaaaccatctcttcagcgctttgtgaccattgggataccttgcgcaatgcactttacgaaagacggatttctgaactcactggcaaaataacaaaaagtgccaaagaaaaagttcaaaggtcattaaaacatgaggatataaatctccaagaaattatttctgctgcaggaaaagaactatcagaagcattcaaacaaaaaacaagtgaaattctttcccatgcccatgctgcacttgaccagcctcttcccacaacattaaaaaaacaggaagaaaaagaaatcctcaaatcacagctcgattcgcttttaggcctttatcatcttcttgattggtttgctgtcgatgaaagcaatgaagtcgacccagaattctcagcacggctgacaggcattaaactagaaatggaaccaagcctttcgttttataataaagcaagaaattatgcgacaaaaaagccctattcggtggaaaaatttaaattgaattttcaaatgccaacccttgcctctggttgggatgtcaataaagaaaaaaataatggagctattttattcgtaaaaaatggtctctattaccttggtatcatgcctaaacagaaggggcgctataaagccctgtcttttgagccgacagaaaaaacatcagaaggattcgataagatgtactatgactacttcccagatgccgcaaaaatgattcctaagtgttccactcagctaaaggctgtaaccgctcattttcaaactcataccacccccattcttctctcaaataatttcattgaacctcttgaaatcacaaaagaaatttatgacctgaacaatcctgaaaaggagcctaaaaagtttcaaacggcttatgcaaagaagacaggcgatcaaaaaggctatagagaagcgctttgcaaatggattgactttacgcgggattttctctctaaatatacgaaaacaacttcaatcgatttatcttcactccgcccttcttcgcaatataaagatttaggggaatattacgccgaactgaatccgcttctctatcatatctccttccaacgaattgctgaaaaggaaatcatggatgctgtagaaacgggaaaattgtatctgttccaaatctacaataaggattttgcgaagggccatcacgggaaaccaaatctccacaccctgtattggacaggtctcttcagtcctgaaaaccttgcgaaaaccagcatcaaacttaatggtcaagcagaattgttctatcgacctaaaagccgcatgaagcggatggcccatcgtcttggggaaaaaatgctgaacaaaaaactaaaggaccagaagacaccgattccagataccctctaccaagaactgtacgattatgtcaaccaccggctaagccatgatctttccgatgaagcaagggccctgcttccaaatgttatcaccaaagaagtctcccatgaaattataaaggatcggcggtttacttccgataaatttttcttccatgttcccattacactgaattatcaagcagccaatagtcccagtaaattcaaccagcgtgtcaatgcctaccttaaggagcatccggaaacgcccatcattggtatcgatcgtggagaacgcaatctaatctatattaccgtcattgacagtactgggaaaattttggagcagcgttccctgaataccatccagcaatttgactaccaaaaaaaattggacaacagggaaaaagagcgtgttgccgcccgtcaagcctggtccgtcgtcggaacgatcaaagaccttaaacaaggctacttgtcacaggtcatccatgaaattgtagacctgatgattcattaccaagctgttgtcgtccttgaaaacctcaacttcggatttaaatcaaaacggacaggcattgccgaaaaagcagtctaccaacaatttgaaaagatgctaatagataaactcaactgtttggttctcaaagattatcctgctgagaaagtgggaggcgtcttaaacccgtatcaacttacagatcagttcacgagctttgcaaaaatgggcacgcaaagcggcttccttttctatgtaccggccccttatacctcaaagattgatcccctgactggttttgtcgatccctttgtatggaagaccattaaaaatcatgaaagtcggaagcatttcctagaaggatttgatttcctgcattatgatgtcaaaacaggtgattttatcctccattttaaaatgaatcggaatctctctttccagagagggcttcctggcttcatgccagcttgggatattgttttcgaaaagaatgaaacccaatttgatgcaaaagggacgcccttcattgcaggaaaacgaattgttcctgtaatcgaaaatcatcgttttacgggtcgttacagagacctctatcccgctaatgaactcattgcccttctggaagaaaaaggcattgtctttagagacggaagtaatatattacccaaacttttagaaaatgatgattctcatgcaattgatacgatggtcgccttgattcgcagtgtactccaaatgagaaacagcaatgccgcaacgggggaagactacatcaactctcccgttagggatctgaacggggtgtgtttcgacagtcgattccaaaatccagaatggccaatggatgcggatgccaacggagcttatcatattgccttaaaagggcagcttcttctgaaccacctcaaagaaagcaaagatctgaaattacaaaacggcatcagcaaccaagattggctggcctacattcaggaactgagaaacggtggtggtggtggtggtggtggtggtggtaagaatctcactcaaagccgctcaactacatggaaactgaacaacctgctcctgaatgactactgggtacataacgaaatgaaggcagaaataaagatgttctttgaaaccaacgagaacaaagacaccacataccagaatctctgggacgcattcaaagcagtgtgtagagggaaatttatagcactaaatgcctacaagagaaagcaggaaagatccaaaattgacaccctaacatcacaattaaaagaactagaaaagcaagagcaaacacattcaaaagctagcagaaggcaagaaataactaaaatcagagcagaactgaaggaaatagagacacaaaaaacccttcaaaaaatcaatgaatccaggagctggttttttgaaaggatcaacaaaattgatagaccgctagcaagactaataaagaaaaagagagagaagaatcaaatagacacaataaaaaatgataaaggggatatcaccaccgatcccacagaaatacaaactaccatcagagaatactacaaacacctctacgcaaataaactagaaaatctagaagaaatggatacattcctcgacacatacactctcccaagactaaaccaggaagaagttgaatctctgaatagaccaataacaggctctgaaattgtggcaataatcaatagtttaccaaccaaaaagagtccaggaccagatggattcacagccgaattctaccagaggtacatggaggaactggtaccattccttctgaaactattccaatcaatagaaaaagagggaatcctccctaactcattttatgaggccagcatcattctgataccaaagccgggcagagacacaaccaaaaaagagaattttagaccaatatccttgatgaacattgatgcaaaaatcctcaataaaatactggcaaaccgaatccagcagcacatcaaaaagcttatccaccatgatcaagtgggcttcatccctgggatgcaaggctggttcaatatacgcaaatcaataaatgtaatccagcatataaacagagccaaagacaaaaaccacatgattatctcaatagatgcagaaaaagcctttgacaaaattcaacaacccttcatgctaaaaactctcaataaattaggtattgatgggacgtatttcaaaataataagagctatctatgacaaacccacagccaatatcatactgaatgggcaaaaactggaagcattccctttgaaaaccggcacaagacagggatgccctctctcaccgctcctattcaacatagtgttggaagttctggccagggcaatcaggcaggagaaggaaataaagggtattcaattaggaaaagaggaagtcaaattgtccctgtttgcagatgacatgattgtttatctagaaaaccccatcgtctcagcccaaaatctccttaagctgataagcaacttcagcaaagtctcaggatacaaaatcaatgtacaaaaatcacaagcattcttatacaccaacaacagacaaacagagagccagatcatgggtgaactcccattcacaattgcttcaaagagaataaaatacctaggaatccaacttacaagggatgtgaaggacctcttcaaggagaactacaaaccactgctcaaggaaataaaagaggagacaaacaaatggaagaacattccatgctcatgggtaggaagaatcaatatcgtgaaaatggccatactgcccaaggtaatttacagattcaatgccatccccatcaagctaccaatgactttcttcacagaattggaaaaaactactttaaagttcatatggaaccaaaaaagagcccgcattgccaagtcaatcctaagccaaaagaacaaagctggaggcatcacactacctgacttcaaactatactacaaggctacagtaaccaaaacagcatggtactggtaccaaaacagagatatagatcaatggaacagaacagagccctcagaaataatgccgcatatctacaactatctgatctttgacaaacctgagaaaaacaagcaatggggaaaggattccctatttaataaatggtgctgggaaaactggctagccatatgtagaaagctgaaactggatcccttccttacaccttatacaaaaatcaattcaagatggattaaagatttaaacgttaaacctaaaaccataaaaaccctagaagaaaacctaggcattaccattcaggacataggcgtgggcaaggacttcatgtccaaaacaccaaaagcaatggcaacaaaagacaaaattgacaaatgggatctaattaaactaaagagcttctgcacagcaaaagaaactaccatcagagtgaacaggcaacctacaacatgggagaaaatttttgcaacctactcatctgacaaagggctaatatccagaatctacaatgaactcaaacaaatttacaagaaaaaaacaaacaaccccatcaaaaagtgggcgaaggacatgaacagacacttctcaaaagaagacatttatgcagccaaaaaacacatgaagaaatgctcatcatcactggccatcagagaaatgcaaatcaaaaccactatgagatatcatctcacaccagttagaatggcaatcattaaaaagtcaggaaacaacaggtgctggagaggatgcggagaaataggaacacttttacactgttggtgggactgtaaactagttcaaccattgtggaagtcagtgtggcgattcctcagggatctagaactagaaataccatttgacccagccatcccattactgggtatatacccaaatgagtataaatcatgctgctataaagacacatgcacacgtatgtttattgcggcactattcacaatagcaaagacttggaaccaacccaaatgtccaacaatgatagactggattaagaaaatgtggcacatatacaccatggaatactatgcagccataaaaaatgatgagttcatatcctttgtagggacatggatgaaattggaaaccatcattctcagtaaactatcgcaagaacaaaaaaccaaacaccgcatattctcactcataggtgggaatgttccggcggcgaaacgcgtgaaactggatcatcaccatcaccatcac

SEQ ID NO: 14 - protein, 1036 aa, synthetic construct
  REGION 1..1036: note = Description of Artificial Sequence: Synthetic polypeptide
KNLTQSRSTTWKLNNLLLNDYWVHNEMKAEIKMFFETNENKDTTYQNLWDAFKAVCRGKFIALNAYKRKQERSKIDTLTSQLKELEKQEQTHSKASRRQEITKIRAELKEIETQKTLQKINESRSWFFERINKIDRPLARLIKKKREKNQIDTIKNDKGDITTDPTEIQTTIREYYKHLYANKLENLEEMDTFLDTYTLPRLNQEEVESLNRPITGSEIVAIINSLPTKKSPGPDGFTAEFYQRYMEELVPFLLKLFQSIEKEGILPNSFYEASIILIPKPGRDTTKKENFRPISLMNIDAKILNKILANRIQQHIKKLIHHDQVGFIPGMQGWFNIRKSINVIQHINRAKDKNHMIISIDAEKAFDKIQQPFMLKTLNKLGIDGTYFKIIRAIYDKPTANIILNGQKLEAFPLKTGTRQGCPLSPLLFNIVLEVLARAIRQEKEIKGIQLGKEEVKLSLFADDMIVYLENPIVSAQNLLKLISNFSKVSGYKINVQKSQAFLYTNNRQTESQIMGELPFTIASKRIKYLGIQLTRDVKDLFKENYKPLLKEIKEETNKWKNIPCSWVGRINIVKMAILPKVIYRFNAIPIKLPMTFFTELEKTTLKFIWNQKRARIAKSILSQKNKAGGITLPDFKLYYKATVTKTAWYWYQNRDIDQWNRTEPSEIMPHIYNYLIFDKPEKNKQWGKDSLFNKWCWENWLAICRKLKLDPFLTPYTKINSRWIKDLNVKPKTIKTLEENLGITIQDIGVGKDFMSKTPKAMATKDKIDKWDLIKLKSFCTAKETTIRVNRQPTTWEKIFATYSSDKGLISRIYNELKQIYKKKTNNPIKKWAKDMNRHFSKEDIYAAKKHMKKCSSSLAIREMQIKTTMRYHLTPVRMAIIKKSGNNRCWRGCGEIGTLLHCWWDCKLVQPLWKSVWRFLRDLELEIPFDPAIPLLGIYPNEYKSCCYKDTCTRMFIAALFTIAKTWNQPKCPTMIDWIKKMWHIYTMEYYAAIKNDEFISFVGTWMKLETIILSKLSQEQKTKHRIFSLIGGN

SEQ ID NO: 15 - protein, 16 aa, synthetic construct
  REGION 1..16: note = Description of Artificial Sequence: Synthetic peptide
TVAIPSTPPTPSPAIA

SEQ ID NO: 16 - protein, 18 aa, synthetic construct
  REGION 1..18: note = Description of Artificial Sequence: Synthetic peptide
CWRGCGEIGTLLHCWWDC

SEQ ID NO: 17 - protein, 6 aa, synthetic construct
  REGION 1..6: note = Description of Artificial Sequence: Synthetic 6xHis tag
HHHHHH

SEQ ID NO: 18 - protein, 5 aa, synthetic construct
  REGION 1..5: note = Description of Artificial Sequence: Synthetic peptide
GGGGS

SEQ ID NO: 19 - protein, 15 aa, synthetic construct
  REGION 1..15: note = Description of Artificial Sequence: Synthetic peptide
GGGGSGGGGSGGGGS

SEQ ID NO: 20 - DNA, 51 bp, synthetic construct
  misc_feature 1..51: note = Description of Artificial Sequence: Synthetic oligonucleotide
  source 1..51: mol_type = other DNA
gtggaattgtgagcggataacaatttcacacaggaaacagctatgaccatg

SEQ ID NO: 21 - DNA, 35 bp, synthetic construct
  misc_feature 1..35: note = Description of Artificial Sequence: Synthetic oligonucleotide
  source 1..35: mol_type = other DNA
aacaatttcaatttacgttgacaccacctttcgcg

SEQ ID NO: 22 - DNA, 34 bp, synthetic construct
  misc_feature 1..34: note = Description of Artificial Sequence: Synthetic oligonucleotide
  source 1..34: mol_type = other DNA
aacaatttcatttacgttgacaccacctttcgcg

SEQ ID NO: 23 - DNA, 33 bp, synthetic construct
  misc_feature 1..33: note = Description of Artificial Sequence: Synthetic oligonucleotide
  source 1..33: mol_type = other DNA
aacaatttcattacgttgacaccacctttcgcg

SEQ ID NO: 24 - DNA, 33 bp, synthetic construct
  misc_feature 1..33: note = Description of Artificial Sequence: Synthetic oligonucleotide
  source 1..33: mol_type = other DNA
aacaatttatttacgttgacaccacctttcgcg

SEQ ID NO: 25 - DNA, 35 bp, synthetic construct
  misc_feature 1..35: note = Description of Artificial Sequence: Synthetic oligonucleotide
  source 1..35: mol_type = other DNA
gatcaccaaggtagtcggcaaataacacaggaaac

SEQ ID NO: 26 - DNA, 40 bp, Escherichia coli
  source 1..40: mol_type = unassigned DNA
tttatatattcttgccacgcaacactctatctcattattt

SEQ ID NO: 27 - DNA, 40 bp, Escherichia coli
  source 1..40: mol_type = unassigned DNA
cccgctcaaacacgccattctgctcaaaacagtaaccgcc

SEQ ID NO: 28 - DNA, 40 bp, Escherichia coli
  source 1..40: mol_type = unassigned DNA
attccggtgatcgacaccggaaactctgcttcaatctcac

SEQ ID NO: 29 - DNA, 40 bp, Escherichia coli
  source 1..40: mol_type = unassigned DNA
gctgcgcacccggggaattgagcttaaaaattggccaata

SEQ ID NO: 30 - DNA, 40 bp, Escherichia coli
  source 1..40: mol_type = unassigned DNA
atattgattgtggatctcagccggaccatccagcgataac

SEQ ID NO: 31 - DNA, 40 bp, Escherichia coli
  source 1..40: mol_type = unassigned DNA
gccccaaggtccaaacggtgattaagacccactttcacat

SEQ ID NO: 32 - DNA, 40 bp, Escherichia coli
  source 1..40: mol_type = unassigned DNA
attctcgtattaccagtttcccatacaaaagtaatgcacc

SEQ ID NO: 33 - DNA, 40 bp, Escherichia coli
  source 1..40: mol_type = unassigned DNA
atttaccgatatgtgcgaaggcttaccggaaaaaagactt

SEQ ID NO: 34 - DNA, 40 bp, Escherichia coli
  source 1..40: mol_type = unassigned DNA
tcaccggtcatcgccagatggtagccattcagcccgttgt

SEQ ID NO: 35 - DNA, 40 bp, Escherichia coli
  source 1..40: mol_type = unassigned DNA
caccattgcggcagcgtcagaaatgcctgcactcattatg

SEQ ID NO: 36 - DNA, 40 bp, Escherichia coli
  source 1..40: mol_type = unassigned DNA
attccggcaagggtctgatgttcactcagccgaatcaggc

SEQ ID NO: 37 - DNA, 40 bp, Escherichia coli
  source 1..40: mol_type = unassigned DNA
aaataatgatgtgtataaagaactgaaaaagtcactcagc

SEQ ID NO: 38 - DNA, 40 bp, Escherichia coli
  source 1..40: mol_type = unassigned DNA
tatcgtgacctgcggaaatcaccggtatgccaacgggtaa

SEQ ID NO: 39 - DNA, 40 bp, Escherichia coli
  source 1..40: mol_type = unassigned DNA
acaccactttatgcacgttcgcgctcaagctgccccacgg

SEQ ID NO: 40 - DNA, 40 bp, Escherichia coli
  source 1..40: mol_type = unassigned DNA
ttgcgctgatcgtcgtcggcaagaaactcactggcgatga

SEQ ID NO: 41 - RNA, 30 nt, synthetic construct
  misc_feature 1..30: note = Description of Artificial Sequence: Synthetic oligonucleotide
  source 1..30: mol_type = other RNA
aaaaaaaaaaaaaaaaaaaaaaaaaaaaaa

SEQ ID NO: 42 - RNA, 100 nt, synthetic construct
  misc_feature 1..100: note = Description of Artificial Sequence: Synthetic polynucleotide
  source 1..100: mol_type = other RNA
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 43 - protein, 4 aa, synthetic construct
  REGION 1..4: note = Description of Artificial Sequence: Synthetic peptide
GGGG

SEQ ID NO: 44 - protein, 10 aa, synthetic construct
  REGION 1..10: note = Description of Artificial Sequence: Synthetic peptide
GGGGGGGGGG

(19) United States
(12) Patent Application Publication
(10) Pub. No.: US 2026/0234202 A1
(43) Pub. Date: Aug. 13, 2026

(54) RETROTRANSPOSON RELATED GENOME EDITING MATERIALS AND METHODS

(71) Applicant: The Regents of the University of California, Oakland, CA (US)

(72) Inventor: Thomas E. Kuhlman, Sylmar, CA (US)

(21) Appl. No.: 19/352,179

(22) Filed: Oct. 7, 2025

Related U.S. Application Data
(63) Continuation of application No. 17/073,049, filed on Oct. 16, 2020, now abandoned.
(60) Provisional application No. 62/916,041, filed on Oct. 16, 2019.

(51) Int. Cl. C07K 14/47; C12N 9/22; C12N 15/113

(52) U.S. Cl. CPC C07K 14/4702; C12N 9/22; C12N 15/113; C07K 2319/30

(57) ABSTRACT

Certain embodiments of the invention provide a recombinant polypeptide comprising a Cas nuclease amino acid sequence operably linked to an ORF2p reverse transcriptase (RT) amino acid sequence, as well as methods of using such a recombinant polypeptide for genomic editing.

### CROSS-REFERENCE TO RELATED APPLICATIONS

This application is a continuation of U.S. application Ser. No. 17/073,049, filed Oct. 16, 2020, which claims benefit of U.S. Provisional Application Ser. No. 62/916,041 filed on Oct. 16, 2019, which applications are incorporated by reference in their entirety for all purposes.

### SEQUENCE LISTING

The instant application contains a Sequence Listing which has been submitted electronically in XML file format and is hereby incorporated by reference in its entirety. Said XML copy, created on Mar. 26, 2026, is named 12111_015US2_SL.xml and is 75,723 bytes in size.

### BACKGROUND OF THE INVENTION

The goal of genome editing technologies relates to the precise integration of gene-sized DNA fragments into an organism's genome. While many technologies exist to insert gene-sized large DNA fragments (e.g., longer than 1 kb) in simple organisms, e.g. E. coli, S. cerevisiae, etc., reliable modification in more complex cells, e.g. human or other mammalian cells, remains a challenge.

In complex eukaryotic cells, site specific integration of DNA fragments is currently performed primarily through Homology Directed Repair (HDR). In HDR, a double strand break (DSB) is introduced at the desired integration site through CRISPR-Cas9 or similarly targeted endonuclease. Simultaneously, the desired linear DNA fragment to be inserted is also delivered to the cell. The DNA fragment is generally designed such that each end of the fragment bears considerable (100-1000 bp) homology to the sequence flanking the desired integration site. This homology is subsequently detected by the cell's homologous recombination repair machinery and the fragment is integrated using the homology as a guide. One limitation of HDR is that the efficiency of insertion decreases rapidly with increasing insert size. Insertions of fragments greater than ~500 bp rapidly becomes prohibitively difficult.

Thus, there is a need for new genome editing tools to provide improved efficiency in incorporating gene-sized DNA fragments.

### SUMMARY OF THE INVENTION

Certain embodiments provide a recombinant polypeptide comprising a Cas nuclease amino acid sequence operably linked to an ORF2p reverse transcriptase (RT) amino acid sequence.

Certain embodiments also provide a nucleic acid encoding a recombinant polypeptide as described herein. Certain embodiments provide an expression cassette comprising a nucleic acid described herein. Certain embodiments provide a vector comprising a nucleic acid described herein.

Certain embodiments provide a genome editing system comprising the following components: a guide RNA or a vector comprising a nucleic acid encoding the guide RNA; a payload RNA or a vector comprising a nucleic acid encoding the payload RNA; and a recombinant polypeptide as described herein or a vector comprising a nucleic acid encoding the recombinant polypeptide, wherein the vector optionally further comprises a nucleic acid encoding ORF1p.

Certain embodiments provide a kit comprising a genome editing system described herein; packaging materials; and instructions for editing a target genomic sequence in a cell by contacting the cell with the genome editing system under conditions suitable for the components of the system to enter the cell and edit the target genomic sequence.

Certain embodiments provide a genome editing complex comprising a recombinant polypeptide described herein and a guide RNA, wherein the recombinant polypeptide forms a protein-RNA complex with the guide RNA.

Certain embodiments provide a genome editing complex comprising a recombinant polypeptide described herein, a payload RNA and a target genome sequence, wherein the recombinant polypeptide forms a protein-RNA-DNA complex with the payload RNA and the target genome sequence. Certain embodiments also provide a payload RNA that is capable of forming the genome editing complex.

Certain embodiments provide a method of editing a target genomic sequence in a cell comprising contacting the cell with a genome editing system described herein under conditions suitable for the components of the system to enter the cell and edit the target genomic sequence.

Certain embodiments provide a method of treating a disease or disorder in a mammal in need thereof, comprising administering to the mammal a genome editing system described herein.

As provided herein are processes and intermediates disclosed herein that are useful for preparing components of the a genome editing system described herein.

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIGS. 1A-1F. The GENEWRITE System. (FIG. 1A) Top: organization of human retrotransposon LINE-1, encoding the two retrotransposon proteins ORF1p and ORF2p. Middle: Domain structure of ORF2p, with Endonuclease (EN) domain at the N-terminus. Bottom: Modified ORF2p to generate one embodiment of the GENEWRITE protein, with endogenous ORF2p EN domain or EN function replaced by a Cas endonuclease (e.g., Cas9 or Cas12a/Cpf1), while the ORF2p Z domain, ORF2p reverse transcriptase (RT) and ORF2p Cys domain are preserved in this example. Additional modifications (e.g., N-terminus and/or C-terminus nuclear localization signal (NLS) sequences, and a GAL4 DNA binding domain (DBD) sequence) are also indicated. (FIG. 1B) Primary components of the GENEWRITE system (a guide RNA, a payload RNA and GENEWRITE protein) and a double stranded target DNA sequence having an insertion target are shown. Specifically, the “target genome guide sequence” (red), which is present in the “Insertion Target”, includes the desired cut site and is targeted by the “Guide RNA” (red). The “target genome homology sequence” (green), which is present in the “Insertion Target”, is homologous to and can hybridize with the payload RNA 3′ homology end (green). The “Insertion Target” also has DNA sequences flanking the “target genome guide sequence” and the “target genome homology sequence”. The flanking DNA sequences are shown in black. (FIG. 1C) The “Guide RNA” in complex with the Cas nuclease portion of GENEWRITE directs cleavage at the desired cut/integration site. (FIG. 1D) The 3′ homology end of the payload RNA hybridizes with the target genome homology sequence. (FIG. 1E) The RT component of GENEWRITE protein reverse transcribes the payload sequence for incorporation into the integration site. (FIG. 1F) The cut is sealed by non-homologous end joining (NHEJ), completing integration of the payload.

FIG. 2. Verification of Site-specific Integration of aadA by GENEWRITE. Primers were used to amplify the junctions between the lac promoter and aadA resistance gene for four colonies putatively positive for aadA integration by GENEWRITE. For each colony, PCR amplification of the 5′ junction is in the first lane (300 bp amplicon expected), and amplification of the 3′ junction is in the second lane (400 bp amplicon expected). The negative control is the same PCR reactions performed on a colony of BL21-AI pUC57-kan-CRT pZA31-NHEJ that have not gone through the integration protocol and are not spectinomycin resistant.

FIG. 3. Design of Payload RNA. The average number of successful colonies resulting from the GENEWRITE procedure as a function of the length of 3′ homology between the payload RNA and insertion site. Values given are the average of three replicates, with error bars giving the standard deviation. Dot-dash: payload with 30 bp poly-A tract (SEQ ID NO: 41) and without expression of NHEJ enzymes (−NHEJ+poly(A)); Long dash: payload without 30 bp poly-A tract (SEQ ID NO: 41) and without NHEJ expression (−NHEJ); Short dash: payload with poly-A and with expression of NHEJ (+NHEJ+poly(A)); Dotted: no poly-A payload, with NHEJ and ORF1p expression (+NHEJ+ORF1); Solid line: no poly-A payload, with NHEJ expression (+NHEJ).

FIGS. 4A-4B. Strong GENEWRITE expression is lethal to E. coli without NHEJ. (FIG. 4A) Cultures of E. coli carrying pUC57-kan-GENEWRITE (GW) either with (+) or without (−) B. subtilis NHEJ genes, and with (+) or without (−) induction with 0.4% w/v L-arabinose. Cultures were grown at 37 C in a shaking water bath for 18 hours in Rich Defined Medium (RDM)+0.5% v/v glycerol as carbon source. (FIG. 4B) Growth rates (doublings per hour) of BL21-AI expressing GENEWRITE (GW), only Cas9, or only ORF2p RT EN-under indicated conditions.

FIGS. 5A-5D. Specific GENEWRITE insertion. sgRNA and payload RNAs were designed to integrate an aadA spectinomycin resistance gene into the plasmid pUC57-kan. (FIG. 5A) Number of spectinomycin resistant colonies as a function of payload RNA 3′hybridization length. Dark red: −NHEJ+poly(A); Dark cyan:−NHEJ-poly(A); Bright red: +NHEJ+poly(A); Bright cyan: +NHEJ-poly(A). Data points are the average of three replicates. (FIG. 5B) PCR verification of integration with 40 bp homology. Lanes 1, 4, 13: NEB 1 kb Plus Ladder. 300 and 400 bp bands are indicated. Lanes 5-12: amplicons resulting from four spectinomycin resistant colonies. For each pair of lanes, the leftmost lane is PCR across the 5′ junction (300 bp amplicon expected), rightmost is PCR across 3′ junction (400 bp amplicon expected). (FIG. 5C) Representative screening of 16 randomly selected colonies by colony PCR across the 5′ integration junction. Expected 300 bp amplicon size is indicated. (FIG. 5D) Top: Sequence of target site and design features (SEQ ID NO: 20). Note that guide sequence+PAM is destroyed upon successful integration. Bottom: Sequencing of eight positive clones, with mismatches highlighted in red (SEQ ID NOS 21, 25, 21, 25, 22, 25, 21, 25, 23, 25, 24, 25, 21, 25, 21 and 25, respectively, in order of appearance).

FIG. 6. Controls and GENEWRITE variants. Column (1): standard GENEWRITE with Cas9, as in FIG. 5; (2) sgRNA only, no payload delivered; (3) payload RNA only, no guide; (4) standard GENEWRITE, but with no GAL4 domain; (5) GENEWRITE with Cas9 replaced with Cas12a/Cpf1.

FIG. 7. Chromosomal integration at the nth locus. Expected band size from null result is ~150 bp, while successful integration should yield ~1500 bp (indicated by white arrow). One identified correct integrant is indicated with green arrow.

FIGS. 8A-8B. Structure and function of LINE-1. (FIG. 8A) LINE-1 codes for two genes, ORF1 and ORF2, followed by an ~100 bp poly(A) tract (SEQ ID NO: 42). (FIG. 8B) Mechanism of TPRT (i) First-strand cleavage; (ii) annealing of RNA; (iii) reverse transcription; (iv) removal of RNA; (v) second-strand cleavage; (vi) initiation of second-strand synthesis; (vii) remaining DNA synthesis.

FIGS. 9A-9B. LINE-1 integration locations in E. coli. 12 LINE-1 integration sites in E. coli identified by Illumina sequencing with 150 bp paired-end reads. (FIG. 9A) Sequences upstream and downstream of identified insertion locations. C/G immediately upstream of insertion is highlighted red, TA-rich regions downstream are highlighted green. FIG. 9A discloses SEQ ID NOS 26-40, respectively, in order of appearance. (FIG. 9B) Logo plot of 20 bp surrounding integration site. Note G/C immediately upstream of insertion site is most prominent feature.

### DETAILED DESCRIPTION

A retrotransposon is a mobile element that can “copy and paste” itself in the genome. Unlike transposons that can “cut and paste” and jump within the genome without changing copy numbers, retrotransposons are able to amplify themselves via an RNA intermediate by initially transcribing itself into RNA, which is then used as a template for reverse transcription into DNA for insertion into the genome. Retrotransposons are a ubiquitous genomic component of many eukaryotic organisms from plants to mammals. It has been estimated that approximately 40% of the human genome is made up of retrotransposons.

For example, in the human genome, retrotransposon Long Interspersed Nucleotide Element-1 (LINE-1 or L1) replicates itself through a transcribed RNA intermediate. LINE-1 is transcribed into RNA, and the two genes it encodes, ORF1 and ORF2, are translated into the proteins ORF1p and ORF2p, respectively. ORF2p has two primary domains: an endonuclease domain (EN) at the N terminal end, and a reverse transcriptase (RT) domain towards the C terminal end. ORF1p and ORF2p bind preferentially in cis to their encoding mRNA, and the resulting ribonucleoprotein particle (RNP) enters the nucleus and binds to the host DNA. The degenerate consensus recognition sequence for ORF2p EN is 5′-TTTT/A-3′ (along with variants), which is encountered frequently within AT-rich DNA. ORF2p EN cuts the DNA at the TpA bond (indicated by a slash above). The short poly-A tail (e.g., AAAA) of the LINE-1 mRNA hybridizes with the T-rich host DNA (e.g., TTTT) at the cut, and this DNA/RNA hybridization, along with the free 3′ hydroxyl resulting from the cut, serve as a primer for the ORF2p RT domain. ORF2p RT reverse transcribes the LINE-1 mRNA into DNA, inserting a new copy of LINE-1 within the host genome in a process called target primed reverse transcription (TPRT) (Moran J V, Et al. Cell 87, 917-927 (1996)). Because of the frequency of the EN target sequence in the host genome and the promiscuity of the EN domain, insertion occurs in pseudo-random locations throughout the host genome. Thus, naturally occurring retrotransposon LINE-1 machinery lacks the specificity required for targeted genome editing.

### Targeted Retrotransposon Recombinant Polypeptide

Thus, in certain embodiments of the present invention a native retrotransposon EN domain is replaced with an exogenous targeted nuclease to engineer a non-naturally occurring recombinant retrotransposon polypeptide that confers programmable target site-specificity to cut at desired genome integration sites and initiate retrotransposon protein mediated reverse transcription of a payload sequence for genomic integration.

In certain embodiments, the native retrotransposon protein EN domain sequence is fully or partially replaced with an exogenous targeted nuclease sequence. In certain embodiments, the function of the native retrotransposon EN is inactivated and/or replaced by the exogenous targeted nuclease sequence. In certain embodiments, without a functional native retrotransposon EN cutting the conventional retrotransposon consensus recognition sequence (e.g., 5′-TTTT/A-3′), the programmable targeted nuclease confers versatile target site-specificity and directs the engineered retrotransposon protein to the desired genomic target site for integration of a payload sequence.

In certain embodiments, the recombinant polypeptide comprises a targeted nuclease amino acid sequence operably linked to a retrotransposon reverse transcriptase (RT) amino acid sequence.

In certain embodiments, the present invention provides a recombinant polypeptide for engineered retrotransposon mediated integration of a payload sequence at a site-specific genomic target. For example, in certain embodiments, the recombinant polypeptide comprises a targeted nuclease amino acid sequence operably linked to a portion of the native retrotransposon protein, which lacks its functional native EN, wherein the site-specificity of the retrotransposon mediated integration is enhanced by the targeted nuclease amino acid sequence.

In certain embodiments, the present invention provides a Cas-ORF2p retrotransposon recombinant polypeptide comprising a Cas nuclease amino acid sequence described herein and an ORF2p retrotransposon reverse transcriptase (ORF2p RT) amino acid sequence as described herein.

In certain embodiments, the present invention provides a recombinant polypeptide for ORF2p mediated integration of a payload sequence at a genomic target site. In certain embodiments, the recombinant polypeptide comprises a Cas nuclease amino acid sequence operably linked to an ORF2p RT amino acid sequence, wherein the target site-specificity of the ORF2p mediated integration is enhanced by the Cas nuclease amino acid sequence.

In certain embodiments, the recombinant polypeptide does not comprise an ORF2p EN domain amino acid sequence. In certain embodiments, the recombinant polypeptide comprises a portion of the ORF2p EN domain amino acid sequence, wherein the EN domain is not functional.

In certain embodiments, the Cas nuclease is a CRISPR-Cas nuclease. In certain embodiments, the CRISPR-Cas nuclease is a CRISPR-Cas9 nuclease or a CRISPR-Cas12a nuclease. In certain embodiments, the Cas9 nuclease is derived from S. pneumoniae, S. pyogenes, S. thermophiles, F. novicida, S. aureus, N. meningitidis or C. jejuni Cas9, and may include mutations as a Cas9 variant. In some embodiments, the Cas9 nuclease is SpCas9, SaCas9, StCas9, NmeCas9, or CjCas9. In some embodiments, the Cas12a nuclease is derived from L. bacterium or Acidaminococcus sp. and may include mutations as a Cas12a variant. In some embodiments, the Cas12a nuclease is LpCpf1 or AsCpf1.

In certain embodiments, the Cas nuclease is derived from Streptococcus Pyogenes Cas9 (NCBI Accession NO: WP_010922251). In certain embodiments, the Cas9 nuclease amino acid sequence comprises SEQ ID NO:1:

MDKKYSIGLDIGTNSVGWAVITDEYKVPSKKFKVLGNTDRHSIKKNLIGA
LLFDSGETAEATRLKRTARRRYTRRKNRICYLQEIFSNEMAKVDDSFFHR
LEESFLVEEDKKHERHPIFGNIVDEVAYHEKYPTIYHLRKKLVDSTDKAD
LRLIYLALAHMIKFRGHFLIEGDLNPDNSDVDKLFIQLVQTYNQLFEENP
INASGVDAKAILSARLSKSRRLENLIAQLPGEKKNGLFGNLIALSLGLTP
NFKSNFDLAEDAKLQLSKDTYDDDLDNLLAQIGDQYADLFLAAKNLSDAI
LLSDILRVNTEITKAPLSASMIKRYDEHHQDLTLLKALVRQQLPEKYKEI
FFDQSKNGYAGYIDGGASQEEFYKFIKPILEKMDGTEELLVKLNREDLLR
KQRTFDNGSIPHQIHLGELHAILRRQEDFYPFLKDNREKIEKILTFRIPY
YVGPLARGNSRFAWMTRKSEETITPWNFEEVVDKGASAQSFIERMTNFDK
NLPNEKVLPKHSLLYEYFTVYNELTKVKYVTEGMRKPAFLSGEQKKAIVD
LLFKTNRKVTVKQLKEDYFKKIECFDSVEISGVEDRFNASLGTYHDLLKI
IKDKDELDNEENEDILEDIVLTLTLFEDREMIEERLKTYAHLFDDKVMKQ
LKRRRYTGWGRLSRKLINGIRDKQSGKTILDFLKSDGFANRNFMQLIHDD
SLTFKEDIQKAQVSGQGDSLHEHIANLAGSPAIKKGILQTVKVVDELVKV
MGRHKPENIVIEMARENQTTQKGQKNSRERMKRIEEGIKELGSQILKEHP
VENTQLQNEKLYLYYLQNGRDMYVDQELDINRLSDYDVDHIVPQSFLKDD
SIDNKVLTRSDKNRGKSDNVPSEEVVKKMKNYWRQLLNAKLITQRKFDNL
TKAERGGLSELDKAGFIKRQLVETRQITKHVAQILDSRMNTKYDENDKLI
REVKVITLKSKLVSDFRKDFQFYKVREINNYHHAHDAYLNAVVGTALIKK
YPKLESEFVYGDYKVYDVRKMIAKSEQEIGKATAKYFFYSNIMNFFKTEI
TLANGEIRKRPLIETNGETGEIVWDKGRDFATVRKVLSMPQVNIVKKTEV
QTGGFSKESILPKRNSDKLIARKKDWDPKKYGGFDSPTVAYSVLVVAKVE
KGKSKKLKSVKELLGITIMERSSFEKNPIDFLEAKGYKEVKKDLIIKLPK
YSLFELENGRKRMLASAGELQKGNELALPSKYVNFLYLASHYEKLKGSPE
DNEQKQLFVEQHKHYLDEIIEQISEFSKRVILADANLDKVLSAYNKHRDK
PIREQAENIIHLFTLTNLGAPAAFKYFDTTIDRKRYTSTKEVLDATLIHQ
SITGLYETRIDLSQLGGD

In certain embodiments, the Cas9 nuclease amino acid sequence has at least about 85%, 86%, 87%, 88%, 89%, 90%, 91%, 92%, 93%, 94%, 95%, 96%, 97%, 98%, 99% or 100% sequence identity to SEQ ID NO:1.

In certain embodiments, the Cas nuclease is derived from Cas12a (NCBI Accession NO: WP_021736722). In certain embodiments, the Cas12a nuclease amino acid sequence comprises SEQ ID NO:2:

MTQFEGFTNLYQVSKTLRFELIPQGKTLKHIQEQGFIEEDKARNDHYKEL
KPIIDRIYKTYADQCLQLVQLDWENLSAAIDSYRKEKTEETRNALIEEQA
TYRNAIHDYFIGRTDNLTDAINKRHAEIYKGLFKAELFNGKVLKQLGTVT
TTEHENALLRSFDKFTTYFSGFYFNRKNVFSAEDISTAIPHRIVQDNFPK
FKENCHIFTRLITAVPSLREHFENVKKAIGIFVSTSIEEVESFPFYNQLL
TQTQIDLYNQLLGGISREAGTEKIKGLNEVLNLAIQKNDETAHIIASLPH
RFIPLFKQILSDRNTLSFILEEFKSDEEVIQSFCKYKTLLRNENVLETAE
ALFNELNSIDLTHIFISHKKLETISSALCDHWDTLRNALYERRISELTGK
ITKSAKEKVQRSLKHEDINLQEIISAAGKELSEAFKQKTSEILSHAHAAL
DQPLPTTLKKQEEKEILKSQLDSLLGLYHLLDWFAVDESNEVDPEFSARL
TGIKLEMEPSLSFYNKARNYATKKPYSVEKFKLNFQMPTLASGWDVNKEK
NNGAILFVKNGLYYLGIMPKQKGRYKALSFEPTEKTSEGFDKMYYDYFPD
AAKMIPKCSTQLKAVTAHFQTHTTPILLSNNFIEPLEITKEIYDLNNPEK
EPKKFQTAYAKKTGDQKGYREALCKWIDFTRDFLSKYTKTTSIDLSSLRP
SSQYKDLGEYYAELNPLLYHISFQRIAEKEIMDAVETGKLYLFQIYNKDF
AKGHHGKPNLHTLYWTGLFSPENLAKTSIKLNGQAELFYRPKSRMKRMAH
RLGEKMLNKKLKDQKTPIPDTLYQELYDYVNHRLSHDLSDEARALLPNVI
TKEVSHEIIKDRRFTSDKFFFHVPITLNYQAANSPSKFNQRVNAYLKEHP
ETPIIGIDRGERNLIYITVIDSTGKILEQRSLNTIQQFDYQKKLDNREKE
RVAARQAWSVVGTIKDLKQGYLSQVIHEIVDLMIHYQAVVVLENLNFGFK
SKRTGIAEKAVYQQFEKMLIDKLNCLVLKDYPAEKVGGVLNPYQLTDQFT
SFAKMGTQSGFLFYVPAPYTSKIDPLTGFVDPFVWKTIKNHESRKHFLEG
FDFLHYDVKTGDFILHFKMNRNLSFQRGLPGEMPAWDIVFEKNETQFDAK
GTPFIAGKRIVPVIENHRFTGRYRDLYPANELIALLEEKGIVFRDGSNIL
PKLLENDDSHAIDTMVALIRSVLQMRNSNAATGEDYINSPVRDLNGVCFD
SRFQNPEWPMDADANGAYHIALKGQLLLNHLKESKDLKLQNGISNQDWLA
YIQELRN

In certain embodiments, the Cas nuclease amino acid sequence has at least about 85%, 86%, 87%, 88%, 89%, 90%, 91%, 92%, 93%, 94%, 95%, 96%, 97%, 98%, 99% or 100% sequence identity to SEQ ID NO:2.

In certain embodiments, the retrotransposon RT is derived from retrotransposon LINE-1 ORF2p (e.g., see NCBI Accession NOs: 000370, 138588, S65824 and AAB59368). In certain embodiments, the ORF2p RT amino acid sequence comprises SEQ ID NO:3:

SIEKEGILPNSFYEASIILIPKPGRDTTKKENFRPISLMNIDAKILNKIL
ANRIQQHIKKLIHHDQVGFIPGMQGWFNIRKSINVIQHINRAKDKNHMII
SIDAEKAFDKIQQPFMLKTLNKLGIDGTYFKIIRAIYDKPTANIILNGQK
LEAFPLKTGTRQGCPLSPLLFNIVLEVLARAIRQEKEIKGIQLGKEEVKL
SLFADDMIVYLENPIVSAQNLLKLISNFSKVSGYKINVQKSQAFLYTNNR
QTESQIMGELPFTIASKRIKYLGIQL

In certain embodiments, the ORF2p RT amino acid sequence has as at least about 85%, 86%, 87%, 88%, 89%, 90%, 91%, 92%, 93%, 94%, 95%, 96%, 97%, 98%, 99% or 100% sequence identity to SEQ ID NO:3.

Thus, in certain embodiments, the present invention provides a recombinant polypeptide comprising a Cas nuclease amino acid sequence operably linked to an ORF2p RT amino acid sequence. In certain embodiments, the Cas nuclease has at least about 80% sequence identity to SEQ ID NO:1 or SEQ ID NO:2, and the OFR2p RT amino acid sequence has at least about 80% sequence identity to SEQ ID NO:3. In certain embodiments, the Cas nuclease amino acid sequence has at least about 95% sequence identity to SEQ ID NO:1 or SEQ ID NO:2, and the OFR2p RT amino acid sequence has at least about 95% sequence identity to SEQ ID NO:3. In certain embodiments, the Cas nuclease amino acid sequence has 100% sequence identity to SEQ ID NO: 1 or SEQ ID NO:2, and the OFR2p RT amino acid sequence has 100% sequence identity to SEQ ID NO:3.

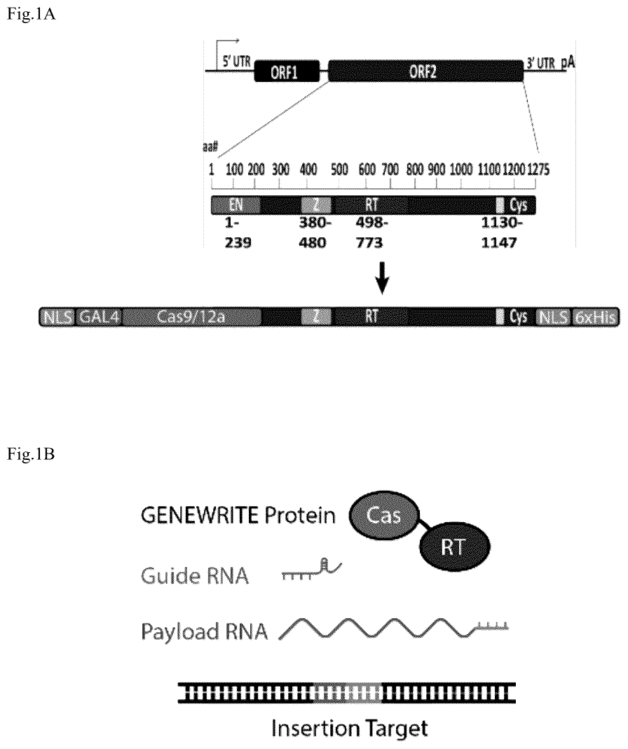

In certain embodiments, the recombinant polypeptide comprises a Cas nuclease amino acid sequence operably linked with an ORF2p RT amino acid sequence comprising amino acid residues from 498 to 773 of LINE-1 ORF2p (e.g., see NCBI Accession numbers 000370, 138588, S65824, AAB59368; an ORF2p sequence described herein, and FIG. 1).

In certain embodiments, the recombinant polypeptide comprises an ORF2p RT amino acid sequence, as well as other additional ORF2p amino acid sequences. For example, the recombinant peptide may comprise an ORF2p Cys domain sequence

(CWRGCGEIGTLLHCWWDC (SEQ ID NO: 16)),

an ORF2p Z domain sequence

(LIKKKREKNQIDTIKNDKGDITTDPTEIQTTIREYYKHLYANKLENLEEMDTFLDTYTLPRLNQ
EEVESLNRPITGSEIVAIINSLPTKKSPGPDGFTAEF (SEQ ID NO: 10)),

and/or an ORF2p Cryptic domain sequence

KNLTQSRSTTWKLNNLLLNDYWVHNEMKAEIKMFFETNENKDTTYQNLWDAFKAVCRGKFIALN
AYKRKQERSKIDTLTSQLKELEKQEQTHSKASRRQEITKIRAEL (SEQ ID NO: 11)).

In certain embodiments, the recombinant polypeptide comprises a Cas nuclease amino acid sequence operably linked with an ORF2p amino acid sequence, wherein the ORF2p amino acid sequence comprises residues 240 to 1275 of LINE-1 ORF2p (e.g., see NCBI Accession numbers 000370, 138588, S65824, AAB59368, SEQ ID NO:14 and FIG. 1) and does not comprise residues 1 to 239 of LINE-1 ORF2p (i.e., an ORF2p EN amino acid sequence). For example, in certain embodiments, the recombinant polypeptide comprises an amino acid sequence having at least about 85%, 86%, 87%, 88%, 89%, 90%, 91%, 92%, 93%, 94%, 95%, 96%, 97%, 98%, 99% or 100% sequence identity to SEQ ID NO:14.

(SEQ ID NO: 14)
KNLTQSRSTTWKLNNLLLNDYWVHNEMKAEIKMFFETNENKDTTYQNLWD
AFKAVCRGKFIALNAYKRKQERSKIDTLTSQLKELEKQEQTHSKASRRQE
ITKIRAELKEIETQKTLQKINESRSWFFERINKIDRPLARLIKKKREKNQ
IDTIKNDKGDITTDPTEIQTTIREYYKHLYANKLENLEEMDTFLDTYTLP
RLNQEEVESLNRPITGSEIVAIINSLPTKKSPGPDGFTAEFYQRYMEELV
PFLLKLFQSIEKEGILPNSFYEASIILIPKPGRDTTKKENFRPISLMNID
AKILNKILANRIQQHIKKLIHHDQVGFIPGMQGWFNIRKSINVIQHINRA
KDKNHMIISIDAEKAFDKIQQPFMLKTLNKLGIDGTYFKIIRAIYDKPTA
NIILNGQKLEAFPLKTGTRQGCPLSPLLFNIVLEVLARAIRQEKEIKGIQ
LGKEEVKLSLFADDMIVYLENPIVSAQNLLKLISNFSKVSGYKINVQKSQ
AFLYTNNRQTESQIMGELPFTIASKRIKYLGIQLTRDVKDLFKENYKPLL
KEIKEETNKWKNIPCSWVGRINIVKMAILPKVIYRFNAIPIKLPMTFFTE
LEKTTLKFIWNQKRARIAKSILSQKNKAGGITLPDFKLYYKATVTKTAWY
WYQNRDIDQWNRTEPSEIMPHIYNYLIFDKPEKNKQWGKDSLFNKWCWEN
WLAICRKLKLDPFLTPYTKINSRWIKDLNVKPKTIKTLEENLGITIQDIG
VGKDFMSKTPKAMATKDKIDKWDLIKLKSFCTAKETTIRVNRQPTTWEKI
FATYSSDKGLISRIYNELKQIYKKKTNNPIKKWAKDMNRHFSKEDIYAAK
KHMKKCSSSLAIREMQIKTTMRYHLTPVRMAIIKKSGNNRCWRGCGEIGT
LLHCWWDCKLVQPLWKSVWRFLRDLELEIPFDPAIPLLGIYPNEYKSCCY
KDTCTRMFIAALFTIAKTWNQPKCPTMIDWIKKMWHIYTMEYYAAIKNDE
FISFVGTWMKLETIILSKLSQEQKTKHRIFSLIGGN.

In certain embodiment, the ORF2p EN sequence (1-239) is fully or partially replaced by the Cas nuclease sequence. Thus, in certain embodiments, the ORF2p amino acid sequence does not comprise an ORF2p EN sequence (1-239). In certain other embodiments, the ORF2p amino acid sequence comprises a portion of ORF2p EN sequence, wherein the EN sequence is not functional.

In certain embodiments, a recombinant polypeptide described herein further comprises a DNA binding domain (DBD). In certain embodiments, the DBD is operably linked to the Cas nuclease amino acid sequence and the ORF2p RT amino acid sequence. In certain embodiments, the DNA binding domain in the recombinant polypeptide facilitates dimer formation or enhances the efficiency of retro-transposition and/or reverse transcription. In certain embodiments, the DBD comprises a GAL4 DBD amino acid sequence. Thus, certain embodiments provide, a recombinant polypeptide comprising the following operably linked amino acid sequences, listed in order from N-terminus to C-terminus: a GAL4 DNA binding domain (DBD) amino acid sequence, a Cas nuclease amino acid sequence and an ORF2p reverse transcriptase (RT) amino acid sequence. An exemplary GAL4 DBD amino acid sequence is:

(SEQ ID NO: 4)
MKLLSSIEQACDICRLKKLKCSKEKPKCAKCLKNNWECRYSPKTKRSPLT
RAHLTEVESRLERLEQLFLLIFPREDLDMILKMDSLQDIKALLTGLFVQD
NVNKDAVTDRLASVETDMPLTLRQHRISATSSSEESSNKGQRQL

In certain embodiments, the GAL4 DBD sequence has at least about 85%, 86%, 87%, 88%, 89%, 90%, 91%, 92%, 93%, 94%, 95%, 96%, 97%, 98%, 99% or 100% sequence identity to SEQ ID NO:4.

In certain embodiments, the recombinant polypeptide described herein further comprises one or more nuclear localization signal (NLS) sequences. In certain embodiments, the one or more nuclear localization signal (NLS) sequences are located at or near the N-terminus and/or C-terminus of the recombinant polypeptide. In certain embodiment, the one or more NLS sequences comprise an EGL13 NLS sequence of SEQ ID NO:5:

MSRRRKANPTKLSENAKKLAKEVEN

In certain embodiment, the one or more NLS sequences comprise a c-Myc NLS sequence of SEQ ID NO:6:

VPAAKRVKLD

In certain embodiments, the recombinant polypeptide described herein further comprises a tag sequence (e.g., 6× His tag (SEQ ID NO: 17)). In certain embodiments, the tag sequence can facilitate purification or detection and is located at the N-terminus and/or C-terminus of the recombinant polypeptide. In certain embodiments, the recombinant polypeptide is free of a tag sequence (e.g., 6× His tag (SEQ ID NO: 17)) at the N-terminus and/or C-terminus.

In certain embodiments, the recombinant polypeptide described herein further comprises a peptide linker located between different functional domains of the recombinant polypeptide. In certain embodiments, the peptide linker can serve as an inert protein bridge that confers flexibility and/or space for each different functional domain to function effectively. In certain embodiments, there is a peptide linker between the Cas nuclease amino acid sequence and ORF2p RT amino acid sequence. In certain embodiments, there is a peptide linker between the Gal4 DNA binding domain and Cas nuclease amino acid sequence. In certain embodiments, the peptide linker is a glycine peptide linker comprising 4, 5, 6, 7, 8, 9, 10, 11, 12 or more glycine residues, or a glycine serine peptide linker (e.g., GS, GGGGS (SEQ ID NO: 18) or (GGGGS)₃ (SEQ ID NO: 19)). In certain embodiments, the peptide linker comprises 4 continuous glycine residues (SEQ ID NO: 43). In certain embodiments, the peptide linker comprises 10 continuous glycine residues (SEQ ID NO: 44). In certain embodiments, the peptide liner comprises an amino acid sequence of SEQ ID NO: 15: TVAIPSTPPTPSPAIA.

In certain embodiments, the recombinant polypeptide comprises Cas nuclease amino acid sequence and ORF2p RT amino acid sequence operably linked in an orientation from N-terminus to C-terminus (Cas nuclease is located towards the N-terminus of the recombinant polypeptide and ORF2p RT is located towards the C-terminus).

In certain embodiments, the recombinant polypeptide comprises the following operably linked amino acid sequences, which are listed in order from N-terminus to C-terminus: a Cas nuclease sequence (e.g., Cas9 or Cas12a), an ORF2p Cryptic domain sequence, an ORF2p Z domain sequence, an ORF2p RT sequence and ORF2p Cys domain sequence.

In certain embodiments, the recombinant polypeptide comprises the following operably linked amino acid sequences, which are listed in order from N-terminus to C-terminus: Gal4 DBD, Cas nuclease (e.g., Cas9 or Cas12a), ORF2p RT, and ORF2p Cys domain.

In certain embodiments, the recombinant polypeptide comprises the following operably linked amino acid sequences, which are listed in order from N-terminus to C-terminus: Gal4 DBD, Cas nuclease (e.g., Cas9 or Cas12a), ORF2p Z domain, ORF2p RT, and ORF2p Cys.

In certain embodiments, the recombinant polypeptide comprises the following operably linked amino acid sequences, which are listed in order from N-terminus to C-terminus: Gal4 DBD, Cas nuclease (e.g., Cas9 or Cas12a), ORF2p Cryptic domain, ORF2p Z domain, ORF2p RT, and ORF2p Cys domain.

In certain embodiments, the recombinant polypeptide comprises the following operably linked amino acid sequences, which are listed in order from N-terminus to C-terminus: EGL13 NLS, Gal4 DBD, Cas nuclease (e.g., Cas9 or Cas12a), a glycine peptide linker, an ORF2p RT, and c-Myc NLS.

In certain embodiments, the recombinant polypeptide comprises the following operably linked amino acid sequences, which are listed in order from N-terminus to C-terminus: EGL13 NLS, Gal4 DBD, Cas nuclease (e.g., Cas9 or Cas12a), a glycine peptide linker, ORF2p Cryptic domain, ORF2p Z domain, ORF2p RT, ORF2p Cys domain and c-Myc NLS.

An exemplary Cas9-ORF2p (240-1275) recombinant polypeptide of the invention comprises SEQ ID NO:7:

MSRRRKANPTKLSENAKKLAKEVENMKLLSSIEQACDICRLKKLKCSKEK
PKCAKCLKNNWECRYSPKTKRSPLTRAHLTEVESRLERLEQLFLLIFPRE
DLDMILKMDSLQDIKALLTGLFVQDNVNKDAVTDRLASVETDMPLTLRQH
RISATSSSEESSNKGQRQLTVAIPSTPPTPSPAIAMDKKYSIGLDIGTNS
VGWAVITDEYKVPSKKFKVLGNTDRHSIKKNLIGALLFDSGETAEATRLK
RTARRRYTRRKNRICYLQEIFSNEMAKVDDSFFHRLEESFLVEEDKKHER
HPIFGNIVDEVAYHEKYPTIYHLRKKLVDSTDKADLRLIYLALAHMIKFR
GHFLIEGDLNPDNSDVDKLFIQLVQTYNQLFEENPINASGVDAKAILSAR
LSKSRRLENLIAQLPGEKKNGLFGNLIALSLGLTPNFKSNFDLAEDAKLQ
LSKDTYDDDLDNLLAQIGDQYADLFLAAKNLSDAILLSDILRVNTEITKA
PLSASMIKRYDEHHQDLTLLKALVRQQLPEKYKEIFFDQSKNGYAGYIDG
GASQEEFYKFIKPILEKMDGTEELLVKLNREDLLRKQRTFDNGSIPHQIH
LGELHAILRRQEDFYPFLKDNREKIEKILTFRIPYYVGPLARGNSRFAWM
TRKSEETITPWNFEEVVDKGASAQSFIERMTNFDKNLPNEKVLPKHSLLY
EYFTVYNELTKVKYVTEGMRKPAFLSGEQKKAIVDLLFKTNRKVTVKQLK
EDYFKKIECFDSVEISGVEDRFNASLGTYHDLLKIIKDKDFLDNEENEDI
LEDIVLTLTLFEDREMIEERLKTYAHLEDDKVMKQLKRRRYTGWGRLSRK
LINGIRDKQSGKTILDFLKSDGFANRNFMQLIHDDSLTFKEDIQKAQVSG
QGDSLHEHIANLAGSPAIKKGILQTVKVVDELVKVMGRHKPENIVIEMAR
ENQTTQKGQKNSRERMKRIEEGIKELGSQILKEHPVENTQLQNEKLYLYY
LQNGRDMYVDQELDINRLSDYDVDHIVPQSFLKDDSIDNKVLTRSDKNRG
KSDNVPSEEVVKKMKNYWRQLLNAKLITQRKFDNLTKAERGGLSELDKAG
FIKRQLVETRQITKHVAQILDSRMNTKYDENDKLIREVKVITLKSKLVSD
FRKDFQFYKVREINNYHHAHDAYLNAVVGTALIKKYPKLESEFVYGDYKV
YDVRKMIAKSEQEIGKATAKYFFYSNIMNFFKTEITLANGEIRKRPLIET
NGETGEIVWDKGRDFATVRKVLSMPQVNIVKKTEVQTGGFSKESILPKRN
SDKLIARKKDWDPKKYGGFDSPTVAYSVLVVAKVEKGKSKKLKSVKELLG
ITIMERSSFEKNPIDFLEAKGYKEVKKDLIIKLPKYSLFELENGRKRMLA
SAGELQKGNELALPSKYVNFLYLASHYEKLKGSPEDNEQKQLFVEQHKHY
LDEIIEQISEFSKRVILADANLDKVLSAYNKHRDKPIREQAENIIHLFTL
TNLGAPAAFKYFDTTIDRKRYTSTKEVLDATLIHQSITGLYETRIDLSQL
GGDGGGGGGGGGGKNLTQSRSTTWKLNNLLLNDYWVHNEMKAEIKMFFET
NENKDTTYQNLWDAFKAVCRGKFIALNAYKRKQERSKIDTLTSQLKELEK
QEQTHSKASRRQEITKIRAELKEIETQKTLQKINESRSWFFERINKIDRP
LARLIKKKREKNQIDTIKNDKGDITTDPTEIQTTIREYYKHLYANKLENL
EEMDTFLDTYTLPRLNQEEVESLNRPITGSEIVAIINSLPTKKSPGPDGF
TAEFYQRYMEELVPFLLKLFQSIEKEGILPNSFYEASIILIPKPGRDTTK
KENFRPISLMNIDAKILNKILANRIQQHIKKLIHHDQVGFIPGMQGWFNI
RKSINVIQHINRAKDKNHMIISIDAEKAFDKIQQPFMLKTLNKLGIDGTY
FKIIRAIYDKPTANIILNGQKLEAFPLKTGTRQGCPLSPLLFNIVLEVLA
RAIRQEKEIKGIQLGKEEVKLSLFADDMIVYLENPIVSAQNLLKLISNFS
KVSGYKINVQKSQAFLYTNNRQTESQIMGELPFTIASKRIKYLGIQLTRD
VKDLFKENYKPLLKEIKEETNKWKNIPCSWVGRINIVKMAILPKVIYRFN
AIPIKLPMTFFTELEKTTLKFIWNQKRARIAKSILSQKNKAGGITLPDFK
LYYKATVTKTAWYWYQNRDIDQWNRTEPSEIMPHIYNYLIFDKPEKNKQW
GKDSLFNKWCWENWLAICRKLKLDPFLTPYTKINSRWIKDLNVKPKTIKT
LEENLGITIQDIGVGKDFMSKTPKAMATKDKIDKWDLIKLKSFCTAKETT
IRVNRQPTTWEKIFATYSSDKGLISRIYNELKQIYKKKINNPIKKWAKDM
NRHFSKEDIYAAKKHMKKCSSSLAIREMQIKTTMRYHLTPVRMAIIKKSG
NNRCWRGCGEIGTLLHCWWDCKLVQPLWKSVWRFLRDLELEIPFDPAIPL
LGIYPNEYKSCCYKDTCTRMFIAALFTIAKTWNQPKCPTMIDWIKKMWHI
YTMEYYAAIKNDEFISFVGTWMKLETIILSKLSQEQKTKHRIFSLIGGNV
PAAKRVKLDHHHHHH

### Referring to SEQ ID NO:7:

Residues 1-25: N-terminal EGL-13 Nuclear Localization Signal (NLS)
Residues 26-169: GAL4 DNA Binding Domain
Residues 170-185: linker
Residues 186-1553: Cas9 Coding Sequence
Residues 1554-1563: 10× Glycine Bridge (SEQ ID NO: 44)
Residues 1564-2599: ORF2p (240-1275) (includes Cryptic, Z, RT, and Cys)
   1564-1671: Cryptic Domain
   1704-1804: Z Domain
   1822-2097: RT Domain
   2454-2471: Cys Domain
Residues 2600-2609: C-terminal c-Myc Nuclear Localization Signal (NLS)
Residues 2610-2615: 6× His tag (SEQ ID NO: 17)

An exemplary nucleic acid sequence, which encodes SEQ ID NO:7, is shown below:

(SEQ ID NO: 12)
ATGAGCCGCCGCCGCAAAGCGAACCCGACCAAACTGAGCGAAAACGCGAAAAAACTGGCGAAAG
AAGTGGAAAACATGAAGCTACTGTCTTCTATCGAACAAGCATGCGATATTTGCCGACTTAAAAA
GCTCAAGTGCTCCAAAGAAAAACCGAAGTGCGCCAAGTGTCTGAAGAACAACTGGGAGTGTCGC
TACTCTCCCAAAACCAAAAGGTCTCCGCTGACTAGGGCACATCTGACAGAAGTGGAATCAAGGC
TAGAAAGACTGGAACAGCTATTTCTACTGATTTTTCCTCGAGAAGACCTTGACATGATTTTGAA
AATGGATTCTTTACAGGATATAAAAGCATTGTTAACAGGATTATTTGTACAAGATAATGTGAAT
AAAGATGCCGTCACAGATAGATTGGCTTCAGTGGAGACTGATATGCCTCTAACATTGAGACAGC
ATAGAATAAGTGCGACATCATCATCGGAAGAGAGTAGTAACAAAGGTCAAAGACAGTTGACTGT
AGCGATTCCGTCGACACCACCTACTCCCTCTCCAGCGATCGCAATGGATAAAAAATACAGCATT
GGTCTGGACATTGGCACGAATAGCGTTGGTTGGGCAGTGATTACCGATGAATACAAAGTCCCGT
CGAAAAAATTCAAAGTGCTGGGTAACACCGATCGCCATAGCATTAAGAAAAACCTGATCGGTGC
GCTGCTGTTTGATTCTGGCGAAACCGCGGAAGCAACGCGTCTGAAACGTACCGCACGTCGCCGT
TACACGCGCCGTAAAAATCGTATTTGCTATCTGCAGGAAATCTTTAGCAACGAAATGGCGAAAG
TCGATGACTCATTTTTCCACCGCCTGGAAGAATCGTTTCTGGTGGAAGAAGATAAAAAACATGA
ACGTCACCCGATTTTCGGCAATATCGTTGATGAAGTCGCGTACCATGAAAAATATCCGACGATT
TACCACCTGCGTAAAAAACTGGTGGATTCTACCGACAAAGCCGATCTGCGCCTGATTTATCTGG
CACTGGCTCATATGATCAAATTTCGTGGTCACTTCCTGATTGAAGGCGACCTGAACCCGGATAA
TAGTGACGTCGATAAACTGTTTATTCAGCTGGTGCAAACCTATAATCAGCTGTTCGAAGAAAAC
CCGATCAATGCAAGTGGTGTTGATGCGAAAGCCATTCTGTCCGCTCGCCTGAGTAAATCCCGCC
GTCTGGAAAACCTGATTGCACAGCTGCCGGGTGAAAAGAAAAACGGTCTGTTTGGCAATCTGAT
CGCTCTGTCACTGGGCCTGACGCCGAACTTTAAATCGAATTTCGACCTGGCAGAAGATGCTAAA
CTGCAGCTGAGCAAAGATACCTACGATGACGATCTGGACAACCTGCTGGCGCAAATTGGCGACC
AGTATGCCGACCTGTTTCTGGCGGCCAAAAATCTGTCAGATGCCATTCTGCTGTCGGACATCCT
GCGCGTGAACACCGAAATCACGAAAGCGCCGCTGTCAGCCTCGATGATTAAACGCTACGATGAA
CATCACCAGGACCTGACCCTGCTGAAAGCACTGGTTCGTCAGCAACTGCCGGAAAAATACAAAG
AAATTTTCTTTGACCAAAGTAAAAATGGTTATGCAGGCTACATCGATGGCGGTGCTTCCCAGGA
AGAATTCTACAAATTCATCAAACCGATCCTGGAAAAAATGGATGGTACGGAAGAACTGCTGGTG
AAACTGAATCGTGAAGATCTGCTGCGTAAACAACGCACCTTTGACAACGGTAGCATTCCGCATC
AGATCCACCTGGGCGAACTGCATGCGATTCTGCGCCGTCAGGAAGATTTTTATCCGTTCCTGAA
AGACAACCGTGAAAAAATCGAAAAAATCCTGACGTTTCGCATCCCGTATTACGTTGGTCCGCTG
GCACGTGGTAATAGCCGCTTCGCATGGATGACCCGCAAATCTGAAGAAACCATTACGCCGTGGA
ACTTTGAAGAAGTGGTTGATAAAGGCGCAAGCGCTCAGTCTTTTATCGAACGTATGACCAATTT
CGATAAAAACCTGCCGAATGAAAAAGTGCTGCCGAAACATTCTCTGCTGTATGAATACTTTACC
GTTTACAACGAACTGACGAAAGTGAAATATGTTACCGAGGGTATGCGCAAACCGGCGTTTCTGA
GTGGCGAACAGAAAAAAGCCATTGTGGATCTGCTGTTCAAAACCAATCGTAAAGTTACGGTCAA
ACAGCTGAAAGAAGATTACTTCAAGAAAATTGAATGTTTCGACAGCGTGGAAATTTCTGGTGTT
GAAGATCGTTTCAACGCCTCTCTGGGCACCTATCATGACCTGCTGAAAATCATCAAAGACAAAG
ATTTTCTGGATAACGAAGAAAACGAAGACATTCTGGAAGATATCGTGCTGACCCTGACGCTGTT
CGAAGATCGTGAAATGATTGAAGAACGCCTGAAAACGTACGCACACCTGTTTGACGATAAAGTT
ATGAAACAGCTGAAACGCCGTCGCTATACCGGTTGGGGCCGTCTGAGCCGCAAACTGATTAATG
GTATCCGCGATAAACAATCAGGCAAAACGATTCTGGATTTCCTGAAATCGGACGGCTTTGCCAA
CCGTAATTTCATGCAGCTGATCCATGACGATTCCCTGACCTTTAAAGAAGACATTCAGAAAGCA
CAAGTGTCAGGTCAAGGCGATTCGCTGCATGAACACATTGCGAACCTGGCCGGTTCACCGGCTA
TCAAAAAAGGCATCCTGCAGACCGTGAAAGTCGTGGATGAACTGGTGAAAGTTATGGGTCGTCA
CAAACCGGAAAACATTGTTATCGAAATGGCGCGCGAAAATCAGACCACGCAAAAAGGCCAGAAA
AACTCGCGTGAACGCATGAAACGCATTGAAGAAGGTATCAAAGAACTGGGCAGCCAGATTCTGA
AAGAACATCCGGTCGAAAACACCCAGCTGCAAAATGAAAAACTGTACCTGTATTACCTGCAAAA
TGGTCGTGACATGTATGTGGATCAGGAACTGGACATCAACCGCCTGTCTGACTATGATGTCGAC
CACATTGTGCCGCAGAGCTTTCTGAAAGACGATTCTATCGATAACAAAGTTCTGACCCGTAGTG
ATAAAAACCGCGGCAAAAGCGACAATGTCCCGTCTGAAGAAGTIGTGAAGAAAATGAAAAACTA
CTGGCGTCAACTGCTGAATGCGAAACTGATTACGCAGCGTAAATTCGATAACCTGACCAAAGCG
GAACGCGGCGGTCTGTCCGAACTGGATAAAGCCGGTTTTATCAAACGTCAACTGGTTGAAACCC
GCCAGATTACGAAACATGTCGCCCAGATCCTGGATTCACGCATGAACACGAAATACGACGAAAA
CGATAAACTGATCCGTGAAGTCAAAGTGATCACCCTGAAAAGTAAACTGGTTTCCGATTTCCGT
AAAGACTTTCAGTTCTACAAAGTCCGCGAAATTAACAATTACCATCACGCACACGATGCTTATC
TGAATGCAGTGGTTGGTACCGCTCTGATCAAAAAATATCCGAAACTGGAAAGCGAATTTGTGTA
TGGCGATTACAAAGTCTATGACGTGCGCAAAATGATTGCGAAATCCGAACAGGAAATCGGCAAA
GCGACCGCCAAATACTTTTTCTATTCAAACATCATGAACTTTTTCAAAACCGAAATTACGCTGG
CAAATGGTGAAATTCGTAAACGCCCGCTGATCGAAACCAACGGTGAAACGGGCGAAATTGTGTG
GGATAAAGGCCGTGACTTCGCGACCGTTCGCAAAGTCCTGTCGATGCCGCAAGTGAATATCGTG
AAGAAAACCGAAGTGCAGACGGGCGGTTTTAGTAAAGAATCCATCCTGCCGAAACGTAACAGCG
ATAAACTGATTGCGCGCAAAAAAGATTGGGACCCGAAAAAATACGGCGGTTTTGATAGTCCGAC
GGTTGCATATTCCGTCCTGGTCGTGGCTAAAGTCGAAAAAGGTAAAAGTAAAAAACTGAAATCC
GTGAAAGAACTGCTGGGCATTACCATCATGGAACGTAGCTCTTTTGAGAAAAACCCGATTGACT
TCCTGGAAGCCAAAGGTTACAAAGAAGTGAAAAAAGATCTGATCATCAAACTGCCGAAATATAG
CCTGTTCGAACTGGAAAACGGCCGTAAACGCATGCTGGCATCTGCTGGTGAACTGCAGAAAGGC
AATGAACTGGCACTGCCGAGTAAATATGTTAACTTTCTGTACCTGGCTAGCCATTATGAAAAAC
TGAAAGGTTCTCCGGAAGATAACGAACAGAAACAACTGTTCGTCGAACAACATAAACACTACCT
GGATGAAATCATCGAACAGATCTCAGAATTCTCGAAACGCGTGATTCTGGCGGATGCCAATCTG
GACAAAGTTCTGAGCGCGTATAACAAACATCGTGATAAACCGATTCGCGAACAGGCCGAAAATA
TTATCCACCTGTTTACCCTGACGAACCTGGGCGCACCGGCAGCTTTTAAATACTTCGATACCAC
GATCGACCGTAAACGCTATACCTCAACGAAAGAAGTTCTGGATGCTACCCTGATTCATCAATCG
ATCACCGGTCTGTATGAAACGCGTATTGATCTGAGTCAGCTGGGCGGTGACGGTGGTGGTGGTG
GTGGTGGTGGTGGTGGTAAGAATCTCACTCAAAGCCGCTCAACTACATGGAAACTGAACAACCT
GCTCCTGAATGACTACTGGGTACATAACGAAATGAAGGCAGAAATAAAGATGTTCTTTGAAACC
AACGAGAACAAAGACACCACATACCAGAATCTCTGGGACGCATTCAAAGCAGTGTGTAGAGGGA
AATTTATAGCACTAAATGCCTACAAGAGAAAGCAGGAAAGATCCAAAATTGACACCCTAACATC
ACAATTAAAAGAACTAGAAAAGCAAGAGCAAACACATTCAAAAGCTAGCAGAAGGCAAGAAATA
ACTAAAATCAGAGCAGAACTGAAGGAAATAGAGACACAAAAAACCCTTCAAAAAATCAATGAAT
CCAGGAGCTGGTTTTTTGAAAGGATCAACAAAATTGATAGACCGCTAGCAAGACTAATAAAGAA
AAAGAGAGAGAAGAATCAAATAGACACAATAAAAAATGATAAAGGGGATATCACCACCGATCCC
ACAGAAATACAAACTACCATCAGAGAATACTACAAACACCTCTACGCAAATAAACTAGAAAATC
TAGAAGAAATGGATACATTCCTCGACACATACACTCTCCCAAGACTAAACCAGGAAGAAGTTGA
ATCTCTGAATAGACCAATAACAGGCTCTGAAATTGTGGCAATAATCAATAGTTTACCAACCAAA
AAGAGTCCAGGACCAGATGGATTCACAGCCGAATTCTACCAGAGGTACATGGAGGAACTGGTAC
CATTCCTTCTGAAACTATTCCAATCAATAGAAAAAGAGGGAATCCTCCCTAACTCATTTTATGA
GGCCAGCATCATTCTGATACCAAAGCCGGGCAGAGACACAACCAAAAAAGAGAATTTTAGACCA
ATATCCTTGATGAACATTGATGCAAAAATCCTCAATAAAATACTGGCAAACCGAATCCAGCAGC
ACATCAAAAAGCTTATCCACCATGATCAAGTGGGCTTCATCCCTGGGATGCAAGGCTGGTTCAA
TATACGCAAATCAATAAATGTAATCCAGCATATAAACAGAGCCAAAGACAAAAACCACATGATT
ATCTCAATAGATGCAGAAAAAGCCTTTGACAAAATTCAACAACCCTTCATGCTAAAAACTCTCA
ATAAATTAGGTATTGATGGGACGTATTTCAAAATAATAAGAGCTATCTATGACAAACCCACAGC
CAATATCATACTGAATGGGCAAAAACTGGAAGCATTCCCTTTGAAAACCGGCACAAGACAGGGA
TGCCCTCTCTCACCGCTCCTATTCAACATAGTGTTGGAAGTTCTGGCCAGGGCAATCAGGCAGG
AGAAGGAAATAAAGGGTATTCAATTAGGAAAAGAGGAAGTCAAATTGTCCCTGTTTGCAGATGA
CATGATTGTTTATCTAGAAAACCCCATCGTCTCAGCCCAAAATCTCCTTAAGCTGATAAGCAAC
TTCAGCAAAGTCTCAGGATACAAAATCAATGTACAAAAATCACAAGCATTCTTATACACCAACA
ACAGACAAACAGAGAGCCAGATCATGGGTGAACTCCCATTCACAATTGCTTCAAAGAGAATAAA
ATACCTAGGAATCCAACTTACAAGGGATGTGAAGGACCTCTTCAAGGAGAACTACAAACCACTG
CTCAAGGAAATAAAAGAGGAGACAAACAAATGGAAGAACATTCCATGCTCATGGGTAGGAAGAA
TCAATATCGTGAAAATGGCCATACTGCCCAAGGTAATTTACAGATTCAATGCCATCCCCATCAA
GCTACCAATGACTTTCTTCACAGAATTGGAAAAAACTACTTTAAAGTTCATATGGAACCAAAAA
AGAGCCCGCATTGCCAAGTCAATCCTAAGCCAAAAGAACAAAGCTGGAGGCATCACACTACCTG
ACTTCAAACTATACTACAAGGCTACAGTAACCAAAACAGCATGGTACTGGTACCAAAACAGAGA
TATAGATCAATGGAACAGAACAGAGCCCTCAGAAATAATGCCGCATATCTACAACTATCTGATC
TTTGACAAACCTGAGAAAAACAAGCAATGGGGAAAGGATTCCCTATTTAATAAATGGTGCTGGG
AAAACTGGCTAGCCATATGTAGAAAGCTGAAACTGGATCCCTTCCTTACACCTTATACAAAAAT
CAATTCAAGATGGATTAAAGATTTAAACGTTAAACCTAAAACCATAAAAACCCTAGAAGAAAAC
CTAGGCATTACCATTCAGGACATAGGCGTGGGCAAGGACTTCATGTCCAAAACACCAAAAGCAA
TGGCAACAAAAGACAAAATTGACAAATGGGATCTAATTAAACTAAAGAGCTTCTGCACAGCAAA
AGAAACTACCATCAGAGTGAACAGGCAACCTACAACATGGGAGAAAATTTTTGCAACCTACTCA
TCTGACAAAGGGCTAATATCCAGAATCTACAATGAACTCAAACAAATTTACAAGAAAAAAACAA
ACAACCCCATCAAAAAGTGGGCGAAGGACATGAACAGACACTTCTCAAAAGAAGACATTTATGC
AGCCAAAAAACACATGAAGAAATGCTCATCATCACTGGCCATCAGAGAAATGCAAATCAAAACC
ACTATGAGATATCATCTCACACCAGTTAGAATGGCAATCATTAAAAAGTCAGGAAACAACAGGT
GCTGGAGAGGATGCGGAGAAATAGGAACACTTTTACACTGTTGGTGGGACTGTAAACTAGTTCA
ACCATTGTGGAAGTCAGTGTGGCGATTCCTCAGGGATCTAGAACTAGAAATACCATTTGACCCA
GCCATCCCATTACTGGGTATATACCCAAATGAGTATAAATCATGCTGCTATAAAGACACATGCA
CACGTATGTTTATTGCGGCACTATTCACAATAGCAAAGACTTGGAACCAACCCAAATGTCCAAC
AATGATAGACTGGATTAAGAAAATGTGGCACATATACACCATGGAATACTATGCAGCCATAAAA
AATGATGAGTTCATATCCTTTGTAGGGACATGGATGAAATTGGAAACCATCATTCTCAGTAAAC
TATCGCAAGAACAAAAAACCAAACACCGCATATTCTCACTCATAGGTGGGAATGTTCCGGCGGC
GAAACGCGTGAAACTGGATCATCACCATCACCATCAC.

In certain embodiments, the recombinant polypeptide comprises an amino acid sequence having at least about 85%, 86%, 87%, 88%, 89%, 90%, 91%, 92%, 93%, 94%, 95%, 96%, 97%, 98%, 99% or 100% sequence identity to SEQ ID NO:7. In certain embodiments, the recombinant polypeptide consists of an amino acid sequence having at least about 85%, 86%, 87%, 88%, 89%, 90%, 91%, 92%, 93%, 94%, 95%, 96%, 97%, 98%, 99% or 100% sequence identity to SEQ ID NO:7. In certain embodiments, the recombinant polypeptide is encoded by a nucleic acid sequence having at least about 85%, 86%, 87%, 88%, 89%, 90%, 91%, 92%, 93%, 94%, 95%, 96%, 97%, 98%, 99% or 100% sequence identity to SEQ ID NO:12.

In certain embodiments, the recombinant polypeptide comprises the following operably linked amino acid sequences, which are listed in order from N-terminus to C-terminus: EGL13 NLS, Gal4 DBD, Cas12a nuclease, a glycine peptide linker, ORF2p Cryptic domain, ORF2p Z domain, ORF2p RT, ORF2p Cys domain and c-Myc NLS.

An exemplary Cas12a-ORF2p (240-1275) recombinant polypeptide comprises SEQ ID NO: 8:

MSRRRKANPTKLSENAKKLAKEVENMKLLSSIEQACDICRLKKLKCSKEK
PKCAKCLKNNWECRYSPKTKRSPLTRAHLTEVESRLERLEQLFLLIFPRE
DLDMILKMDSLQDIKALLTGLFVQDNVNKDAVTDRLASVETDMPLTLRQH
RISATSSSEESSNKGQRQLTVAIPSTPPTPSPAIAMTQFEGFTNLYQVSK
TLRFELIPQGKTLKHIQEQGFIEEDKARNDHYKELKPIIDRIYKTYADQC
LQLVQLDWENLSAAIDSYRKEKTEETRNALIEEQATYRNAIHDYFIGRTD
NLTDAINKRHAEIYKGLFKAELFNGKVLKQLGTVTTTEHENALLRSFDKF
TTYFSGFYENRKNVFSAEDISTAIPHRIVQDNFPKFKENCHIFTRLITAV
PSLREHFENVKKAIGIFVSTSIEEVFSFPFYNQLLTQTQIDLYNQLLGGI
SREAGTEKIKGLNEVLNLAIQKNDETAHIIASLPHRFIPLFKQILSDRNT
LSFILEEFKSDEEVIQSFCKYKTLLRNENVLETAEALFNELNSIDLTHIF
ISHKKLETISSALCDHWDTLRNALYERRISELTGKITKSAKEKVQRSLKH
EDINLQEIISAAGKELSEAFKQKTSEILSHAHAALDQPLPTTLKKQEEKE
ILKSQLDSLLGLYHLLDWFAVDESNEVDPEFSARLTGIKLEMEPSLSFYN
KARNYATKKPYSVEKFKLNFQMPTLASGWDVNKEKNNGAILFVKNGLYYL
GIMPKQKGRYKALSFEPTEKTSEGFDKMYYDYFPDAAKMIPKCSTQLKAV
TAHFQTHTTPILLSNNFIEPLEITKEIYDLNNPEKEPKKFQTAYAKKTGD
QKGYREALCKWIDFTRDFLSKYTKTTSIDLSSLRPSSQYKDLGEYYAELN
PLLYHISFQRIAEKEIMDAVETGKLYLFQIYNKDFAKGHHGKPNLHTLYW
TGLFSPENLAKTSIKLNGQAELFYRPKSRMKRMAHRLGEKMLNKKLKDQK
TPIPDTLYQELYDYVNHRLSHDLSDEARALLPNVITKEVSHEIIKDRRFT
SDKFFFHVPITLNYQAANSPSKENQRVNAYLKEHPETPIIGIDRGERNLI
YITVIDSTGKILEQRSLNTIQQFDYQKKLDNREKERVAARQAWSVVGTIK
DLKQGYLSQVIHEIVDLMIHYQAVVVLENLNFGFKSKRTGIAEKAVYQQF
EKMLIDKLNCLVLKDYPAEKVGGVLNPYQLTDQFTSFAKMGTQSGFLFYV
PAPYTSKIDPLTGFVDPFVWKTIKNHESRKHFLEGFDFLHYDVKTGDFIL
HFKMNRNLSFQRGLPGEMPAWDIVFEKNETQFDAKGTPFIAGKRIVPVIE
NHRFTGRYRDLYPANELIALLEEKGIVERDGSNILPKLLENDDSHAIDTM
VALIRSVLQMRNSNAATGEDYINSPVRDLNGVCFDSRFQNPEWPMDADAN
GAYHIALKGQLLLNHLKESKDLKLQNGISNQDWLAYIQELRNGGGGGGGG
GGKNLTQSRSTTWKLNNLLLNDYWVHNEMKAEIKMFFETNENKDTTYQNL
WDAFKAVCRGKFIALNAYKRKQERSKIDTLTSQLKELEKQEQTHSKASRR
QEITKIRAELKEIETQKTLQKINESRSWFFERINKIDRPLARLIKKKREK
NQIDTIKNDKGDITTDPTEIQTTIREYYKHLYANKLENLEEMDTFLDTYT
LPRLNQEEVESLNRPITGSEIVAIINSLPTKKSPGPDGFTAEFYQRYMEE
LVPFLLKLFQSIEKEGILPNSFYEASIILIPKPGRDTTKKENFRPISLMN
IDAKILNKILANRIQQHIKKLIHHDQVGFIPGMQGWENIRKSINVIQHIN
RAKDKNHMIISIDAEKAFDKIQQPFMLKTLNKLGIDGTYFKIIRAIYDKP
TANIILNGQKLEAFPLKTGTRQGCPLSPLLFNIVLEVLARAIRQEKEIKG
IQLGKEEVKLSLFADDMIVYLENPIVSAQNLLKLISNFSKVSGYKINVQK
SQAFLYTNNRQTESQIMGELPFTIASKRIKYLGIQLTRDVKDLFKENYKP
LLKEIKEETNKWKNIPCSWVGRINIVKMAILPKVIYRENAIPIKLPMTFF
TELEKTTLKFIWNQKRARIAKSILSQKNKAGGITLPDFKLYYKATVTKTA
WYWYQNRDIDQWNRTEPSEIMPHIYNYLIFDKPEKNKQWGKDSLFNKWCW
ENWLAICRKLKLDPFLTPYTKINSRWIKDLNVKPKTIKTLEENLGITIQD
IGVGKDFMSKTPKAMATKDKIDKWDLIKLKSFCTAKETTIRVNRQPTTWE
KIFATYSSDKGLISRIYNELKQIYKKKTNNPIKKWAKDMNRHFSKEDIYA
AKKHMKKCSSSLAIREMQIKTTMRYHLTPVRMAIIKKSGNNRCWRGCGEI
GTLLHCWWDCKLVQPLWKSVWRFLRDLELEIPFDPAIPLLGIYPNEYKSC
CYKDTCTRMFIAALFTIAKTWNQPKCPTMIDWIKKMWHIYTMEYYAAIKN
DEFISFVGTWMKLETIILSKLSQEQKTKHRIFSLIGGNVPAAKRVKLDHH
HHHH

Referring to SEQ ID NO:8:
Residues 1-25: N-terminal EGL-13 Nuclear Localization Signal (NLS)
Residues 26-169: GAL4 DNA Binding Domain
Residues 170-185: linker
Residues 186-1492: Cas12a/Cpf1 Coding Sequence
Residues 1493-1502: 10× Glycine Bridge (SEQ ID NO: 44)
Residues 1503-2538: ORF2p (240-1275) (includes Cryptic, Z, RT, and Cys)
   1503-1610: Cryptic Domain
   1643-1743: Z Domain
   1761-2036: RT Domain
   2393-2410: Cys Domain
Residues 2539-2548: C-terminal c-Myc Nuclear Localization Signal (NLS)
Residues 2549-2554: 6× His tag (SEQ ID NO: 17)

An exemplary nucleic acid sequence, which encodes SEQ ID NO:8, is shown below:

(SEQ ID NO: 13)
ATGAGCCGCCGCCGCAAAGCGAACCCGACCAAACTGAGCGAAAACGCGAAAAAACTGGCGAAAG
AAGTGGAAAACATGAAGCTACTGTCTTCTATCGAACAAGCATGCGATATTTGCCGACTTAAAAA
GCTCAAGTGCTCCAAAGAAAAACCGAAGTGCGCCAAGTGTCTGAAGAACAACTGGGAGTGTCGC
TACTCTCCCAAAACCAAAAGGTCTCCGCTGACTAGGGCACATCTGACAGAAGTGGAATCAAGGC
TAGAAAGACTGGAACAGCTATTTCTACTGATTTTTCCTCGAGAAGACCTTGACATGATTTTGAA
AATGGATTCTTTACAGGATATAAAAGCATTGTTAACAGGATTATTTGTACAAGATAATGTGAAT
AAAGATGCCGTCACAGATAGATTGGCTTCAGTGGAGACTGATATGCCTCTAACATTGAGACAGC
ATAGAATAAGTGCGACATCATCATCGGAAGAGAGTAGTAACAAAGGTCAAAGACAGTTGACTGT
AGCGATTCCGTCGACACCACCTACTCCCTCTCCAGCGATCGCAATGACCCAATTTGAAGGTTTT
ACCAATTTATACCAAGTTTCGAAGACCCTTCGTTTTGAACTGATTCCCCAAGGAAAAACACTCA
AACATATCCAGGAGCAAGGGTTCATTGAGGAGGATAAAGCTCGCAATGACCATTACAAAGAGTT
AAAACCAATCATTGACCGCATCTATAAGACTTATGCTGATCAATGTCTCCAACTGGTACAGCTT
GACTGGGAGAATCTATCTGCAGCCATAGACTCCTATCGTAAGGAAAAAACCGAAGAAACACGAA
ATGCGCTGATTGAGGAGCAAGCAACATATAGAAATGCGATTCATGACTACTTTATAGGTCGGAC
GGATAATCTGACAGATGCCATAAATAAGCGCCATGCTGAAATCTATAAAGGACTTTTTAAAGCT
GAACTTTTCAATGGAAAAGTTTTAAAGCAATTAGGGACCGTAACCACGACAGAACATGAAAATG
CTCTACTCCGTTCGTTTGACAAATTTACGACCTATTTTTCCGGCTTTTATGAAAACCGAAAAAA
TGTCTTTAGCGCTGAAGATATCAGCACGGCAATTCCCCATCGAATCGTCCAGGACAATTTCCCT
AAATTTAAGGAAAACTGCCATATTTTTACAAGATTGATAACCGCAGTTCCTTCTTTGCGGGAGC
ATTTTGAAAATGTCAAAAAGGCCATTGGAATCTTTGTTAGTACGTCTATTGAAGAAGTCTTTTC
CTTTCCCTTTTATAATCAACTTCTAACCCAAACGCAAATTGATCTTTATAATCAACTTCTCGGC
GGCATATCTAGGGAAGCAGGCACAGAAAAAATCAAGGGACTTAATGAAGTTCTCAATCTGGCTA
TCCAAAAAAATGATGAAACAGCCCATATAATCGCGTCCCTGCCGCATCGTTTTATTCCTCTTTT
TAAACAAATTCTTTCCGATCGAAATACGTTATCCTTTATTTTGGAAGAATTCAAAAGCGATGAG
GAAGTCATCCAATCCTTCTGCAAATATAAAACCCTCTTGAGAAACGAAAATGTACTGGAGACTG
CAGAAGCCCTTTTCAATGAATTAAATTCCATTGATTTGACTCATATCTTTATTTCCCATAAAAA
GTTAGAAACCATCTCTTCAGCGCTTTGTGACCATTGGGATACCTTGCGCAATGCACTTTACGAA
AGACGGATTTCTGAACTCACTGGCAAAATAACAAAAAGTGCCAAAGAAAAAGTTCAAAGGTCAT
TAAAACATGAGGATATAAATCTCCAAGAAATTATTTCTGCTGCAGGAAAAGAACTATCAGAAGC
ATTCAAACAAAAAACAAGTGAAATTCTTTCCCATGCCCATGCTGCACTTGACCAGCCTCTTCCC
ACAACATTAAAAAAACAGGAAGAAAAAGAAATCCTCAAATCACAGCTCGATTCGCTTTTAGGCC
TTTATCATCTTCTTGATTGGTTTGCTGTCGATGAAAGCAATGAAGTCGACCCAGAATTCTCAGC
ACGGCTGACAGGCATTAAACTAGAAATGGAACCAAGCCTTTCGTTTTATAATAAAGCAAGAAAT
TATGCGACAAAAAAGCCCTATTCGGTGGAAAAATTTAAATTGAATTTTCAAATGCCAACCCTTG
CCTCTGGTTGGGATGTCAATAAAGAAAAAAATAATGGAGCTATTTTATTCGTAAAAAATGGTCT
CTATTACCTTGGTATCATGCCTAAACAGAAGGGGCGCTATAAAGCCCTGTCTTTTGAGCCGACA
GAAAAAACATCAGAAGGATTCGATAAGATGTACTATGACTACTTCCCAGATGCCGCAAAAATGA
TTCCTAAGTGTTCCACTCAGCTAAAGGCTGTAACCGCTCATTTTCAAACTCATACCACCCCCAT
TCTTCTCTCAAATAATTTCATTGAACCTCTTGAAATCACAAAAGAAATTTATGACCTGAACAAT
CCTGAAAAGGAGCCTAAAAAGTTTCAAACGGCTTATGCAAAGAAGACAGGCGATCAAAAAGGCT
ATAGAGAAGCGCTTTGCAAATGGATTGACTTTACGCGGGATTTTCTCTCTAAATATACGAAAAC
AACTTCAATCGATTTATCTTCACTCCGCCCTTCTTCGCAATATAAAGATTTAGGGGAATATTAC
GCCGAACTGAATCCGCTTCTCTATCATATCTCCTTCCAACGAATTGCTGAAAAGGAAATCATGG
ATGCTGTAGAAACGGGAAAATTGTATCTGTTCCAAATCTACAATAAGGATTTTGCGAAGGGCCA
TCACGGGAAACCAAATCTCCACACCCTGTATTGGACAGGTCTCTTCAGTCCTGAAAACCTTGCG
AAAACCAGCATCAAACTTAATGGTCAAGCAGAATTGTTCTATCGACCTAAAAGCCGCATGAAGC
GGATGGCCCATCGTCTTGGGGAAAAAATGCTGAACAAAAAACTAAAGGACCAGAAGACACCGAT
TCCAGATACCCTCTACCAAGAACTGTACGATTATGTCAACCACCGGCTAAGCCATGATCTTTCC
GATGAAGCAAGGGCCCTGCTTCCAAATGTTATCACCAAAGAAGTCTCCCATGAAATTATAAAGG
ATCGGCGGTTTACTTCCGATAAATTTTTCTTCCATGTTCCCATTACACTGAATTATCAAGCAGC
CAATAGTCCCAGTAAATTCAACCAGCGTGTCAATGCCTACCTTAAGGAGCATCCGGAAACGCCC
ATCATTGGTATCGATCGTGGAGAACGCAATCTAATCTATATTACCGTCATTGACAGTACTGGGA
AAATTTTGGAGCAGCGTTCCCTGAATACCATCCAGCAATTTGACTACCAAAAAAAATTGGACAA
CAGGGAAAAAGAGCGTGTTGCCGCCCGTCAAGCCTGGTCCGTCGTCGGAACGATCAAAGACCTT
AAACAAGGCTACTTGTCACAGGTCATCCATGAAATTGTAGACCTGATGATTCATTACCAAGCTG
TTGTCGTCCTTGAAAACCTCAACTTCGGATTTAAATCAAAACGGACAGGCATTGCCGAAAAAGC
AGTCTACCAACAATTTGAAAAGATGCTAATAGATAAACTCAACTGTTTGGTTCTCAAAGATTAT
CCTGCTGAGAAAGTGGGAGGCGTCTTAAACCCGTATCAACTTACAGATCAGTTCACGAGCTTTG
CAAAAATGGGCACGCAAAGCGGCTTCCTTTTCTATGTACCGGCCCCTTATACCTCAAAGATTGA
TCCCCTGACTGGTTTTGTCGATCCCTTTGTATGGAAGACCATTAAAAATCATGAAAGTCGGAAG
CATTTCCTAGAAGGATTTGATTTCCTGCATTATGATGTCAAAACAGGTGATTTTATCCTCCATT
TTAAAATGAATCGGAATCTCTCTTTCCAGAGAGGGCTTCCTGGCTTCATGCCAGCTTGGGATAT
TGTTTTCGAAAAGAATGAAACCCAATTTGATGCAAAAGGGACGCCCTTCATTGCAGGAAAACGA
ATTGTTCCTGTAATCGAAAATCATCGTTTTACGGGTCGTTACAGAGACCTCTATCCCGCTAATG
AACTCATTGCCCTTCTGGAAGAAAAAGGCATTGTCTTTAGAGACGGAAGTAATATATTACCCAA
ACTTTTAGAAAATGATGATTCTCATGCAATTGATACGATGGTCGCCTTGATTCGCAGTGTACTC
CAAATGAGAAACAGCAATGCCGCAACGGGGGAAGACTACATCAACTCTCCCGTTAGGGATCTGA
ACGGGGTGTGTTTCGACAGTCGATTCCAAAATCCAGAATGGCCAATGGATGCGGATGCCAACGG
AGCTTATCATATTGCCTTAAAAGGGCAGCTTCTTCTGAACCACCTCAAAGAAAGCAAAGATCTG
AAATTACAAAACGGCATCAGCAACCAAGATTGGCTGGCCTACATTCAGGAACTGAGAAACGGTG
GTGGTGGTGGTGGTGGTGGTGGTGGTAAGAATCTCACTCAAAGCCGCTCAACTACATGGAAACT
GAACAACCTGCTCCTGAATGACTACTGGGTACATAACGAAATGAAGGCAGAAATAAAGATGTTC
TTTGAAACCAACGAGAACAAAGACACCACATACCAGAATCTCTGGGACGCATTCAAAGCAGTGT
GTAGAGGGAAATTTATAGCACTAAATGCCTACAAGAGAAAGCAGGAAAGATCCAAAATTGACAC
CCTAACATCACAATTAAAAGAACTAGAAAAGCAAGAGCAAACACATTCAAAAGCTAGCAGAAGG
CAAGAAATAACTAAAATCAGAGCAGAACTGAAGGAAATAGAGACACAAAAAACCCTTCAAAAAA
TCAATGAATCCAGGAGCTGGTTTTTTGAAAGGATCAACAAAATTGATAGACCGCTAGCAAGACT
AATAAAGAAAAAGAGAGAGAAGAATCAAATAGACACAATAAAAAATGATAAAGGGGATATCACC
ACCGATCCCACAGAAATACAAACTACCATCAGAGAATACTACAAACACCTCTACGCAAATAAAC
TAGAAAATCTAGAAGAAATGGATACATTCCTCGACACATACACTCTCCCAAGACTAAACCAGGA
AGAAGTTGAATCTCTGAATAGACCAATAACAGGCTCTGAAATTGTGGCAATAATCAATAGTTTA
CCAACCAAAAAGAGTCCAGGACCAGATGGATTCACAGCCGAATTCTACCAGAGGTACATGGAGG
AACTGGTACCATTCCTTCTGAAACTATTCCAATCAATAGAAAAAGAGGGAATCCTCCCTAACTC
ATTTTATGAGGCCAGCATCATTCTGATACCAAAGCCGGGCAGAGACACAACCAAAAAAGAGAAT
TTTAGACCAATATCCTTGATGAACATTGATGCAAAAATCCTCAATAAAATACTGGCAAACCGAA
TCCAGCAGCACATCAAAAAGCTTATCCACCATGATCAAGTGGGCTTCATCCCTGGGATGCAAGG
CTGGTTCAATATACGCAAATCAATAAATGTAATCCAGCATATAAACAGAGCCAAAGACAAAAAC
CACATGATTATCTCAATAGATGCAGAAAAAGCCTTTGACAAAATTCAACAACCCTTCATGCTAA
AAACTCTCAATAAATTAGGTATTGATGGGACGTATTTCAAAATAATAAGAGCTATCTATGACAA
ACCCACAGCCAATATCATACTGAATGGGCAAAAACTGGAAGCATTCCCTTTGAAAACCGGCACA
AGACAGGGATGCCCTCTCTCACCGCTCCTATTCAACATAGTGTTGGAAGTTCTGGCCAGGGCAA
TCAGGCAGGAGAAGGAAATAAAGGGTATTCAATTAGGAAAAGAGGAAGTCAAATTGTCCCTGTT
TGCAGATGACATGATTGTTTATCTAGAAAACCCCATCGTCTCAGCCCAAAATCTCCTTAAGCTG
ATAAGCAACTTCAGCAAAGTCTCAGGATACAAAATCAATGTACAAAAATCACAAGCATTCTTAT
ACACCAACAACAGACAAACAGAGAGCCAGATCATGGGTGAACTCCCATTCACAATTGCTTCAAA
GAGAATAAAATACCTAGGAATCCAACTTACAAGGGATGTGAAGGACCTCTTCAAGGAGAACTAC
AAACCACTGCTCAAGGAAATAAAAGAGGAGACAAACAAATGGAAGAACATTCCATGCTCATGGG
TAGGAAGAATCAATATCGTGAAAATGGCCATACTGCCCAAGGTAATTTACAGATTCAATGCCAT
CCCCATCAAGCTACCAATGACTTTCTTCACAGAATTGGAAAAAACTACTTTAAAGTTCATATGG
AACCAAAAAAGAGCCCGCATTGCCAAGTCAATCCTAAGCCAAAAGAACAAAGCTGGAGGCATCA
CACTACCTGACTTCAAACTATACTACAAGGCTACAGTAACCAAAACAGCATGGTACTGGTACCA
AAACAGAGATATAGATCAATGGAACAGAACAGAGCCCTCAGAAATAATGCCGCATATCTACAAC
TATCTGATCTTTGACAAACCTGAGAAAAACAAGCAATGGGGAAAGGATTCCCTATTTAATAAAT
GGTGCTGGGAAAACTGGCTAGCCATATGTAGAAAGCTGAAACTGGATCCCTTCCTTACACCTTA
TACAAAAATCAATTCAAGATGGATTAAAGATTTAAACGTTAAACCTAAAACCATAAAAACCCTA
GAAGAAAACCTAGGCATTACCATTCAGGACATAGGCGTGGGCAAGGACTTCATGTCCAAAACAC
CAAAAGCAATGGCAACAAAAGACAAAATTGACAAATGGGATCTAATTAAACTAAAGAGCTTCTG
CACAGCAAAAGAAACTACCATCAGAGTGAACAGGCAACCTACAACATGGGAGAAAATTTTTGCA
ACCTACTCATCTGACAAAGGGCTAATATCCAGAATCTACAATGAACTCAAACAAATTTACAAGA
AAAAAACAAACAACCCCATCAAAAAGTGGGCGAAGGACATGAACAGACACTTCTCAAAAGAAGA
CATTTATGCAGCCAAAAAACACATGAAGAAATGCTCATCATCACTGGCCATCAGAGAAATGCAA
ATCAAAACCACTATGAGATATCATCTCACACCAGTTAGAATGGCAATCATTAAAAAGTCAGGAA
ACAACAGGTGCTGGAGAGGATGCGGAGAAATAGGAACACTTTTACACTGTTGGTGGGACTGTAA
ACTAGTTCAACCATTGTGGAAGTCAGTGTGGCGATTCCTCAGGGATCTAGAACTAGAAATACCA
TTTGACCCAGCCATCCCATTACTGGGTATATACCCAAATGAGTATAAATCATGCTGCTATAAAG
ACACATGCACACGTATGTTTATTGCGGCACTATTCACAATAGCAAAGACTTGGAACCAACCCAA
ATGTCCAACAATGATAGACTGGATTAAGAAAATGTGGCACATATACACCATGGAATACTATGCA
GCCATAAAAAATGATGAGTTCATATCCTTTGTAGGGACATGGATGAAATTGGAAACCATCATTC
TCAGTAAACTATCGCAAGAACAAAAAACCAAACACCGCATATTCTCACTCATAGGTGGGAATGT
TCCGGCGGCGAAACGCGTGAAACTGGATCATCACCATCACCATCAC

In certain embodiments, the recombinant polypeptide comprises an amino acid sequence having at least about 85%, 86%, 87%, 88%, 89%, 90%, 91%, 92%, 93%, 94%, 95%, 96%, 97%, 98%, 99% or 100% sequence identity to SEQ ID NO:8. In certain embodiments, the recombinant polypeptide consists of an amino acid sequence having at least about 85%, 86%, 87%, 88%, 89%, 90%, 91%, 92%, 93%, 94%, 95%, 96%, 97%, 98%, 99% or 100% sequence identity to SEQ ID NO:8. In certain embodiments, the recombinant polypeptide is encoded by a nucleic acid sequence having at least about 85%, 86%, 87%, 88%, 89%, 90%, 91%, 92%, 93%, 94%, 95%, 96%, 97%, 98%, 99% or 100% sequence identity to SEQ ID NO:13.

In certain embodiments, a recombinant polypeptide described herein is capable of facilitating the integration of a sequence encoded by a payload RNA into a genomic target site.

In certain embodiments, the present invention provides a recombinant nucleic acid sequence encoding a recombinant polypeptide described herein. In certain embodiments, the nucleic acid sequence is codon-optimized for efficient expression in a eukaryotic cell.

In certain embodiments, the present invention provides an expression cassette comprising a nucleic acid sequence described herein.

In certain embodiments, the present invention provides a vector comprising an expression cassette described herein or a nucleic acid sequence described herein. In certain embodiments, the vector is a plasmid or a viral vector (e.g., lentiviral vector, adeno-associated virus vector, etc.) as non-limiting examples.

### Genome Editing System/Composition

In certain embodiments, the present invention provides a genome editing system comprising a targeted retrotransposon recombinant polypeptide described herein, a guide RNA and a payload RNA. In certain embodiments, the guide RNA and the payload RNA are two separate RNAs as shown in FIG. 1. In certain embodiments, guide RNA and payload RNA can be combined into a single RNA. In certain other embodiments, one or more of the components are provided as a vector. Thus, in certain embodiments, the genome editing system comprises a vector comprising a nucleic acid encoding the targeted retrotransposon recombinant polypeptide described herein. In certain embodiments, the genome editing system comprises a vector comprising a nucleic acid encoding the guide RNA. In certain embodiments, the genome editing system comprises a vector comprising a nucleic acid encoding the payload RNA.

Accordingly, certain embodiments of the invention provide a genome editing system comprising the following components: a guide RNA or a vector comprising a nucleic acid encoding the guide RNA; a payload RNA or a vector comprising a nucleic acid encoding the payload RNA; and a recombinant polypeptide described herein or a vector encoding a recombinant polypeptide described herein, and wherein the vector optionally further comprises a nucleic acid encoding ORF1p.

As described herein, the guide RNA confers sequence specificity/selectivity to the engineered polypeptide for targeted integration of a payload sequence. Specifically, the guide RNA (gRNA), designed to hybridize to a target genome sequence, complexes with the Cas nuclease portion of the recombinant polypeptide and directs cleavage at the desired cut/integration site (see, FIG. 1). gRNA design techniques are described herein and known in the art (see, e.g., U.S. Pat. Nos. 9,790,490; 9,840,702; 9,981,020; 10,106,820 and 10,240,145, which are incorporated by reference herein for all purposes).

As described herein, the payload RNA encodes a desired sequence to be integrated into the target genome. Subsequent to the creation of the DNA break (e.g., double stranded DNA break), the RT portion of the recombinant polypeptide uses the payload RNA as a template to reverse-transcribe the payload sequence for genomic integration. Methods for designing a payload RNA are described herein and known in the art. In certain embodiments, the payload sequence can be one or more coding and/or non-coding genetic/genomic sequence (e.g., a gene or fragment thereof, and/or a regulatory sequence such as a promoter). In certain embodiments, the payload sequence encodes TP53 (e.g., UniprotKB-P04637, GenBank accession X02469.1), UBE3A (e.g., UniProtKB-Q05086, GenBank accession X98031), NF-1 (e.g., UniProtKB-P21359, GenBank accession M89914), Merlin (e.g., UniProtKB-P35240, GenBank accession Z22664), CREBBP (e.g., UniProtKB-Q92793, GenBank accession U85962.3), or EP300 (e.g., UniProtKB-Q09472, GenBank accession U01877), or a fragment thereof. In certain embodiments, the payload sequence further encodes one or more selection markers (e.g., fluorescent protein or resistance marker such as puromycin resistance).

In certain embodiments, the payload sequence has a length of about Int to 20000 nt. In certain embodiments, the payload sequence has a length of about 50 nt, 60 nt, 70 nt, 80 nt, 90 nt, 100 nt, 200 nt, 300 nt, 400 nt, 500 nt, 600 nt, 700 nt, 800 nt, 900 nt, 1000 nt, 1500 nt, 2000 nt, 2500 nt, 3000 nt, 3500 nt, 4000 nt, 4500 nt, 5000 nt, 5500 nt, 6000 nt, 6500 nt, 7000 nt, 7500 nt, 8000 nt, 8500 nt, 9000 nt, 9500 nt, 10000 nt, 11000 nt, 12000 nt, 13000 nt, 14000 nt, 15000 nt, 16000 nt, 17000 nt, 18000 nt, 19000 nt or 20000 nt.

In certain embodiments, the payload sequence has a length of about 1000 nt to 20000 nt, or longer than 20000 nt.

In certain embodiments, the payload sequence has a length of at least about 50 nt, 60 nt, 70 nt, 80 nt, 90 nt, 100 nt, 200 nt, 300 nt, 400 nt, 500 nt, 600 nt, 700 nt, 800 nt, 900 nt, 1000 nt, 1500 nt, 2000 nt, 2500 nt, 3000 nt, 3500 nt, 4000 nt, 4500 nt, 5000 nt, 5500 nt, 6000 nt, 6500 nt, 7000 nt, 7500 nt, 8000 nt, 8500 nt, 9000 nt, 9500 nt, or 10000 nt.

In certain embodiments, the payload RNA comprises a 3′ homology sequence at its 3′ terminus, which can hybridize with a target sequence (e.g., hybridize with a target genome homology sequence; see, FIG. 1). In certain embodiments, the target genome homology sequence adjoins the target genome guide sequence (see, FIG. 1).

In certain embodiments, the payload RNA 3′ homology end has a length longer than 10 nt. In certain embodiments, the payload RNA 3′ homology end has a length longer than 20 nt. In certain embodiments, the payload RNA 3′ homology end has a length longer than 30 nt. In certain embodiments, the payload RNA 3′ homology end has a length longer than 40 nt. In certain embodiments, the payload RNA 3′ homology end has a length longer than 50 nt.

In certain embodiments, the payload RNA 3′ homology end has a length longer than 10 nt but shorter than 100 nt. In certain embodiments, the payload RNA 3′ homology end has a length longer than 20 nt but shorter than 100 nt. In certain embodiments, the payload RNA 3′ homology end has a length longer than 30 nt but shorter than 100 nt. In certain embodiments, the payload RNA 3′ homology end has a length longer than 40 nt but shorter than 100 nt. In certain embodiments, the payload RNA 3′ homology end has a length than about 50 nt but shorter than 100 nt.

In certain embodiments, the payload RNA 3′ homology end has a length of about 10-90 nt. In certain embodiments, the payload RNA 3′ homology end has a length of about 20-80 nt. In certain embodiments, the payload RNA 3′ homology end has a length of about 30-70 nt, for example, a length of about 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69 or 70 nt. In certain embodiments, the payload RNA 3′ homology end has a length of about 35-65 nt. In certain embodiments, the payload RNA 3′ homology end has a length of about 40-60 nt. In certain embodiments, the payload RNA 3′ homology end has a length of about 5-15, 10-20, 15-25, 20-30, 25-35, 30-40, 35-45, 40-50, 45-55, 50-60, 55-65, 60-70 or 65-75 nt. In certain embodiments, the payload RNA 3′ homology end has a length of about 30-50 nt. In certain embodiments, the payload RNA 3′ homology end has a length of about 40-50 nt. In certain embodiments, the payload RNA 3′ homology end has a length of about 40 nt. In certain embodiments, the payload RNA 3′ homology end has a length of about 45 nt. In certain embodiments, the payload RNA 3′ homology end has a length of about 50 nt. In certain embodiments, the payload RNA 3′ homology end has a length of at least about 30 nt. In certain embodiments, the payload RNA 3′ homology end has a length of at least about 40 nt. In certain embodiments, the payload RNA 3′ homology end has a length of at least about 50 nt.

In certain embodiments, the payload RNA 3′ homology end has at least about 85%, 86%, 87%, 88%, 89%, 90%, 91%, 92%, 93%, 94%, 95%, 96%, 97%, 98%, or 99% complementarity to the target genome homology sequence. In certain embodiments, the payload RNA 3′ homology end has 100% complementarity to the target genome homology sequence.

In certain embodiments, the payload RNA comprises an A, AA, AAA, AAAA, AAAAA, AAAAAA or AAAAAAA sequence at its 3′ terminus. In certain embodiments, the payload RNA comprises a long poly-A tail (e.g., 30 bp poly-A or longer (“30 bp poly-A” disclosed as SEQ ID NO: 41)) at its 3′ terminus. In certain embodiments, the payload RNA is free of any long poly-A tail (e.g., 30 bp poly-A or longer (“30 bp poly-A” disclosed as SEQ ID NO: 41)) at its 3′ terminus, for example, the payload RNA ends with a 3′ homology end sequence that is lacking a 30 bp or longer poly-A tail (“30 bp poly-A” disclosed as SEQ ID NO: 41). In certain embodiments, the payload RNA is free of any poly-A tail at its 3′ terminus.

In certain embodiments, the payload RNA at its 5′ terminus shares a homology sequence with a target genome sequence upstream of the integration cut site. Accordingly, in certain embodiments, the payload RNA comprises a 5′ homology sequence, which can hybridize with a target genome integration upstream sequence (e.g., see FIGS. 1B-1D, red, the target genome guide sequence).

In certain embodiments, the payload RNA 5′ homology sequence segment ends with a G or C at its own 3′ terminus. In other words, within the payload RNA 5′ homology sequence segment, in the direction from 5′ to 3′, the last nucleotide at the 3′ terminus is a G or C. Accordingly, in certain embodiments, the payload RNA 5′ homology sequence ends with a G. In certain embodiments, the payload RNA 5′ homology sequence ends with a C. In certain embodiments, the payload RNA 5′ homology sequence ends with an A. In certain embodiments, the payload RNA 5′ homology sequence ends with a U.

In certain embodiments, the payload RNA 5′ homology end has at least about 85%, 86%, 87%, 88%, 89%, 90%, 91%, 92%, 93%, 94%, 95%, 96%, 97%, 98%, or 99% complementarity to the target genome integration upstream sequence. In certain embodiments, the payload RNA 5′ homology end has 100% complementarity to the target genome integration upstream sequence.

In certain embodiments, the payload RNA 5′ homology end has a length longer than Int. In certain embodiments, the payload RNA 5′ homology end has a length longer than 2 nt. In certain embodiments, the payload RNA 5′ homology end has a length longer than 3 nt. In certain embodiments, the payload RNA 5′ homology end has a length longer than 4 nt. In certain embodiments, the payload RNA 5′ homology end has a length longer than 5 nt. In certain embodiments, the payload RNA 5′ homology end has a length longer than 10 nt. In certain embodiments, the payload RNA 5′ homology end has a length longer than 15 nt. In certain embodiments, the payload RNA 5′ homology end has a length longer than 20 nt. In certain embodiments, the payload RNA 5′ homology end has a length longer than 30 nt.

In certain embodiments, the payload RNA 5′ homology end has a length longer than 1 nt but shorter than 50 nt. In certain embodiments, the payload RNA 5′ homology end has a length longer than 2 nt but shorter than 50 nt. In certain embodiments, the payload RNA 5′ homology end has a length longer than 3 nt but shorter than 50 nt. In certain embodiments, the payload RNA 5′ homology end has a length longer than 4 nt but shorter than 50 nt. In certain embodiments, the payload RNA 5′ homology end has a length than about 5 nt but shorter than 50 nt. In certain embodiments, the payload RNA 5′ homology end has a length longer than 10 nt but shorter than 50 nt. In certain embodiments, the payload RNA 5′ homology end has a length longer than 15 nt but shorter than 50 nt. In certain embodiments, the payload RNA 5′ homology end has a length longer than 20 nt but shorter than 50 nt. In certain embodiments, the payload RNA 5′ homology end has a length longer than 25 nt but shorter than 50 nt.

In certain embodiments, the payload RNA 5′ homology end has a length of about 1-30 nt. In certain embodiments, the payload RNA 5′ homology end has a length of about 1-20 nt. In certain embodiments, the payload RNA 5′ homology end has a length of about 1-15 nt. In certain embodiments, the payload RNA 5′ homology end has a length of about 1-10 nt. In certain embodiments, the payload RNA 5′ homology end has a length of about 1-5 nt. In certain embodiments, the payload RNA 5′ homology end has a length of about 1-3 nt. In certain embodiments, the payload RNA 5′ homology end has a length of about 1-2 nt. In certain embodiments, the payload RNA 5′ homology end has a length of about 2-18 nt. In certain embodiments, the payload RNA 5′ homology end has a length of about 3-17 nt. In certain embodiments, the payload RNA 5′ homology end has a length of about 5-15 nt. In certain embodiments, the payload RNA 5′ homology end has a length of about 1-30 nt, for example, a length of about 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 nt. In certain embodiments, the payload RNA 5′ homology end has a length of Int. In certain embodiments, the payload RNA 5′ homology end has a length of 2 nt.

In certain embodiments, the payload RNA 5′ homology end has a length of at least about Int. In certain embodiments, the payload RNA 5′ homology end has a length of at least about 2 nt. In certain embodiments, the payload RNA 5′ homology end has a length of at least about 3 nt. In certain embodiments, the payload RNA 5′ homology end has a length of at least about 4 nt. In certain embodiments, the payload RNA 5′ homology end has a length of at least about 5 nt. In certain embodiments, the payload RNA 5′ homology end has a length of at least about 10 nt. In certain embodiments, the payload RNA 5′ homology end has a length of at least about 15 nt. In certain embodiments, the payload RNA 5′ homology end has a length of at least about 20 nt. In certain embodiments, the payload RNA 5′ homology end has a length of at least about 25 nt. In certain embodiments, the payload RNA 5′ homology end has a length of at least about 30 nt.

In certain embodiments, the genome editing system further comprises a LINE-1 ORF1p protein (NCBI Accession No: P11260). In certain embodiments, the ORF1p protein comprises SEQ ID NO:9:

MAKGKRKNPTNRNQDHSPSSERSTPTPPSPGHPNTTENLDPDLKTFLMMM
IEDIKKDFHKSLKDLQESTAKELQALKEKQENTAKQVMEMNKTILELKGE
VDTIKKTQSEATLEIETLGKRSGTIDASISNRIQEMEERISGAEDSIENI
DTTVKENTKCKRILTQNIQVIQDTMRRPNLRIIGIDENEDFQLKGPANIF
NKIIEENFPNIKKEMPMIIQEAYRTPNRLDQKRNSSRHIIIRTTNALNKD
RILKAVREKGQVTYKGRPIRITPDFSPETMKARRAWTDVIQTLREHKCQP
RLLYPAKLSITIDGETKVFHDKTKFTQYLSTNPALQRIITEKKQYKDGNH
ALEQPRK

In certain embodiments, the ORF1p protein comprises an amino acid sequence having at least about 85%, 86%, 87%, 88%, 89%, 90%, 91%, 92%, 93%, 94%, 95%, 96%, 97%, 98%, 99% or 100% sequence identity to SEQ ID NO:9.

In certain embodiments, the genome editing system comprises a vector comprising a nucleic acid sequence encoding an ORFp1 protein.

In certain embodiments, the genome editing system further comprises one or more non-homologous end joining (NHEJ) proteins. In certain embodiments, the one or more NHEJ proteins comprise LigD. In certain embodiments, the one or more NHEJ proteins comprise Ku. In certain embodiments, the genome editing system comprises LigD and Ku (e.g., B. subtilis YkoU (LigD) and B. subtilis YkoV (Ku)).

In certain embodiments, for delivery of the genome editing system described herein into a cell that has endogenous NHEJ proteins, the genome editing system does not comprise NHEJ proteins (e.g., LigD and Ku). In certain embodiments, for delivery of the genome editing system described herein into a cell that has endogenous NHEJ proteins, the genome editing system further comprises NHEJ proteins (e.g., LigD and Ku).

In certain embodiments, the one or more NHEJ proteins comprise YKU70, YKU80, DNL4/LIG4, LIF1, NEJ1, RAD50, MRE11, SIR2, SIR3, SIR4, XRS2, KU70/80, DNA-PKcs, XRCC4, Ligase IV, CLF, APLF, BRCA1, BRCA2, Artemis, ATM, PNKP, WRN, DNA pol, Aprataxin or XLF.

In certain embodiments, the genome editing system comprises a vector comprising a nucleic acid sequence encoding an NHEJ protein(s) (e.g., LigD and/or Ku).

In certain embodiments, the ORF1p, LigD, and/or Ku further comprise one or more nuclear localization signal (NLS) sequences. In certain embodiment, the one or more NLS sequence comprise an EGL13 or c-Myc NLS sequence. In certain embodiments, the ORF1p, LigD, and/or Ku further comprises a tag sequence (e.g., a 6×His tag (SEQ ID NO: 17)).

As described herein, in certain embodiments, the genome editing system comprises one or more vectors comprising nucleic acids encoding the recombinant polypeptide, ORF1p, LigD and/or Ku. In certain embodiments, the present invention provides a vector comprising nucleic acids encoding the Cas-ORF2p recombinant polypeptide and ORF1p polypeptide. In certain embodiment, the genome editing system comprises a vector comprising nucleic acids encoding LigD and Ku.

In certain embodiments, the genome editing system comprises a vector comprising a nucleic acid encoding a guide RNA. In certain embodiments, the genome editing system comprises a vector comprising a nucleic acid encoding a payload RNA. In certain embodiments, the genome editing system comprises a vector comprising nucleic acids encoding a guide RNA and a payload RNA.

In certain embodiments, one or more polypeptides in the genome editing system/composition are provided as isolated or purified polypeptides. Alternatively, one or more polypeptides in the genome editing system are provided by one or more vectors comprising nucleic acids encoding the one or more polypeptides. Accordingly, the one or more polypeptides in the genome editing system can be provided as isolated or purified polypeptide(s), vector(s) or any combination thereof.

In certain embodiment, the guide RNA and/or payload RNA in the genome editing system/composition are provided as isolated or purified RNA. In certain embodiments, the guide RNA and/or payload RNA are provided by one or more vectors. Accordingly, guide RNA and payload RNA can be provided as isolated or purified RNA, vector(s) or any combination thereof. Thus, polypeptide(s) and RNA(s) in the genome editing system described herein can be provided as isolated or purified form, vector(s), or any combination thereof. In certain embodiments, all components of the genome editing system described herein can be provided as one or more vector(s).

In certain embodiments, the components of the genome editing system are present in a single composition (e.g., further comprising a carrier). In certain embodiments, each component of the genome editing system is individually present in its own composition (e.g., further comprising a carrier). In certain embodiments, some components are present together in a composition while other components are individually formulated in separate compositions. In certain embodiments, the composition(s) is a pharmaceutical composition comprising a pharmaceutically acceptable carrier.

In certain embodiments, the present invention provides a kit comprising a genome editing system described herein (e.g., a recombinant polypeptide described herein (e.g., a Cas-ORF2p recombinant polypeptide) or a vector comprising a nucleic acid encoding recombinant polypeptide; a guide RNA or a vector comprising a nucleic acid encoding the guide RNA; a payload RNA or a vector comprising a nucleic acid encoding the payload RNA); packaging material; and instructions for editing a target genomic sequence in a cell by contacting the cell with the genome editing system under conditions suitable for the components of the system to enter the cell and edit the target genomic sequence.

In certain embodiments, the kit further comprises ORF1p or a vector comprising a nucleic acid encoding ORF1p. In certain embodiments, the kit further comprises one or more NHEJ proteins or a vector comprising nucleic acids encoding the one or more NHEJ proteins. In certain embodiments, the one or more NHEJ proteins comprise LigD and/or Ku.

In certain embodiments, the present invention provides a cell comprising a genome editing system described herein. In certain embodiments, the present invention provides a cell comprising a recombinant polypeptide described herein (e.g., a Cas-ORF2p recombinant polypeptide), a nucleic acid described herein and/or a vector described herein. Component(s) of the genome editing system described herein can be introduced into a cell via liposome, nanoparticle, electroporation, microinjection or other suitable methods such as deterministic mechanoporation (DMP) (Nano Lett. 2020 Feb. 12; 20 (2): 860-867). The recombinant polypeptide/other components of the genome editing system can be introduced indirectly via intracellular delivery/expression of a vector comprising a nucleic acid encoding the polypeptide of interest. Alternatively, the recombinant polypeptide/other component can be introduced directly as a protein via intracellular or intranuclear delivery. In certain embodiments, the genome editing system can be introduced as pre-assembled ribonucleoprotein particles (RNPs) into a cell. For example, a Cas-Orf2p recombinant protein can be mixed with RNA(s) to form pre-assembled RNPs prior to introduction into a cell. In certain embodiments, the present invention provides a cell that has been transformed (e.g., transfected or transduced) by one or more vectors described herein.

Delivery of protein, nucleic acids and vectors into cells are described herein and are known in the art. In certain embodiments, about 10 ng to 1000 mg, about 100 ng to 100 mg, about 1 μg to 10 mg, or about 10 ug to 1 mg of protein, RNA, vector or RNP are delivered into cells. In certain embodiments, the cell is a prokaryotic cell (e.g., a bacterial cell). In certain embodiments, the cell is a eukaryotic cell (e.g., a yeast cell). In certain embodiments, the cell is a plant cell. In certain embodiments, the cell is a non-mammalian cell (e.g., a fish cell). In certain embodiments, the cell is a mammalian cell (e.g., a mouse, rat, dog, cat, monkey, rabbit, hamster, horse, cow, sheep, pig, goat, or camelids cell). In certain embodiments, the cell is a human cell.

The genome editing system described herein may be used to edit a gene or genome within a cell. Alternatively, the system may be used to manipulate genetic sequences that are not present in a cell (e.g., in a test tube).

### Genome Editing Complex(es)

In certain embodiments, the present invention provides one or more genome editing complexes.

In certain embodiments, the present invention provides a first genome editing complex comprising a recombinant polypeptide described herein (e.g., a Cas-ORF2p recombinant polypeptide) and a guide RNA. In certain embodiments, the first genome editing complex is a protein-RNA complex (e.g., the Cas nuclease portion of the recombinant polypeptide complexes with the guide RNA).

In certain embodiments, the present invention provides a second genome editing complex comprising a recombinant polypeptide described herein (e.g., a Cas-ORF2p recombinant polypeptide), a guide RNA and a target genome sequence. In certain embodiments, the second genome editing complex is a protein-RNA-DNA complex (e.g., the Cas nuclease portion of the recombinant polypeptide complexes with the guide RNA and the target genome sequence). For example, following the formation of the first genome editing complex, the protein-RNA-DNA genome editing complex may assemble, leading to site-specific DNA cleavage of the target genome (e.g., at the target genome guide sequence).

In certain embodiments, the present invention provides a third genome editing complex comprising a recombinant polypeptide described herein (e.g., a Cas-ORF2p recombinant polypeptide), a payload RNA and a target genome sequence. In certain embodiments, the third genome editing complex is a protein-RNA-DNA complex (e.g., the RT portion of the recombinant polypeptide complexes with the payload RNA having a 3′ homology end which hybridizes with the target genome sequence, for example, at the target genome homology sequence). Following the site-specific DNA cleavage of the target genome sequence by the second genome editing complex, the third genome editing complex reverse transcribes the payload sequence for genomic integration.

The genome editing complex(es) described herein can assemble within a cell or in a cell-free manner within a test tube.

In certain embodiments, the present invention provides a payload RNA, wherein the payload RNA is capable of forming a protein-RNA-DNA genome editing complex described herein. In certain embodiments, the payload RNA does not encode an ORF2p sequence. In certain embodiments, the payload RNA does not encode an ORF1p sequence. In certain embodiments, the payload RNA does not encode Alu.

In certain embodiments, the payload RNA comprises a 3′ homology end, which can hybridize to a target genome sequence. In certain embodiments the payload RNA comprises a 3′ homology end as described herein (e.g., having a length of about 40-50 nt).

### Certain Genome Editing Methods

In certain embodiments, the present invention provides methods of using a recombinant polypeptide described herein (e.g., a Cas-ORF2p recombinant polypeptide). In certain embodiments, the present invention provides a genome editing method that comprises introducing into a cell a recombinant polypeptide described herein (e.g., a Cas-ORF2p recombinant polypeptide). In certain embodiments, the present invention provides a genome editing method that includes introducing into a cell a recombinant polypeptide described herein (e.g., a Cas-ORF2p recombinant polypeptide), a guide RNA and a payload RNA.

In certain embodiments, the present invention provides a genome editing method, which comprises forming a first, a second or a third genome editing complex as described herein. In certain embodiments, the genome editing method comprises forming a first and a second genome editing complexes as described herein. In certain embodiments, the genome editing method comprises forming a first, a second and a third genome editing complexes as described herein.

In certain embodiments, the genome editing methods described herein further comprise introducing into the cell ORF1p. In certain embodiments, the genome editing methods further comprise introducing into the cell one or more non-homologous end joining (NHEJ) proteins. In certain embodiments, the NHEJ proteins comprise LigD. In certain embodiments, the NHEJ proteins comprise Ku. In certain embodiments, the NHEJ proteins comprise LigD and Ku. In certain embodiments, ORF1p, LigD and Ku are introduced into the cell.

In certain embodiments, the genome editing method comprises introducing into a cell the genome editing system described herein.

Thus, certain embodiments of the invention provide a method of editing a target genomic sequence in a cell comprising contacting the cell with a genome editing system described herein under conditions suitable for the components of the system to enter the cell and edit the target genomic sequence. In certain embodiments, the genome editing system comprises a guide RNA or a vector comprising a nucleic acid encoding the guide RNA; a payload RNA or a vector comprising a nucleic acid encoding the payload RNA; and a recombinant polypeptide described herein (e.g., a Cas-ORF2p recombinant polypeptide) or a vector comprising a nucleic acid encoding the recombinant polypeptide. The components of the genome editing system can be provided as isolated or purified protein, RNA, a vector or any combination thereof. The components of the genome editing system can be introduced to a cell concurrently or sequentially. In certain embodiments, a recombinant polypeptide described herein (e.g., a Cas-ORF2p recombinant polypeptide) or a vector comprising a nucleic acid encoding the recombinant polypeptide is introduced first, followed by the introduction of guide RNA and payload RNA, and then followed by the optional introduction of NHEJ proteins. In certain embodiments the cell is contacted with the genome editing system in vitro. In certain embodiments the cell is contacted with the genome editing system in vivo.

In certain embodiments, the genome editing methods described herein comprises contacting a double-stranded DNA sequence with a recombinant polypeptide described herein (e.g., a Cas-ORF2p recombinant polypeptide), a gRNA and a payload RNA, thereby cutting the double-stranded DNA sequence, thereby forming a DNA-RNA duplex through the payload RNA 3′ homology end, thereby generating a cDNA strand complementary to the payload RNA template, thereby removing the payload RNA template, thereby repairing the modified DNA sequence, and thereby integrating the desired payload sequence into the genome.

Certain embodiments also provide a method of treating a disease or disorder in a mammal in need thereof, comprising administering to the mammal the genome editing system as described herein. The components of the genome editing system can be administered to the mammal concurrently or sequentially. Methods of administering nucleic acids (e.g., DNA/RNA), vectors and/or polypeptides to a mammal are known in the art (e.g., via intravenous administration) (see, e.g., Juliano, R L., Nucleic Acids Res. 2016 Aug. 19; 44 (14): 6518-48 and Nils Link, et al., Nucleic Acids Res. 2006; 34 (2): e16, which are incorporated by reference herein). In certain embodiments, the components are mixed together in a single composition. In certain other embodiments, one or more components are formulated individually into separate compositions. In certain embodiments, the genome editing system can be used to inactivate, correct, replace or introduce a coding and/or non-coding genomic sequence (e.g., a gene or fragment thereof, and/or a regulatory sequence such as a promoter) to treat a disease (e.g., hereditary disease, infectious disease, cancer, etc.). For example, the technology described herein is expected to be generally applicable for the correction of human diseases and syndromes caused by mutations or deletions of proteins and regulatory elements. Examples of diseases amenable to correction include, e.g., Angelman syndrome and Prader-Willi syndrome (UBE3A mutation or deletion), neurofibromatosis type I and II (NF-1 and Merlin mutation or deletion), Rubinstein-Taybi syndrome (CREBBP and/or EP300 mutation or deletion) or Li-Fraumeni syndrome and various cancers associated with deletion or mutation of TP53.

Certain embodiments provide a genome editing system as described herein for use in medical therapy.

Certain embodiments provide a genome editing system as described herein for use in the treatment of a disease or disorder in a mammal in need thereof.

Certain embodiments provide the use of a genome editing system as described herein for preparing a medicament for the treatment of a disease or disorder in a mammal in need thereof.

### Administration

Components of the genome editing system described herein (e.g., polypeptides or nucleic acids) can be formulated as pharmaceutical compositions and administered to a mammalian host, such as a human patient in a variety of forms adapted to the chosen route of administration, i.e., orally or parenterally, by intravenous, intramuscular, topical or subcutaneous routes.

Thus, the present polypeptides or nucleic acids may be systemically administered in combination with a pharmaceutically acceptable vehicle such as an inert diluent. Such compositions and preparations should contain at least 0.1% of polypeptides or nucleic acids. The percentage of the compositions and preparations may, of course, be varied and may conveniently be between about 2 to about 60% of the weight of a given unit dosage form. The amount of the polypeptides or nucleic acids in such therapeutically useful compositions is such that an effective dosage level will be obtained. Of course, any material used in preparing any unit dosage form should be pharmaceutically acceptable and substantially non-toxic in the amounts employed. In addition, the polypeptides or nucleic acids may be incorporated into sustained-release preparations and devices.

The polypeptides or nucleic acids may also be administered intravenously or intraperitoneally by infusion or injection. Solutions of the polypeptides or nucleic acids can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

The pharmaceutical dosage forms suitable for injection or infusion can include sterile aqueous solutions or dispersions or sterile powders comprising the polypeptides or nucleic acids which are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions are prepared by incorporating the polypeptides or nucleic acids in the required amount in the appropriate solvent with various of the other ingredients enumerated above, as required, followed by filter sterilization. In the case of sterile powders for the preparation of sterile injectable solutions, the preferred methods of preparation are vacuum drying and the freeze drying techniques, which yield a powder of the polypeptides or nucleic acids plus any additional desired ingredient present in the previously sterile-filtered solutions.

Useful dosages of the polypeptides or nucleic acids can be determined by comparing their in vitro activity, and in vivo activity in animal models. Methods for the extrapolation of effective dosages in mice, and other animals, to humans are known to the art; for example, see U.S. Pat. No. 4,938,949.

The amount of the polypeptides or nucleic acids, required for use in treatment will vary with the route of administration, the nature of the condition being treated and the age and condition of the patient and will be ultimately at the discretion of the attendant physician or clinician.

The desired dose may conveniently be presented in a single dose or as divided doses administered at appropriate intervals, for example, as two, three, four or more sub-doses per day. The sub-dose itself may be further divided, e.g., into a number of discrete loosely spaced administrations.

Components of the genome editing system as described herein can also be administered in combination with other therapeutic agents.

### Certain Definitions

The term “nucleic acid” and “polynucleotide” refers to deoxyribonucleotides or ribonucleotides and polymers thereof in either single- or double-stranded form, composed of monomers (nucleotides) containing a sugar, phosphate and a base which is either a purine or pyrimidine. Unless specifically limited, the term encompasses nucleic acids containing known analogs of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions) and complementary sequences as well as the sequence explicitly indicated. Specifically, degenerate codon substitutions may be achieved by generating sequences in which the third position of one or more selected (or all) codons is substituted with mixed-base and/or deoxyinosine residues. A “nucleic acid fragment” is a fraction of a given nucleic acid molecule. Deoxyribonucleic acid (DNA) in the majority of organisms is the genetic material while ribonucleic acid (RNA) is involved in the transfer of information contained within DNA into proteins. The term “nucleotide sequence” refers to a polymer of DNA or RNA that can be single- or double-stranded, optionally containing synthetic, non-natural or altered nucleotide bases capable of incorporation into DNA or RNA polymers. The terms “nucleic acid,” “nucleic acid molecule,” “nucleic acid fragment,” “nucleic acid sequence or segment,” or “polynucleotide” may also be used interchangeably with gene, cDNA, DNA and RNA encoded by a gene, e.g., genomic DNA, and even synthetic DNA sequences. The term also includes sequences that include any of the known base analogs of DNA and RNA.

“Naturally occurring” is used to describe an object that can be found in nature as distinct from being artificially produced. For example, a protein or nucleotide sequence present in an organism (including a virus), which can be isolated from a source in nature and which has not been intentionally modified by man in the laboratory, is naturally occurring.

A “variant” of a molecule is a sequence that is substantially similar to the sequence of the native molecule.

“Recombinant nucleic acid molecule” is a combination of nucleic acid sequences that are joined together using recombinant nucleic acid technology and procedures used to join together nucleic acid sequences as described, for example, in Sambrook and Russell (2001). As used herein, the term “recombinant nucleic acid,” e.g., “recombinant DNA sequence or segment” refers to a nucleic acid, e.g., to DNA, that has been derived or isolated from any appropriate cellular source, that may be subsequently chemically altered in vitro, so that its sequence is not naturally occurring, or corresponds to naturally occurring sequences that are not positioned as they would be positioned in a genome that has not been transformed with exogenous DNA. An example of preselected DNA “derived” from a source would be a DNA sequence that is identified as a useful fragment within a given organism, and which is then chemically synthesized in essentially pure form. An example of such DNA “isolated” from a source would be a useful DNA sequence that is excised or removed from said source by chemical means, e.g., by the use of restriction endonucleases, so that it can be further manipulated, e.g., amplified, for use in the invention, by the methodology of genetic engineering. The nucleic acid length units of nt and bp are used interchangeably herein.

Thus, recovery or isolation of a given fragment of DNA from a restriction digest can employ separation of the digest on polyacrylamide or agarose gel by electrophoresis, identification of the fragment of interest by comparison of its mobility versus that of marker DNA fragments of known molecular weight, removal of the gel section containing the desired fragment, and separation of the gel from DNA. Therefore, “recombinant DNA” includes completely synthetic DNA sequences, semi-synthetic DNA sequences, DNA sequences isolated from biological sources, and DNA sequences derived from RNA, as well as mixtures thereof.

The term “gene” is used broadly to refer to any segment of nucleic acid associated with a biological function. Thus, genes include coding sequences and/or the regulatory sequences required for their expression. For example, gene refers to a nucleic acid fragment that expresses mRNA, functional RNA, or specific protein, including regulatory sequences. Genes also include nonexpressed DNA segments that, for example, form recognition sequences for other proteins. Genes can be obtained from a variety of sources, including cloning from a source of interest or synthesizing from known or predicted sequence information, and may include sequences designed to have desired parameters. In addition, a “gene” or a “recombinant gene” refers to a nucleic acid molecule comprising an open reading frame and including at least about one exon and (optionally) an intron sequence. The term “intron” refers to a DNA sequence present in a given gene which is not translated into protein and is generally found between exons.

A “vector” is defined to include, inter alia, any plasmid, cosmid, phage or binary vector in double or single stranded linear or circular form which may or may not be self-transmissible or mobilizable, and which can transform prokaryotic or eukaryotic host either by integration into the cellular genome or exist extrachromosomally (e.g., autonomous replicating plasmid with an origin of replication).

“Expression cassette” as used herein means a DNA sequence capable of directing expression of a particular nucleotide sequence in an appropriate host cell, comprising a promoter operably linked to the nucleotide sequence of interest which is operably linked to termination signals. It also typically comprises sequences required for proper translation of the nucleotide sequence. The coding region usually codes for a protein of interest but may also code for a functional RNA of interest, for example antisense RNA or a nontranslated RNA, in the sense or antisense direction. The expression cassette comprising the nucleotide sequence of interest may be chimeric, meaning that at least about one of its components is heterologous with respect to at least about one of its other components. The expression cassette may also be one that is naturally occurring but has been obtained in a recombinant form useful for heterologous expression. The expression of the nucleotide sequence in the expression cassette may be under the control of a constitutive promoter or of an inducible promoter that initiates transcription only when the host cell is exposed to some particular external stimulus. In the case of a multicellular organism, the promoter can also be specific to a particular tissue or organ or stage of development.

Such expression cassettes will comprise the transcriptional initiation region of the invention linked to a nucleotide sequence of interest. Such an expression cassette is provided with a plurality of restriction sites for insertion of the gene of interest to be under the transcriptional regulation of the regulatory regions. The expression cassette may additionally contain selectable marker genes.

“Coding sequence” refers to a DNA or RNA sequence that codes for a specific amino acid sequence and excludes the non-coding sequences. It may constitute an “uninterrupted coding sequence”, i.e., lacking an intron, such as in a cDNA or it may include one or more introns bounded by appropriate splice junctions. An “intron” is a sequence of RNA which is contained in the primary transcript but which is removed through cleavage and re-ligation of the RNA within the cell to create the mature mRNA that can be translated into a protein.

The terms “open reading frame” and “ORF” refer to the amino acid sequence encoded between translation initiation and termination codons of a coding sequence. The terms “initiation codon” and “termination codon” refer to a unit of three adjacent nucleotides (‘codon’) in a coding sequence that specifies initiation and chain termination, respectively, of protein synthesis (mRNA translation).

“Operably-linked” nucleic acids refers to the association of nucleic acid sequences on single nucleic acid fragment so that the function of one is affected by the other, e.g., an arrangement of elements wherein the components so described are configured so as to perform their usual function. For example, a regulatory DNA sequence is said to be “operably linked to” or “associated with” a DNA sequence that codes for an RNA or a polypeptide if the two sequences are situated such that the regulatory DNA sequence affects expression of the coding DNA sequence (i.e., that the coding sequence or functional RNA is under the transcriptional control of the promoter). Coding sequences can be operably-linked to regulatory sequences in sense or antisense orientation. Control elements operably linked to a coding sequence are capable of effecting the expression of the coding sequence. The control elements need not be contiguous with the coding sequence, so long as they function to direct the expression thereof. Thus, for example, intervening untranslated yet transcribed sequences can be present between a promoter and the coding sequence and the promoter can still be considered “operably linked” to the coding sequence.

The term “amino acid” includes the residues of the natural amino acids (e.g., Ala, Arg, Asn, Asp, Cys, Glu, Gln, Gly, His, Hyl, Hyp, Ile, Leu, Lys, Met, Phe, Pro, Ser, Thr, Trp, Tyr, and Val) in D or L form, as well as unnatural amino acids (e.g., dehydroalanine, homoserine, phosphoserine, phosphothreonine, phosphotyrosine, hydroxyproline, gamma-carboxyglutamate; hippuric acid, octahydroindole-2-carboxylic acid, statine, 1,2,3,4,-tetrahydroisoquinoline-3-carboxylic acid, penicillamine, ornithine, citruline, α-methyl-alanine, para-benzoylphenylalanine, phenylglycine, propargylglycine, sarcosine, and tert-butylglycine). The term also comprises natural and unnatural amino acids bearing a conventional amino protecting group (e.g., acetyl or benzyloxycarbonyl), as well as natural and unnatural amino acids protected at the carboxy terminus (e.g., as a (C₁-C₆)alkyl, phenyl or benzyl ester or amide; or as an α-methylbenzyl amide). Other suitable amino and carboxy protecting groups are known to those skilled in the art (See for example, T. W. Greene, Protecting Groups In Organic Synthesis; Wiley: New York, 1981, and references cited therein) The term also comprises natural and unnatural amino acids bearing a cyclopropyl side chain or an ethyl side chain.

The terms “polypeptide” and “protein” are used interchangeably herein. A protein molecule may exist in a purified form or may exist in a non-native environment such as, for example, a transgenic host cell or bacteriophage. Fragments and variants of the disclosed proteins or partial-length proteins encoded thereby are also encompassed by the present invention. By “fragment” or “portion” is meant a full length or less than full length of the amino acid sequence of a protein.

By “portion” or “fragment,” as it relates to a nucleic acid molecule, sequence or segment of the invention, when it is linked to other sequences for expression, is meant a sequence having at least about 80 nucleotides, more preferably at least about 150 nucleotides, and still more preferably at least about 400 nucleotides. If not employed for expressing, a “portion” or “fragment” means at least about 9, preferably 12, more preferably 15, even more preferably at least about 20, consecutive nucleotides, e.g., probes and primers (oligonucleotides), corresponding to the nucleotide sequence of the nucleic acid molecules of the invention.

The invention encompasses isolated or substantially purified protein compositions. In the context of the present invention, an “isolated” or “purified” polypeptide is a polypeptide that exists apart from its native environment and is therefore not a product of nature. A polypeptide may exist in a purified form or may exist in a non-native environment such as, for example, a transgenic host cell. For example, an “isolated” or “purified” protein, or biologically active portion thereof, is substantially free of other cellular material, or culture medium when produced by recombinant techniques, or substantially free of chemical precursors or other chemicals when chemically synthesized. A protein that is substantially free of cellular material includes preparations of protein or polypeptide having less than about 30%, 20%, 10%, 5%, (by dry weight) of contaminating protein. When the protein of the invention, or biologically active portion thereof, is recombinantly produced, preferably culture medium represents less than about 30%, 20%, 10%, or 5% (by dry weight) of chemical precursors or non-protein-of-interest chemicals. Fragments and variants of the disclosed proteins or partial-length proteins encoded thereby are also encompassed by the present invention. By “fragment” or “portion” is meant a full length or less than full length of the amino acid sequence of, a polypeptide or protein.

The terms “introduce to a cell” and “introduction to a cell” refers to contacting a cell with a composition described herein for intracellular delivery or administration of the composition. The genome editing system/components of such a system can be provided as isolated or purified protein, RNA, a vector or any combination thereof. Thus, the methods of introduction can be a combination of delivery methods. For example, a polypeptide or an RNA can be introduced indirectly via intracellular delivery/expression of a vector comprising a nucleic acid encoding the recombinant polypeptide or the RNA. Non-limiting examples of vector delivery methods include transformation (electroporation, transfection or transduction), viral and non-viral based delivery, nanoparticle delivery, liposomal delivery, etc. Alternatively, polypeptide(s) and RNA can be introduced through the use of non-limiting examples of nanoparticles, liposomes, electroporation, microinjection, and gene gun, etc.

“Homology” refers to the percent identity between two polynucleotides or two polypeptide sequences. Two DNA or polypeptide sequences are “homologous” to each other when the sequences exhibit at least about 75% to 85% (including 75%, 76%, 77%, 78%, 79%, 80%, 81%, 82%, 83%, 84%, and 85%), at least about 90%, or at least about 95% to 99% (including 95%, 96%, 97%, 98%, 99%) contiguous sequence identity over a defined length of the sequences.

As used herein, “sequence identity” or “identity” in the context of two nucleic acid or polypeptide sequences makes reference to a specified percentage of residues in the two sequences that are the same when aligned for maximum correspondence over a specified comparison window, as measured by sequence comparison algorithms or by visual inspection. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity may be adjusted upwards to correct for the conservative nature of the substitution. Sequences that differ by such conservative substitutions are said to have “sequence similarity” or “similarity.” Means for making this adjustment are well known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., as implemented in the program PC/GENE (Intelligenetics, Mountain View, California).

As used herein, “comparison window” makes reference to a contiguous and specified segment of an amino acid or polynucleotide sequence, wherein the sequence in the comparison window may comprise additions or deletions (i.e., gaps) compared to the reference sequence (which does not comprise additions or deletions) for optimal alignment of the two sequences. Generally, the comparison window is at least about 20 contiguous amino acid residues or nucleotides in length, and optionally can be 30, 40, 50, 100, or longer.

As used herein, “percentage of sequence identity” means the value determined by comparing two optimally aligned sequences over a comparison window, wherein the portion of the polypeptide or polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity.

The term “substantial identity” of polynucleotide sequences means that a polynucleotide comprises a sequence that has at least about 70%, 71%, 72%, 73%, 74%, 75%, 76%, 77%, 78%, or 79%, at least about 80%, 81%, 82%, 83%, 84%, 85%, 86%, 87%, 88%, or 89%, at least about 90%, 91%, 92%, 93%, or 94%, and at least about 95%, 96%, 97%, 98%, or 99% sequence identity, compared to a reference sequence using one of the alignment programs described using standard parameters. One of skill in the art will recognize that these values can be appropriately adjusted to determine corresponding identity of proteins encoded by two nucleotide sequences by taking into account codon degeneracy, amino acid similarity, reading frame positioning, and the like. Substantial identity of amino acid sequences for these purposes normally means sequence identity of at least about 70%, at least about 80%, 90%, or at least about 95%.

The term “substantial identity” in the context of a peptide indicates that a peptide comprises a sequence with at least about 70%, 71%, 72%, 73%, 74%, 75%, 76%, 77%, 78%, or 79%, 80%, 81%, 82%, 83%, 84%, 85%, 86%, 87%, 88%, or 89%, at least about 90%, 91%, 92%, 93%, or 94%, or 95%, 96%, 97%, 98% or 99%, sequence identity to the reference sequence over a specified comparison window. An indication that two peptide sequences are substantially identical is that one peptide is immunologically reactive with antibodies raised against the second peptide. Thus, a peptide is substantially identical to a second peptide, for example, where the two peptides differ only by a conservative substitution.

For sequence comparison, typically one sequence acts as a reference sequence to which test sequences are compared. When using a sequence comparison algorithm, test and reference sequences are input into a computer, subsequence coordinates are designated if necessary, and sequence algorithm program parameters are designated. The sequence comparison algorithm then calculates the percent sequence identity or complementarity for the test sequence(s) relative to the reference sequence, based on the designated program parameters.

The terms “treat” and “treatment” refer to both therapeutic treatment and prophylactic or preventative measures, wherein the object is to prevent or decrease an undesired physiological change or disorder. For purposes of this invention, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those prone to have the condition or disorder or those in which the condition or disorder is to be prevented.

The invention will now be illustrated by the following non-limiting Examples.

### Example 1

### Targeted Retrotransposon-Mediated Genomic Integration of a Payload Sequence

The function of one exemplary embodiment of the GENEWRITE system was tested and verified in the bacterium Escherichia coli.

In this example, GENEWRITE proteins (amino acid sequences SEQ ID NO:7 or SEQ ID NO: 8) were designed to contain the following domains from N-terminus to C-terminus:
(1) A N-terminal EGL13 nuclear localization signal (NLS) (SEQ ID NO:5) (e.g., to aid in transport to the nucleus for human/mammalian cells);
(2) A Gal4 DNA binding domain (SEQ ID NO:4) (e.g., to facilitate dimer formation and enhances the efficiency of retrotransposition/reverse transcription);
(3) A linker (SEQ ID NO:15) between Gal4 DBD and Cas nuclease domain;
(4) A modified LINE-1 ORF2p amino acid sequence comprising:
   Cas9 (SEQ ID NO:1) or Cas12a (Cpf1) (SEQ ID NO:2), wherein the LINE-1 ORF2pEN domain was deleted (residues 1-239);
   10× glycine residues (SEQ ID NO: 44) (e.g., to serve as an inert protein bridge);
   the remainder of the ORF2p protein sequence from residues 240-1275 of LINE-1 ORF2p (SEQ ID NO:14)
(5) A C-terminal c-Myc NLS (SEQ ID NO:6) (e.g., to aid in nuclear delivery);
(6) a 6×His tag (SEQ ID NO: 17) at the C terminal end (e.g., to facilitate in vitro protein purification).

The DNA coding sequences of the GENEWRITE proteins (Cas9-ORF2pZRT 240-1275 SEQ ID NO:12 and Cas12a/ORF2pZRT 240-1275 SEQ ID NO:13) were designed in Vector NTI software (Thermo Fisher), and sequences were synthesized de novo by GENEWIZ Gene Synthesis Service (South Plainfield, NJ) and cloned into plasmid pUC57-kan.

An expression plasmid “pUC57-kan-ZRT” was constructed to express the GENEWRITE protein from a T7 promoter of the high-copy number plasmid pUC57-kan. In addition to this main protein, the GENEWRITE system is optionally aided by concomitant expression/presence of additional complementary proteins: ORF1p and enzymes enabling DNA repair by nonhomologous end joining (NHEJ). An expression plasmid “pUC57-kan-ORF1p” was constructed for expression of ORF1p. For NHEJ proteins, the Bacillus subtilis two-protein NHEJ system of Ku and LigD (Lee G, Et al. Proc Natl Acad Sci USA. 2018 Dec. 4; 115 (49): 12465-12470) was used in this example. Optimized versions of ORF1p, Ku, and LigD with addition of NLS signals and 6×His tags (SEQ ID NO: 17) were developed to aid in use for mammalian systems/in vitro purification.

A guide RNA directs the Cas component of the GENEWRITE protein to bind and cut at the desired integration location; and a payload RNA encodes the desired genetic payload to be reverse transcribed into the desired integration location. The 3′ end of the payload RNA was designed to satisfy requirements of reverse transcription by ORF2pRT for optimized integration efficiency (described below).

In this example, guide and payload RNAs were designed to integrate streptomycin 3′-adenyltransferase gene aadA, which confers resistance to the antibiotic spectinomycin, into the lac promoter existing in the high-copy number plasmid pUC57-kan-CRT within the host E. coli strain BL21-AI. BL21-AI expresses T7 polymerase upon addition of the sugar L-arabinose, which in turn results in expression of the GENEWRITE protein and optional expression of the modified ORF1p protein. In addition, the modified NHEJ proteins Ku and LigD were supplied from the medium copy number plasmid pZA31 under anhydrotetracycline (aTc) inducible control of the promoter PLtetO1 (Lee G, Et al. Proc Natl Acad Sci USA. 2018 Dec. 4; 115 (49): 12465-12470).

To generate the guide RNA, oligos were ordered from Integrated DNA Technologies (IDT) encoding an RNA targeting the lac promoter, with the guide RNA coding sequence placed under control of a T7 promoter. Guide RNAs were produced through in vitro transcription with T7 polymerase (T7 Megascript Kit, Thermo Fisher Scientific). We then digest the reaction with TURBO DNAse (Thermo Fisher) and purify the resulting RNAs via phenol chloroform extraction followed by ethanol precipitation.

To produce payload RNAs, oligos were ordered from IDT to amplify the aadA gene from the plasmid pTKRED (Kuhlman, Et al. Nucleic Acids Res. 2010 April; 38(6):e92), with the oligos including a T7 promoter. After PCR amplification, RNAs were produced and purified in vitro as described above.

BL21-AI carrying pUC57-kan-Cas9-ORF2pZRT and pZA31-NHEJ was induced by addition of 0.1% w/v L-arabinose directly to the culture. After the arabinose was fully dissolved, the culture was immediately harvested to be prepared for electroporation. The induction of GENEWRITE protein expression prior to electroporation of the guide and payload RNAs should be short to limit GENEWRITE protein's toxicity to E. coli. The culture was washed 3× in ice cold 10% glycerol. For each electroporation reaction, 100 μl of prepared cells was mixed with ~10 μg guide RNA targeting the lac promoter in the chromosome and on the pUC57-kan plasmid, as well as 2.6 μg payload RNA encoding the aadA spectinomycin resistance gene. The mixture was placed into a 0.1 cm gap electroporation cuvette (USA Scientific) and electroporated using a Bio-Rad Micropulser Electroporator at 1 kV. Cells were mixed with 1 ml SOB medium including 2 mM isopropyl-β-D-thiogalactoside (IPTG) and 100 ng/ml anhydrotetracycline (aTc) to induce expression of B. subtilis NHEJ enzymes. Cells were allowed to recover ~24 hours in a 37° C. shaking water bath (New Brunswick C76). Cultures were then centrifuged and excess medium removed. Cells were resuspended in remaining medium and plated on LB agar plates containing 100 μg/ml spectinomycin, 2 mM IPTG, 100 ng/ml aTc, and 0.5% glucose. Plates were allowed to incubate 37° C. for 24 hours, and the number of spectinomycin resistant colonies counted.

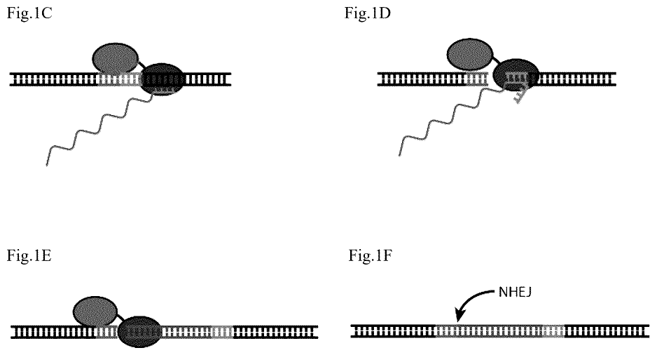

To verify site specific integration at the lac promoter, PCR was performed using pairs of oligos where one primer bound to the native lac operon sequence, while the other primer bound within the aadA sequence. Consequently, PCR product will only be produced upon successful integration and creation of lac-aadA junctions. Primers were designed to produce a 300 bp amplicon from the 5′ lac-aadA junction and a 400 bp amplicon from the 3′ lac-aadA junction. The result of PCR verification of four putatively successful integrants is shown in FIG. 2. Production of appropriately sized PCR amplicons illustrates site specific integration of aadA at the intended integration site at the lac promoter, and this result was verified by sequencing. To verify the generalizability of the method, similar attempts to integrate aadA into the medium copy number plasmid pZA31 were conducted, and with both variants of the GENEWRITE protein (either Cas9 or Cpf1), with similar success.

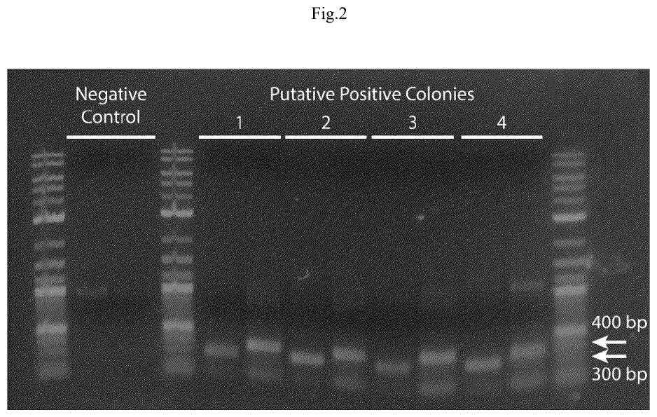

The sequence requirements of the payload RNA were explored to optimize integration efficiency. In order to facilitate TPRT, the 3′ end of the payload RNA should be designed to hybridize with the DNA adjacent to the cut generated by the Cas enzyme. To determine the optimal length of homology between the target site and the payload RNA, a family of six identical payload RNAs were generated, differing only in the length of homology to the integration site at the 3′ end, from 0 to 50 bp in 10 bp increments. In addition, another family of six payload RNAs with the same range of 0-50 bp homology were generated, but also including a 30 bp long poly-A tract (SEQ ID NO: 41) at the 3′ end; such a poly-A tract has previously been shown to be required for retrotransposition of the wildtype LINE-1 (Doucet A J, Et al. Mol Cell. 2015 Dec. 3; 60 (5): 728-741). The results are shown in FIG. 3 and illustrate that the optimal payload RNA design includes a 40 bp or 50 bp long 3′ homology region with the target site and without an additional 30 bp poly(A) tract (SEQ ID NO: 41) in this example. In a similar fashion, it is shown that concomitant expression of NHEJ enzymes facilitate integration in E. coli, and that simultaneous expression of ORF1p facilitates integration efficiency in E. coli.

### Example 2

### Successful Genomic Integration of Large Gene Sized Payload

As described herein, we have developed a genome editing tool termed GENome Engineering With RNA-Integrating Targeted Endonucleases, or GENEWRITE. GENEWRITE couples the targetability of CRISPR-Cas endonucleases with the ability to integrate large genetic constructs into the genome using the reverse transcriptase activity of the human retrotransposon LINE-1.

CRISPR-Cas endonucleases. Discovered as a bacterial immune system against foreign genetic elements such as phages, CRISPR (Clustered Regularly Interspaced Short Palindromic Repeats) Associated Proteins (Cas) are endonucleases that target and cleave DNA sequences based upon their homology with a “guide RNA”. Consequently, by providing an engineered “single-guide” RNA (sgRNA), Cas enzymes can be targeted to cleave any desired sequence. This flexibility in gene editing by CRISPR-Cas endonucleases has revolutionized genome editing in a wide variety of organisms and its application to clinical therapeutics.

Genome editing with CRISPR-Cas and its limitations. Despite their flexibility and ease of use, the repertoire of genome editing modalities that CRISPR/Cas systems allow remains limited. Knockout or point mutants can be generated relatively easily by targeting Cas cleavage to coding or control regions of the genome. The cell must repair such cuts to survive, for example, by the nonhomologous end joining (NHEJ) repair machinery. An additional editing modality is to introduce novel sequences to the genome through Homology Directed Repair (HDR), where a DNA fragment with ends homologous to the sequences flanking the cut site and containing the desired sequence to be inserted is introduced to the cell along with the Cas-sgRNA ribonucleoprotein (RNP) complexes. After cleavage, the fragment is then used to repair the cut by the cell's homologous recombination repair machinery, resulting in its integration. However, HDR remains inefficient and difficult to accomplish, particularly for gene-sized or larger [≥~1 kilobase pair (kbp)] fragments. A primary reason for this difficulty is that for HDR to be successful, NHEJ, the primary repair mechanism for DNA repair in advanced eukaryotic cells, must be suppressed. Because of this limitation, any clinical or scientific applications that require the site-specific addition of large genetic constructs remain extremely difficult or inaccessible.

The human retrotransposon LINE-1. LINE-1 (Long Interspersed Nuclear Element) is the sole remaining active autonomous retrotransposon in humans, with ~500,000 integrants and making up ~17% of the human genome. Of these, approximately 100 LINE-1 copies remain active, or “hot”, per individual. The complete LINE-1 sequence is ~6 kbp long, containing the two genes ORF1 (~1 kbp) and ORF2 (~4 kbp) which encode the proteins ORF1p and ORF2p respectively. Both proteins are required for retrotransposition in humans. ORF2p includes domains with endonuclease (EN) and reverse transcriptase (RT) domains. The primary function of ORF1p appears to be nucleic acid chaperone activity. Additionally, most hot L1 elements include an ~100 bp poly(A) tract (SEQ ID NO: 42) at the 3′ end after the ORF2 coding sequence.

The success of LINE-1 in replicating itself in the human genome is a consequence of its weak sequence requirements for retrotransposition using a mechanism called target primed reverse transcription (TPRT). After transcription and translation of LINE-1, ORF1p and ORF2p bind preferentially in cis to their encoding RNA, and the resulting ribonucleoprotein particle preferentially acts upon TA-rich DNA. ORF2p EN nicks the DNA at the degenerate consensus sequence 5′-TTTT/A-3′, and variants, with the cut occurring at the TpA bond. The 3′ poly(A) tract of the LINE-1 mRNA hybridizes with the cut site, and ORF2p RT uses this hybridization and exposed free 3′ hydroxyl group as a primer for reverse transcription, generating a new CDNA copy of LINE-1 at the new site.

As describe herein, we have developed a technique for targeting LINE-1 insertions to specific genomic loci. GENEWRITE works with the cell's natural DNA repair mechanisms to site-specifically integrate large genetic payloads. This contrasts with existing HDR technologies, where cells' dominant NHEJ repair mechanisms must be suppressed.

To increase the sequence specificity of ORF2p, we have replaced the promiscuous ORF2p endonuclease (EN) domain with targetable Cas endonucleases (Cas9 or Cas12a/Cpf1; FIG. 1A). Consequently, cleavage is programmable to specific locations in the genome by supplying the Cas-ORF2p protein with an appropriate guide RNA. Furthermore, we provide Cas-ORF2p with an additional substrate RNA for reverse transcription (FIG. 1B). This RNA carries any desired payload with a 3′ end homologous to the cut site. After cleavage of the target site by Cas (FIG. 1C), the 3′ end of the payload RNA hybridizes with the exposed target DNA (FIG. 1D) to prime TPRT and initiate reverse transcription (FIG. 1E). After reverse transcription, and as with native LINE-1 retrotransposition, the payload RNA is removed and the second strand is synthesized. Finally, the remaining exposed end of the original cut is sealed by NHEJ repair mechanisms, completing the insertion (FIG. 1F). In this way, we have added WRITE functionality to CRISPR-Cas systems, allowing for the active reverse transcription of arbitrary payloads into the genome at any specific locus.

Additional features of GENEWRITE may include the addition of nuclear localization signals to enhance transport of proteins and ribonucleoprotein complexes to the nucleus (NLS (green), FIG. 1A). As one example, we also add an N-terminal GAL4 domain (GAL4 (red), FIG. 1A) to enhance dimerization of ORF2p to facilitate retrotransposition.

A GENEWRITE protein described in Example 1 was/is used in the experiments described below, unless indicated otherwise.

GENEWRITE is Expressed in E. coli and is Lethal in the Absence of NHEJ

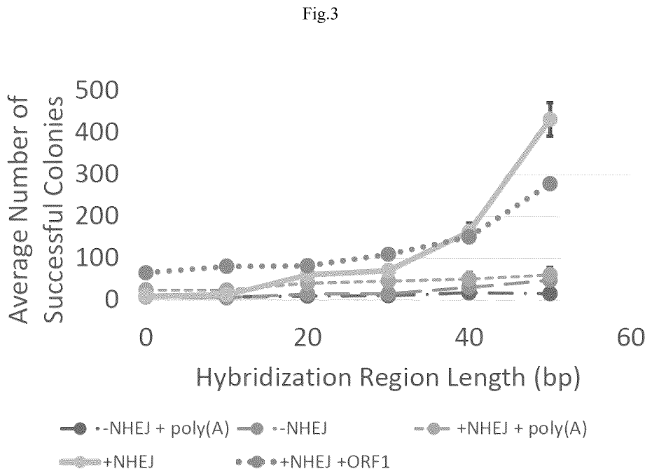

We designed and synthesized the GENEWRITE protein (see FIG. 1A, bottom) under control of a T7 promoter, which was cloned into the plasmid pUC57-kan (GENEWIZ Inc). We transformed this plasmid into strain BL21-AI, along with either empty plasmid pZA31, or pZA31 carrying ykoU and ykol B. subtilis NHEJ enzymes expressed from P_{LtetO1}. In strain BL21-AI, GENEWRITE expression is inducible by the addition of L-arabinose. We determined that fully induced expression of GENEWRITE without concomitant expression of NHEJ enzymes is lethal. We do not observe similar lethality with expression of either Cas9 or the ORF2pRT with the EN domain deleted alone (FIG. 4B). These results demonstrate the potential importance of NHEJ in function and survivability of GENEWRITE in E. coli.

### Homology of 40-50 bp Between Target and Payload RNA 3′ End Optimizes Reverse Transcription

As described below, we examined the ability of GENEWRITE to integrate RNA payloads delivered to E. coli. The strategy used is shown in FIG. 1B-1F. E. coli cells expressing GENEWRITE are transformed with a guide sgRNA to target Cas cleavage to the desired integration site, and a payload RNA carrying the coding sequence of the desired integration. The 3′ end of the payload RNA is designed to be homologous to the bottom DNA strand downstream from the cut to prime TPRT. Host enzymes complete second strand synthesis and payload RNA removal, and the insert is sealed into the site by NHEJ. For experiments described here, the ~1500 bp payload RNA consisted of an aadA spectinomycin resistance gene driven by a strong, constitutive lacIQ1 promoter and Shine-Dalgarno ribosomal binding site (RBS). Consequently, after performing the GENEWRITE protocol, cells were plated on spectinomycin to select for potentially successful integrants.

In order to maximize the number of potential targets, we chose as an initial integration target the high copy number plasmid pUC57-kan (~1000/cell). Design of the payload RNA 3′ hybridization region might be important for success. To determine the optimal length of the hybridization region in this experimental setting, we synthesized primers (Integrated DNA Technologies) to generate an array of six identical payload RNAs with 3′ hybridization length variable from 0-50 bp in 10 bp increments by in vitro transcription (T7 MEGAScript, ThermoFisher Scientific). We also generated a second array of payload RNAs, identical to the first, but also including the 30 bp poly(A) tract (SEQ ID NO: 41) found in AluYA5.

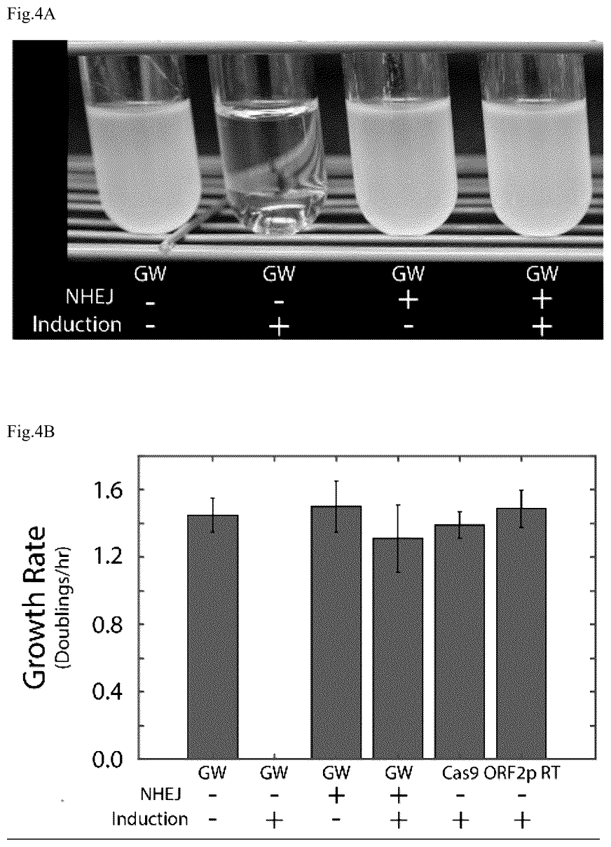

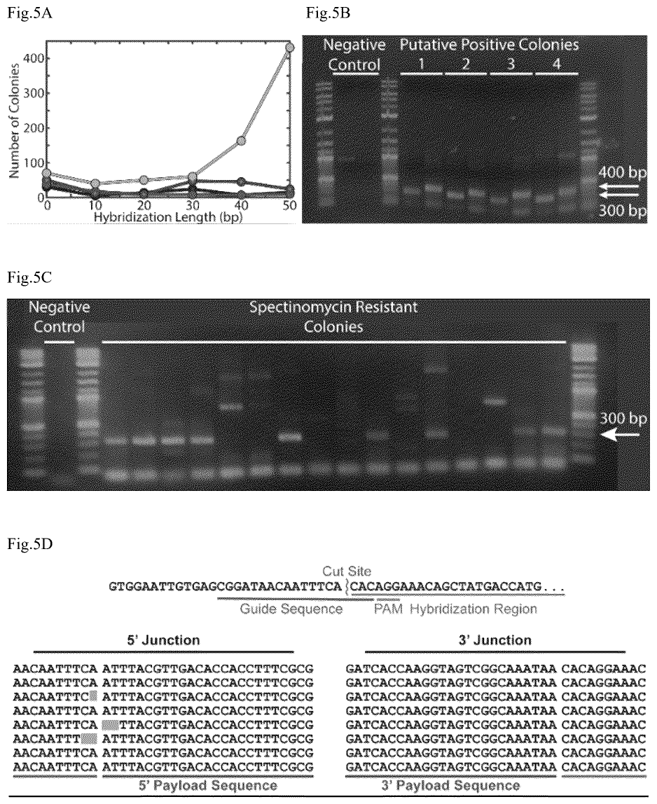

We transformed the sgRNA along with each payload RNA into E. coli weakly induced to express GENEWRITE, either with or without expression of B. subtilis NHEJ enzymes. Both cell density and RNA concentration were carefully controlled to ensure repeated experiments were performed identically. The results are shown in FIG. 5. For those payload RNAs containing a poly(A) tract, we observed very few spectinomycin resistant colonies, either with or without simultaneous co-expression of NHEJ (FIG. 5A, red). Conversely, without the poly(A) tract, we obtained hundreds of spectinomycin resistant colonies when complemented with simultaneous expression of NHEJ (FIG. 5A, light cyan). Controls delivering only sgRNA or only payload yielded very few (0-10) resistant colonies (FIG. 6, columns 2, 3). From these experiments, we conclude that optimal deployment of GENEWRITE may include a payload RNA with about 40-50 bp of homology to the target at the 3′ end, facilitated by NHEJ DNA repair.

GENEWRITE Specifically Integrates Payloads into Targeted Plasmid Locations with ~72% Success

Site-specific integration was verified by PCR using primers which amplified across the 5′ and 3′ integration junctions (FIG. 5B); 63/96 colonies screened yielded a positive signal for a success rate of ~72% (FIG. 5C). Sequencing of eight purified plasmids revealed some small deletions at the 5′ end of the insertion, perhaps due to NHEJ repair (FIG. 5D).

### The 5′ GAL4 Domain is Important for GENEWRITE Function

We included a GAL4 domain towards the N-terminal end of the GENEWRITE protein (see FIG. 1A, bottom); it is this version of the protein used to generate the above described results. We repeated the above procedure using a version of the GENEWRITE protein with the GAL4 domain removed, where we target the same integration site with a payload including 40 bp homology to the target. The results (FIG. 6, fourth column) illustrate that the GAL4 domain is necessary for GENEWRITE function in this experimental setting.

GENEWRITE with Cas12a/Cpf1

To test the generality of the GENEWRITE approach with respect to the employed endonuclease, we replaced Cas9 with Cas12a/Cpf1, a Cas enzyme which generates staggered cuts with 5′ overhang. We find that this alternative GENEWRITE also functions (FIG. 6, fifth column).

### Targeted Chromosomal Integration

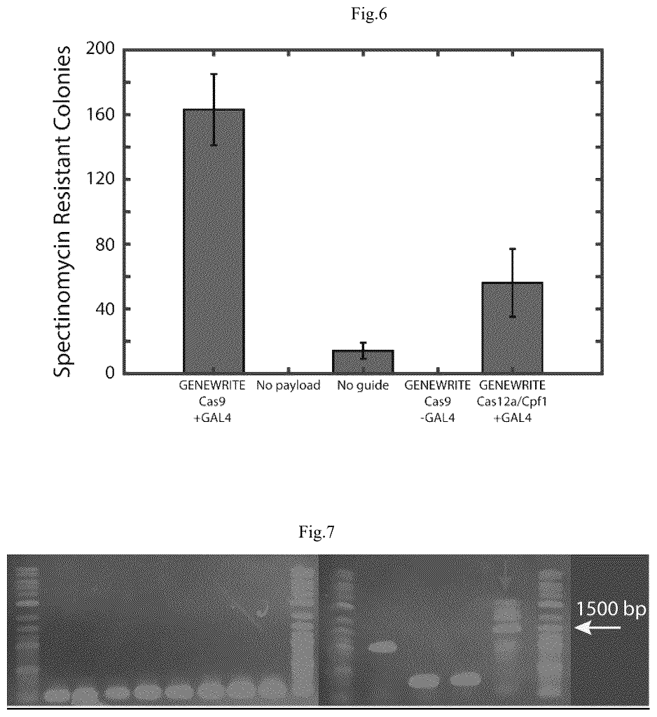

We next attempted to target insertion to single copy chromosomal loci using GENEWRITE-Cas9. A representative example, where we targeted the nth locus located near the terminus of replication is shown in FIG. 7. We generated an sgRNA to target this locus as well as the same ~1500 bp aadA payload described above with 40 bp homology to the target site. We obtained ~50 spectinomycin resistant colonies on average. PCR screening of 12 representative colonies is shown in FIG. 7, where PCR product from successful integration should be ~1500 bp, and where we identify one successful integrant (green arrow). This integrant was further verified by sequencing.

LINE-1 Integration Locations in E. coli.

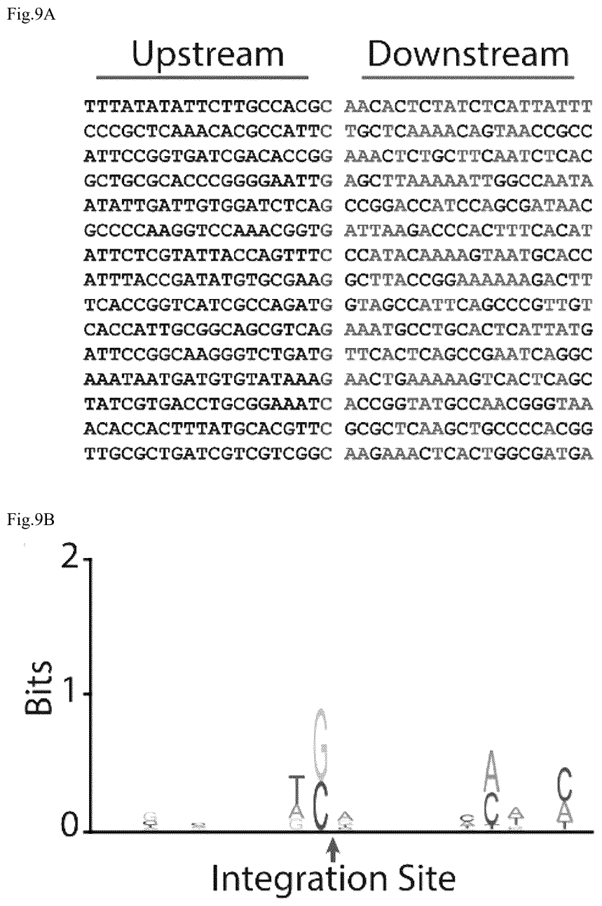

We have performed whole genome sequencing on E. coli that have been exposed to prolonged LINE-1 expression to identify integration locations. LINE-1 expression in these strains is driven by a T7 promoter, and the first three bases of the resulting transcript are TGA. 15 identified LINE-1 integration locations are shown in FIG. 9. While the regions downstream of the cut are T-A rich (55% TA), consistent with poly(A) tract hybridization initiating TPRT, the most prominent feature is that the first base upstream of the integration site is G/C in 100% of integration locations. Our attempts described above were performed without consideration of the insertion site upstream sequence.

### Optimization of the 5′ End of the Payload RNA

To optimize design of the 5′ end of the payload, an aadA spectinomycin resistance payload is used as described above, which is targeted towards the nth chromosomal locus (located near the terminus), atpI locus (located near the origin of replication), and ybbD locus (midway between origin and terminus on the right replichore), with 40 bp of 3′ homology to each of these targets. By synthesizing primers with the required sequence (IDT), we generate an array of payloads by PCR and in vitro transcription with 1-20 bp of homology on the 5′ end, in 5 bp increments, to the upstream side of the cut generated by Cas9 at each target site (i.e., the red sequence in FIG. 1C-F). These payloads, both with and without sgRNAs, are transformed into E. coli BL21-AI pUC57-kan-GENEWRITE and the success rate is determined by PCR screening and sequencing of identified spectinomycin resistant colonies, as above (see, FIGS. 5A-5D).

### Effects of ORF1p on Integration Efficiency

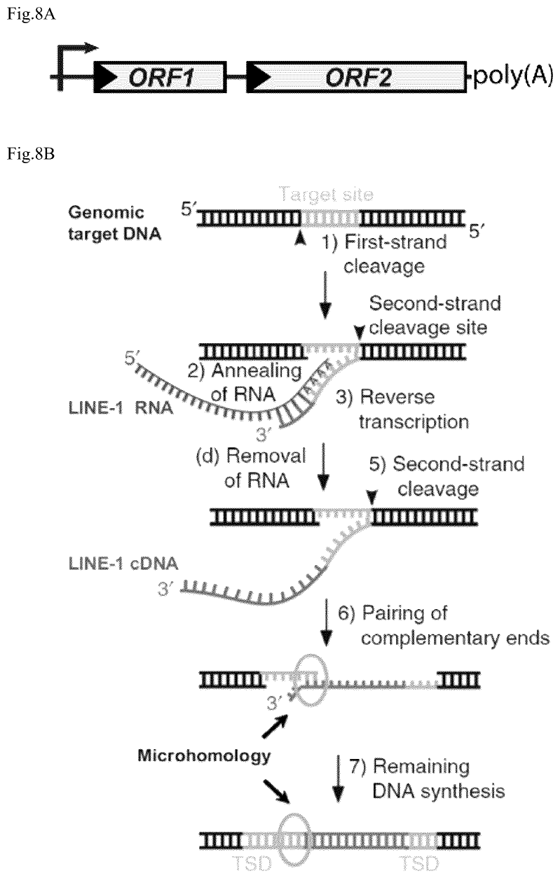

The GENEWRITE coding sequence on the plasmid pUC57-kan-GENEWRITE is modified to include the gene encoding ORF1 upstream of the GENEWRITE gene, mimicking the natural arrangement of ORF1 and ORF2 in LINE-1 (FIG. 8A). E. coli is transformed with either pUC57-kan-GENEWRITE or pUC57-GENEWRITE+ORF1, along with pZA31-NHEJ. Using an aadA payload and targeted towards the atpI, ybbD, and nth chromosomal loci, E. coli BL21-AI pUC57-kan-GENEWRITE is transformed, along with requisite sgRNAs, and the number of spectinomycin resistant colonies is quantified with correct payload integrations through colony PCR screening and sequencing to quantify the effect of ORFIp expression on integration efficiency (FIG. 5).

### RNP Assembly and Characterization.

By T7 in vitro transcription, pools of LINE-1, aadA payload, lacZ payload, and RNAs from pTKIP plasmid backbone are generated. These RNAs are mixed with either purified GENEWRITE or ORF2p protein in binding buffer (50 mM Tris-HCl pH 7.5, 150 mM NaCl, 5% glycerol, 0.1 mg/ml BSA, 2 mM DTT, 2 mM MgCl₂), and reactions are incubated at 25° C. for 30 min. sgRNA is added and again incubated at 25° C. for 30 min. Purified ORF1p protein is then added and again incubated at 25° C. for 30 min. These reactions are separated by SDS-PAGE, and protein amounts quantified by Western blot using rabbit anti-ORF1p antibody (Leibold et al., Proc Natl Acad Sci USA. 1990; 87 (18): 6990-4), rabbit anti-ORF2p antibody (Goodier et al., Human molecular genetics. 2004; 13 (10): 1041-8.), rabbit anti-GAL4 antibody [(Christian et al., Nucleic Acids Research. 2016; 44 (10): 4818-34); Santa Cruz Biotechnology], and f horseradish peroxidase (HRP) conjugated goat anti-rabbit secondary antibodies (Fisher Scientific). RNPs assembled using LINE-1 and GENEWRITE proteins are compared to determine their relative stoichiometry.

GENEWRITE RNPs assembled in vitro may be used for direct delivery to cells.

### Example 3

### Evaluation of GENEWRITE in Human Cells

What differentiates GENEWRITE from other existing genome editing technologies is the ability to insert large genetic payloads to specific sites in the genome. Such a strategy could prove beneficial in the repair or replacement of gene microdeletions where gene dosage and chromosomal context are important. Examples of human microdeletion disorders that could be addressed with GENEWRITE include, e.g., Li-Fraumeni syndrome (TP53 deletion) Angelman syndrome (UBE3A deletion), neurofibromatosis type I and II (NF-1 and Merlin deletion), Rubinstein-Taybi syndrome (CREBBP and/or EP 300 deletion) and many cancers associated with deletion of the tumor suppressor TP53.

As described below, the GENEWRITE system is delivered to human cell lines. The delivery mechanisms are first optimized and the payload design principles are verified by delivering GFP and puromycin resistance to Jurkat cells. Subsequently, the TP53 tumor suppressor gene in TP53 NULL human cancer cell lines is replaced using GENEWRITE. The experiments described below use a GENEWRITE protein, e.g., as described in Example 1.

### Delivery of GFP/Puromycin Resistance to Various Loci in Jurkat Cells

Delivery mechanisms are optimized and the payload design principles are verified by delivering GFP and puromycin resistance to Jurkat cells. Arrays of payload RNAs, e.g., as described in Example 2, as well as additional sets of payload RNAs including the 30 bp poly(A) tract (SEQ ID NO: 41) from AluYA5, are generated. In Jurkat cells, integration is targeted to CCR5 and CD4 genomic loci, for which optimal sgRNAs have been designed and which have been well characterized for their receptivity towards Cas9 mediated editing (Dang et al., Genome Biology. 2015; 16 (1): 280). As a payload, a ~2,200 bp RNA with a constitutive EF1α promoter expressing EGFP and puromycin resistance is used. The GENEWRITE genome editing system is delivered using delivery methods described in this application (e.g., electroporation, or transfection to deliver GENEWRITE component protein(s), RNA(s), vector(s) or pre-assembled RNPs). Additionally, Deterministic Mechanoporation (DMP) (Dixit et al., Nano letters. 2020; 20 (2): 860-7, which is incorporated by reference in its entirety for all purposes) may also be used for delivery. Puromycin is added 48 hours after transfection in order to select for successful integrants. Surviving cells are imaged to verify GFP expression, and genomes purified for PCR screening and sequencing to verify appropriate integration.

### Delivery of TP53 Coding Sequence to NCI-H1299 Cancer Cell Line.

Mutations in TP53 occur in almost every type of cancer at rates from 38%-50% (Olivier et al., Cold Spring Harbor perspectives in biology. 2010; 2 (1): a001008-a), and restoration of p53 expression stops tumor growth and can induce senescence or apoptosis. As described herein, GENEWRITE is used to insert the 1,179 bp coding sequence of the primary isoform of p53 into the native TP53 gene locus on chromosome 17p13.1 in cell line NCI-H1299, a non-small cell lung carcinoma cell line that is homozygous TP53 NULL (Lin et al., The Journal of biological chemistry. 1996; 271 (25): 14649-52; Giaccone et al., Cancer research. 1992; 52 (9 Suppl): 2732s-6s). In the payload the following are included: (1) a constitutive EFla promoter driving expression of a puromycin cassette for easy selection of successful integrants; (2) a TET-ON promoter for inducible expression upon addition of doxycycline (Das et al., Curr Gene Ther. 2016; 16 (3): 156-67); and (3) the 1,179 bp coding sequence of the primary isoform of p53. The full size of this payload is ~3,000 bp. GENEWRITE, sgRNA, and TP53 payload are delivered to NCI-H1299 cells using DMP or delivery methods described in this application (e.g., electroporation, or transfection to deliver GENEWRITE component protein(s), RNA(s), vector(s) or pre-assembled RNPs). Puromycin is added to the cells 48 hours post transfection, and surviving cells undergo PCR screening and sequencing to verify integration.

### Delivery of TP53 Gene Sequence to NCI-H1299 Cancer Cell Line.

The native gene includes 13 exons and generates multiple isoforms through alternative splicing, and an imbalance in the expression of different p53 isoforms leads to cancer, premature aging, neurodegenerative diseases, inflammation, embryo malformations, and defects in tissue regeneration. Consequently, to replace TP53 to rescue anomalous phenotypes requires replacement of the entire coding sequence to provide native expression levels of all possible spice variants. The entire gene sequence for TP53, including all introns and exons, is 19,149 bp long, and the promoter and regulatory regions reside within the noncoding exon 1 (Tano et al., Identification of Minimal p53 Promoter Region Regulated by MALATI in Human Lung Adenocarcinoma Cells. Frontiers in Genetics. 2018; 8 (208); Tuck et al., Molecular and cellular biology. 1989; 9 (5): 2163-72). sgRNAs and RNA payloads are designed to integrate the TP53 coding sequence into the native 17p13.1 locus of cell line NCI-H1299. GENEWRITE, sgRNA, and the TP53 payload are delivered to NCI-H1299 cells using DMP or delivery methods described in this application (e.g., electroporation, or transfection to deliver GENEWRITE component protein(s), RNA(s), vector(s) or pre-assembled RNPs). Puromycin is added to cells 48 hours post transfection, and surviving cells undergo PCR screening and sequencing to verify integration.

For larger RNA payloads, integration may also be performed iteratively though successive applications. Integrations are verified by PCR screening and sequencing at each step.

Experiments described above are also performed in other cell types, including, e.g., in Saos-2 (osteosarcoma), HL60 (acute promyelocytic leukemia), and KATO-III (carcinoma) TP53 NULL cell lines. In certain embodiments, a fluorescent reporter is included at the N or C terminal ends of the TP53 gene sequence, such that successful integration results in a more readily apparent phenotype beyond entrance into senescence.

All publications, accession numbers, patents, and patent documents are incorporated by reference herein, as though individually incorporated by reference. The invention has been described with reference to various specific and preferred embodiments and techniques. However, it should be understood that many variations and modifications may be made while remaining within the spirit and scope of the invention.

1-25. (canceled)

26. A genome editing system in a bacterial cell, which system comprises:
1) a guide RNA or a vector comprising a nucleic acid encoding the guide RNA;
2) a payload RNA or a vector comprising a nucleic acid encoding the payload RNA, wherein the payload RNA comprises a 3′ homology end, which can hybridize to a target chromosomal genome sequence of the bacterial cell; and
3) a recombinant polypeptide or a vector encoding the recombinant polypeptide, wherein the polypeptide comprises the following operably linked amino acid sequences, listed in order from N-terminus to C-terminus: a Cas nuclease amino acid sequence that has at least about 80% sequence identity to SEQ ID NO:1 or SEQ ID NO:2, and an ORF2p reverse transcriptase (RT) amino acid sequence that has at least about 80% sequence identity to SEQ ID NO:3,
wherein the recombinant polypeptide lacks a functional ORF2p endonuclease (EN) domain,
wherein the guide RNA, the payload RNA, and the recombinant polypeptide are comprised in the bacterial cell, and
wherein the payload sequence encoded by the payload RNA is integrated into the target chromosomal genome of the bacterial cell.

27. The genome editing system of claim 26, wherein the recombinant polypeptide does not comprise an ORF2p endonuclease (EN) amino acid sequence.

28. The genome editing system of claim 26, wherein the 3′ homology end of the payload RNA has a length of about 30-50 nt.

29. The genome editing system of claim 26, wherein the payload RNA further comprises a 5′ homology end, which can hybridize to the target chromosomal genome sequence of the bacterial cell.

30. The genome editing system of claim 29, wherein the 5′ homology end of the payload RNA has a length longer than 15 nt but shorter than 50 nt.

31. The genome editing system of claim 30, wherein the 5′ homology end of the payload RNA has a length of at least 20 nt.

32. The genome editing system of claim 29, wherein the payload RNA consists of the 5′ homology end, a genetic payload, and the 3′ homology end.

33. The genome editing system of claim 26, wherein the payload RNA has a length of at least 1000 nt.

34. The genome editing system of claim 26, wherein the recombinant polypeptide comprises the following operably linked amino acid sequences, listed in order from N-terminus to C-terminus: a GAL4 DNA binding domain (DBD) amino acid sequence, the Cas nuclease amino acid sequence, and the ORF2p reverse transcriptase (RT) amino acid sequence.

35. The genome editing system of claim 34, wherein the GAL4 DBD amino acid sequence has at least about 85% sequence identity to SEQ ID NO:4.

36. The genome editing system of claim 26, wherein the recombinant polypeptide comprises an ORF2p amino acid sequence having at least about 85% sequence identity to SEQ ID NO:14.

37. The genome editing system of claim 26, wherein the recombinant polypeptide comprises an amino acid sequence having at least about 85% sequence identity to SEQ ID NO: 7 or SEQ ID NO:8.

38. The genome editing system of claim 26, further comprising one or more nonhomologous end joining (NHEJ) proteins, or one or more vectors comprising nucleic acids encoding one or more NHEJ proteins.

39. The genome editing system of claim 38, wherein the one or more NHEJ proteins comprise two Bacillus subtilis NHEJ proteins, which are Ku and LigD.

40. The genome editing system of claim 38, further comprising an ORF1p protein that comprises an amino acid sequence having at least about 85% sequence identity to SEQ ID NO:9, or a vector comprising a nucleic acid encoding the ORFlp protein.

41. The genome editing system of claim 26, wherein the Cas nuclease amino acid sequence has at least about 90% sequence identity to SEQ ID NO:1.

42. The genome editing system of claim 26, wherein the Cas nuclease amino acid sequence has at least about 90% sequence identity to SEQ ID NO:2.

43. The genome editing system of claim 26, wherein the recombinant polypeptide comprises one single Cas nuclease domain.

44. A method of editing a target chromosomal genome sequence in a bacterial cell, which method comprises contacting the bacterial cell with the genome editing system according to claim 26, wherein the components of the system are delivered to the bacterial cell under conditions suitable for the components of the system to enter the bacterial cell and edit the target chromosomal genome sequence.

45. The method of claim 44, wherein
a) the guide RNA, the target chromosomal genome sequence, and the recombinant polypeptide form a first protein-RNA-DNA complex in the bacterial cell, wherein the target chromosomal genome sequence is cut by the Cas nuclease of the recombinant polypeptide; and
b) the payload RNA, the target chromosomal genome sequence, and the recombinant polypeptide form a second protein-RNA-DNA complex in the bacterial cell, wherein the payload RNA 3′ homology end hybridizes with the target chromosomal genome sequence.
